(19) United States
(12) Patent Application Publication
(10) Pub. No.: US 2025/0205286 A1
(43) Pub. Date: Jun. 26, 2025

(54) COMPOSITIONS, REAGENTS, AND METHODS FOR TREATING PITT-HOPKINS SYNDROME

(71) Applicant: Vitro Biopharma, Inc., Golden, CO (US)

(72) Inventors: James R. MUSICK, Golden, CO (US); Tiana States, Golden, CO (US); Caroline MOSESSIAN, Golden, CO (US)

(21) Appl. No.: 18/849,678

(22) PCT Filed: Mar. 27, 2023
(86) PCT No.: PCT/US2023/016462

§ 371 Date: Sep. 23, 2024

Related U.S. Application Data
(60) Provisional application No. 63/269,966, filed on Mar. 25, 2022.

(51) Int. Cl. A61K 35/28; A61K 9/00; A61K 31/075; A61K 45/06; A61P 25/00; C12N 5/0775

(52) U.S. Cl. CPC A61K 35/28; A61K 9/0019; A61K 31/075; A61K 45/06; A61P 25/00; C12N 5/0665

(57) ABSTRACT

Provided herein are gene and cell therapy-based compositions and methods of treating Pitt-Hopkins Syndrome (PTHS). The methods of treating PTHS utilize compositions that contain an effective amount of umbilical cord-derived mesenchymal stem cells (UC-MSCs), isolated exosomes derived from the UC-MSCs, cell culture medium containing the UC-MSC's secretome (e.g., UC-MSC secretome-conditioned cell culture medium), and cell culture medium containing the UC-SLMSC's secretome but devoid of exosomes (e.g., exosome-depleted UC-MSC-conditioned cell culture medium). The compositions described herein additionally contain Wnt pathway activators to increase transcription factor 4 (TCF4 expression) in MSCS.

### BACKGROUND

Rare 18q chromosomal deletions and loss of function point mutations of transcription factor 4 (TCF4) are the molecular cause of Pitt-Hopkins Syndrome (PTHS), a rare genetic form of autism. Knock out of TCF4 affects the differentiation of specific neuronal populations in the mouse hindbrain (Flora A, et al., Proc Natl Acad Sci USA 2007; 104:15382-7). Experimental knockdown of TCF4 expression in human neuroblastoma-derived cells (SH-SY5Y) has been found to alter the expression of genes involved in transforming growth factor (TGF)-beta signaling, epithelial to mesenchymal transition, and apoptosis (Forrest M P, et al., PLoS One 2013; 8:e73169). Stable knockdown of TCF4 in neural progenitor cells from the human fetal midbrain has been reported to result in gene expression changes more characteristic of differentiating than proliferating cells, suggesting effects on the timing of neural differentiation (Chen E S, et al., Am J Hum Genet 95:490-508, 2014). The effects of reduced endogenous TCF4 expression by RNA interference were determined in a neural progenitor cell line derived from the developing human cerebral cortex. Genome-wide gene expression was assessed by microarray and pathway analysis of differentially expressed genes. Genes that were differentially expressed following TCF4 knockdown were highly enriched for involvement in the cell cycle, thus supporting a PTHS etiology involving reduction of proliferation of neural progenitor cells in the developing brain (Hill, M J, et al., J Psychiatry Neurosci. 42: 181-188, 2016).

PTHS can cause severe developmental delays and behavioral difficulties in children. Despite the growing knowledge of the genetic factors and molecular mechanisms underlying PTHS, there remains a need for new therapeutic methods for treating PTHS.

There is a growing body of evidence showing that mesenchymal stem cells (MSCs) regenerate neural tissues and restore neurogenesis. Recent reports have demonstrated that treatment with MSCs derived from human umbilical cord blood (HUCB) reduced post-stroke brain damage, inflammation, and apoptosis, improved the survival rate, and facilitated the neurological recovery of stroke-induced rats, rabbits, and canines (Chung, D J, et al., Journal of Neuroscience Research, 87(16), 3554-3567, 2009; Lim, J Y, et al., Stem Cell Research & Therapy, 2: 38, 2011; Kim, E S, et al., Pediatric Research, 72(3), 277-284, 2012; Zhu, Y., Acta Pharmaco logica Sinica, 35: 585-591, 2014; Chelluboina, B, et al., Neurochemical Research, 39(8), 1511-1521, 2014). In addition, the administration of these cells in animal models of ischemic stroke prevented the post-ischemic induction of matrix metalloproteinases, downregulated the DNA damage-inducing genes, and upregulated the DNA repair genes (Chelluboina, B, et al., Journal of Stem Cell Research & Therapeutics, 1: 281-288, 2016; Chelluboina, B, et al., Cellular Physiology and Biochemistry, 44(4), 1360-1369, 2017). This has been extended to various clinical studies of MSC therapy for stroke. The proliferative effects of MSCs do not appear to be specific to MSCs and may be reproduced with MSC-derived exosomes with varying effects depending on the cell culture conditions used to generate the exosomes, e.g., hypoxic conditions yielding more potent exosomes (Nalamolu et al. Cell Physiol Biochem 52:1280-1291, 2019; Nalamolu, K R, et al, Neuromol Med 21: 529-539, 2019).

### SUMMARY OF THE DISCLOSURE

The present disclosure provides methods of treating a subject with Pitt-Hopkins Syndrome (PTHS) and compositions for use thereof.

A first aspect of the disclosure features a method of treating a subject with Pitt-Hopkins Syndrome (PTHS) including administering to the subject an effective amount of a composition that includes: (a) a plurality of umbilical cord-derived human mesenchymal stem cells (UC-MSCs), wherein the UC-MSCs express transcription factor 4 (TCF4); (b) a plurality of isolated exosomes about 80-200 nanometers (nm) in diameter, wherein the isolated exosomes are derived from the UC-MSCs; (c) UC-MSC secretome-conditioned cell culture medium; and/or (d) exosome-depleted UC-MSC-conditioned cell culture medium.

In some aspects, the composition is administered intravenously, intra-articularly, intramuscularly, intranasally, or intrathecally. In some aspects, the composition is administered intravenously. In some aspects, the composition is administered intravenously by infusion.

In some aspects, the composition is administered in a volume of about 0.5 milliliters (mL) to about 15 mL (e.g., 0.5 mL, 0.6 mL, 0.7 mL, 0.8 mL, 0.9 mL, 1 mL, 3 mL, 5 mL, 7 mL, 10 mL, 11 mL, 12 mL, 13 mL, 14 mL, or 15 mL). In some aspects, the composition is administered in a volume of about 1 mL to about 10 mL (e.g., 1 mL, 2 mL, 3 mL, 4 mL, 5 mL, 6 mL, 7 mL, 8 mL, 9 mL, or 10 mL). In some aspects, the composition is administered in a volume of about 1 mL, about 2 mL, about 3 mL, about 4 mL, about 5 mL, or about 6 mL.

In some aspects, the composition is administered in a bolus or is administered over a period of about 1 minute to about 1 hour (e.g., 1 minute, 10 minutes, 20 minutes, 30 minutes, 40 minutes, 50 minutes, or 1 hour). In some aspects, the composition is administered over a period of about 1 minute to about 30 minutes (e.g., 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 21 minutes, 22 minutes, 23 minutes, 24 minutes, 25 minutes, 26 minutes, 27 minutes, 28 minutes, 29 minutes, or 30 minutes). In some aspects, the composition is administered over a period of about 1 minute to about 10 minutes (e.g., 1 minute, 1.5 minutes, 2 minutes, 2.5 minutes, 3 minutes, 3.5 minutes, 4 minutes, 4.5 minutes, 5 minutes, 5.5 minutes, 6 minutes, 6.5 minutes, 7 minutes, 7.5 minutes, 8 minutes, 8.5 minutes, 9 minutes, 9.5 minutes, or 10 minutes).

In some aspects, the composition is administered at a frequency of once every one, two, three, four, five, or six months. In some aspects, the composition is administered at a frequency of once every month. In some aspects, the composition is administered at a frequency of once every two months. In some aspects, the composition is administered at a frequency of once every three months. In some aspects, the composition is administered at a frequency of once every four months. In some aspects, the composition is administered at a frequency of once every five months. In some aspects, the composition is administered at a frequency of once every six months. In some aspects, the composition is administered at a frequency of once every seven months. In some aspects, the composition is administered at a frequency of once every eight months. In some aspects, the composition is administered at a frequency of once every nine months. In some aspects, the composition is administered at a frequency of once every ten months. In some aspects, the composition is administered at a frequency of once every eleven months. In some aspects, the composition is administered at a frequency of once every twelve months.

In some aspects, the UC-MSCs are modified to increase TCF4 mRNA and/or protein expression levels relative to unmodified UC-MSCs. In some aspects, the TCF4 mRNA and/or protein expression levels are increased 5%, 10%, 15%, 20%, 25%, 30%, 35%, 40%, 45%, 50%, 55%, 60%, 65%, 70%, 75%, 80%, 85%, 90%, 95%, 100%, or more (e.g., 150%, 200%, 250%, 300%, 250%, or 400%), relative to an unmodified UC-MSC.

In some aspects, the modified UC-MSCs include a nucleic acid vector, plasmid, circular RNA, or mRNA molecule including a nucleotide sequence encoding a TCF4 protein and/or a frizzled-signaling agonist. In some aspects, the frizzled-signaling agonist is Wnt-3a protein.

In some aspects, the method further includes, prior to administering the composition, contacting the UC-MSCs with a Wnt pathway activator. In some aspects, the Wnt pathway activator is selected from the group including a histone deacetylase 1 (HDAC1) inhibitor, a Wnt-signaling agonist, a frizzled-signaling agonist, and a glycogen synthase kinase-3p (GSK-3B13P) inhibitor.

In some aspects, the HDAC1 inhibitor is selected from the group including vorinostat, romidepsin, belinostat, panobinostat, valproic acid, entinostat, curcumin, curcumin, quercetin, and RG2833. In some aspects, the Wnt-signaling agonist is L-Quebrachital. In some aspects, the frizzled-signaling agonist is a Wnt agonist-1 protein or a Wnt-3a protein. In some aspects, the GSK-3B13P inhibitor is selected from the groups including indirubin-3′-oxime, laduviglusib (CHIR-99821), and KY19382.

In some aspects, the contacting is for about 1 day to about 6 weeks (e.g., 1 day, 2 days, 3 days, 4 days, 5 days, 6 days, 1 week, 2 weeks, 3 weeks, 4 weeks, 5 weeks, or 6 weeks). In some aspects, the contacting is for about 1-3 weeks (e.g., 1 week, 2 weeks, or 3 weeks). In some aspects, the contacting is for about 2 weeks (e.g., 13 days, 14 days, or 15 days).

In some aspects, the HDAC1 inhibitor is at a concentration of about 1 nanomolar (nM) to about 10 micromolar (μM) (e.g., 1 nM, 50 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, or 10 PM). In some aspects, the HDAC1 inhibitor is at a concentration of about 100 nM to about 1 μM (e.g., 100 nM, 150 nM, 200 nM, 250 nM, 300 nM, 350 nM, 400 nM, 450 nM, 500 nM, 550 nM, 600 nM, 650 nM, 700 nM, 750 nM, 800 nM, 850 nM, 900 nM, 950 nM, or 1 μM). In some aspects, the HDAC1 inhibitor is at a concentration of about 400 nM to about 600 nM (e.g., 400 nM, 425 nM, 450 nM, 475 nM, 500 nM, 525 nM, 550 nM, 575 nM, or 600 nM). In some aspects, the HDAC1 inhibitor is at a concentration of about 500 nM (e.g., 450 nM, 460 nM, 470 nM, 480 nM, 490 nM, 500 nM, 510 nM, 520 nM, 530 nM, 540 nM, or 550 nM).

In some aspects, the Wnt-3a protein is at a concentration of about 5 ng/mL to about 20 ng/mL (e.g., 5 ng/mL, 6 ng/mL, 7 ng/mL, 8 ng/mL, 9 ng/mL, 10 ng/mL, 11 ng/mL, 12 ng/mL, 13 ng/mL, 14 ng/mL, 15 mg/mL, 16 ng/mL, 17 ng/mL, 18 ng/mL, 19 ng/mL, or 20 ng/mL).

In some aspects, the subject is administered a Wnt pathway activator prior to administration of the composition. In some aspects, the Wnt pathway activator is selected from the group including a HDAC1 inhibitor, a Wnt-signaling agonist, a frizzled-signaling agonist, and a GSK-3B13P inhibitor.

In some aspects, the HDAC1 inhibitor is selected from the group including vorinostat, romidepsin, belinostat, panobinostat, valproic acid, entinostat, curcumin, curcumin, quercetin, and RG2833. In some aspects, the Wnt-signaling agonist is L-Quebrachital. In some aspects, the frizzled-signaling agonist is a Wnt agonist-1 protein or a Wnt-3a protein. In some aspects, the GSK-3B13P inhibitor is selected from the groups including indirubin-3′-oxime, laduviglusib (CHIR-99821), and KY19382.

In some aspects, the HDAC1 inhibitor is administered to the subject in an amount sufficient to achieve a serum concentration of the HDAC1 inhibitor of about 0.1 nM to about 1,000 nM (e.g., about 0.1 nM to 10 nM, about 1 nM to about 100 nM, about 10 nM to about 1000 nM, about 200 nM to about 500 nM, about 350 nM to about 700 nM, or about 500 nM to about 1000 nM, e.g., 01 nM, 0.5 nM, 1 nM, 5 nM, 10 nM, 20 nM, 30 nM, 40 nM, 50 nM, 60 nM, 70 nM, 80 nM, 90 nM, 100 nM, 125 nM, 150 nM, 175 nM, 200 nM, 225 nM, 250 nM, 275 nM, 300 nM, 325 nM, 350 nM, 375 nM, 400 nM, 425 nM, 450 nM, 475 nM, 500 nM, 525 nM, 550 nM, 575 nM, 600 nM, 625 nM, 650 nM, 675 nM, 700 nM, 725 nM, 750 nM, 775 nM, 800 nM, 825 nM, 850 nM, 875 nM, 900 nM, 925 nM, 950 nM, 975 nM, or 1000 nM). In some aspects, the HDAC1 inhibitor is administered to the subject in an amount sufficient to achieve a serum concentration of the HDAC1 inhibitor of about 500 nM (e.g., 450 nM, 460 nM, 470 nM, 480 nM, 490 nM, 500 nM, 510 nM, 520 nM, 530 nM, 540 nM, or550 nM).

In some aspects, the Wnt-3a protein is administered to the subject in an amount sufficient to achieve a serum concentration of the Wnt-3a protein of about 5 ng/mL to about 20 ng/mL (e.g., 5 ng/mL, 6 ng/mL, 7 ng/mL, 8 ng/mL, 9 ng/mL, 10 ng/mL, 11 ng/mL, 12 ng/mL, 13 ng/mL, 14 ng/mL, 15 mg/mL, 16 ng/mL, 17 ng/mL, 18 ng/mL, 19 ng/mL, or 20 ng/mL).

In some aspects, the method further includes administering to the subject a Wnt pathway activator concurrently with or following administration of the composition. In some aspects, the Wnt pathway activator is selected from the group including a HDAC1 inhibitor, a Wnt-signaling agonist, a frizzled-signaling agonist, and a GSK-3B13P inhibitor.

In some aspects, the HDAC1 inhibitor is selected from the group including vorinostat, romidepsin, belinostat, panobinostat, valproic acid, entinostat, curcumin, curcumin, quercetin, and RG2833. In some aspects, the Wnt-signaling agonist is L-Quebrachital. In some aspects, the frizzled-signaling agonist is a Wnt agonist-1 protein or a Wnt-3a protein. In some aspects, the GSK-3B13P inhibitor is selected from the groups including indirubin-3′-oxime, laduviglusib (CHIR-99821), and KY19382.

In some aspects, the subject is administered vorinostat in an amount of about 400 mg and/or in an amount sufficient to achieve geometric mean values of a maximum plasma concentration (Cₘₐₓ) and an area under the plasma concentration versus time curve (AUC₀₋ᵢₙₜ) of about 1.2±0.53 μM and about 6.0±2.0 μM*hr, respectively. In some aspects, the subject is administered romidepsin in an amount of about 14 mg/m² IV over a 4-hour period, such as on days 1, 8, and 15 of a 28-day cycle and/or in an amount sufficient to achieve geometric mean values of a maximum plasma concentration (Cₘₐₓ) and an area under the plasma concentration versus time curve (AUC₀₋ᵢₙₜ of about 377 ng/mL and about 1549 ng*hr/mL, respectively. In some aspects, the subject is administered belinostat in an amount of about 1,000 mg/m² over a 30 minute period, such as on days 1-5 of a 21-day cycle. In some aspects, the subject is administered panobinostat in an amount of about 20 mg every other day, such as on days 1, 3, 5, 8, 10, and 12 of a 21-day cycle. In some aspects, the subject is administered valproic acid in an amount of about 10 to 60 mg/kg/day. In some aspects, the subject is administered entinostat in an amount of about 2 mg/m² to about 12 mg/m² per day. In some aspects, the subject is administered curcumin in an amount of about 1 g to about 8 g per day. In some aspects, the subject is administered quercetin in an amount of about 250 mg to about 1000 mg per day. In some aspects, the subject is administered RG2833 in an amount of about 30 mg to about 240 mg per day.

In some aspects, the subject is an infant, child, or adolescent. In some aspects, the infant is less than one year of age. In some aspects, the child is between one year and 10 years of age. In some aspects, the adolescent is over 10 years and under 19 years of age.

In some aspects, the infant is administered the composition by intravenous infusion at about 1×10⁶ to 2.5×10⁶ UC-MSCs per kilogram (kg) of body about 3 to 4 times per year depending on need.

In some aspects, the child is administered the composition by intravenous infusion at about 1×10⁶ to 2.5×10⁶ UC-MSCs per kilogram (kg) of body about 3 to 4 times per year depending on need. In some aspects, the adolescent is administered the composition by intravenous infusion at about 1×10⁶ to 2.5×10⁶ UC-MSCs per kilogram (kg) of body about 3 to 4 times per year depending on need.

In some aspects, the subject exhibits an impairment in motor function, communication, sleep, gastrointestinal health, breathing, cognition, and/or adaptive behavior. In some aspects, the motor function impairment is determined by one or more of a Vineland Motor Subscale-3 caregiver interview, a Bayley Scales of Infant Development (BSID-4) questionnaire, a video capture of gait in coronal and sagittal plane, a Functional Independence Measure for Children (WeeFIM), and an Observer-Reported Communication Ability Measure (ORCA). In some aspects, the communication impairment is determined by WeeFIM and/or ORCA. In some aspects, the sleep impairment is determined by a sleep diary questionnaire. In some aspects, the gastrointestinal impairment is determined by a gastrointestinal health questionnaire. In some aspects, the breathing impairment is determined by spirometry. In some aspects, the cognition impairment is determined by a BSID-4 questionnaire. In some aspects, the adaptive behavior impairment is determined by one or more of a Q-global Vineland assessment, a Vineland behavioral scalers questionnaire, or an Aberrant Behavior Checklist-2.

In some aspects, the subject has a reduced TCF4 expression level in excitatory neurons, inhibitory neurons, astrocytes, oligodendrocytes and/or lymphocytes relative to a healthy subject. In some aspects, the subject has a monoallelic mutation or deletion in TCF4 that reduces TCF4 expression, relative to a healthy subject without the mutation or deletion in TCF4.

In some aspects, the composition increases TCF4 mRNA and/or protein expression in the brain of the subject relative to the TCF4 expression level prior to administering of the composition.

In some aspects, the pharmaceutical composition further includes: (a) about 5×10⁵ to 5×10⁶ (e.g., 5×10⁵ to 1×10⁶, 5×10⁵ to 1.5×10⁶, 1×10⁶ to 2.5×10⁶, 1.5×10⁶ to 3×10⁶, 2×10⁶ to 5×10⁶, or 4×10⁶ to 5×10⁶, e.g., 5×10⁵, 6×10⁵, 7×10⁵, 8×10⁵, 9×10⁵, 1×10⁶, 1.5×10⁶, 2×10⁶, 2.5×10⁶, 3×10⁶, 3.5×10⁶, 4×10⁶, 4.5×10⁶, or 5×10⁶) of said UC-MSCs; (b) a concentration of about 5×10⁹ to 5×10¹⁰ (e.g., 5×10⁹ to 1.5×10¹⁰, 1×10¹⁰ to 3×10¹⁰, 2×10¹⁰ to 4×10¹⁰, 3×10¹⁰ to 5×10¹⁰, 1×10¹⁰ to 5×10¹⁰, or 2.5×10¹⁰ to 5×10¹⁰, e.g., 5×10⁹, 6×10⁹, 7×10⁹, 8×10⁹, 9×10⁹, 1×10¹⁰, 1.5×10¹⁰, 2×10¹⁰, 2.5×10¹⁰, 3×10¹⁰, 3.5×10¹⁰, 4×10¹⁰, 4.5×10¹⁰, or 5×10¹⁰) said exosomes per mL, said exosomes expressing cluster of differentiation (CD)-9, CD63, CD81, CD29, CD44 and/or CD144; (c) about 100 pg/mL to about 5000 pg/mL (e.g., 500 pg/mL to 1000 pg/mL, 800 pg/mL to 1600 pg/mL, or 1400 pg/mL to 2000 pg/mL, e.g., 500 pg/mL, 550 pg/mL, 600 pg/mL, 650 pg/mL, 700 pg/mL, 750 pg/mL, 800 pg/mL, 850 pg/mL, 900 pg/mL, 950 pg/mL, 1000 pg/mL, 1050 pg/mL, 1100 pg/mL, 1150 pg/mL, 1200 pg/mL, 1250 pg/mL, 1300 pg/mL, 1350 pg/mL, 1400 pg/mL, 1450 pg/mL, 1500 pg/mL, 1550 pg/mL, 1600 pg/mL, 1650 pg/mL, 1700 pg/mL, 1750 pg/mL, 1800 pg/mL, 1850 pg/mL, 1900 pg/mL, 1950 pg/mL, 2000 pg/mL, 3000 pg/mL, 4000 pg/mL, or 5000 pg/mL) of granulocyte-macrophage colony-stimulating factor (GM-CSF), macrophage inflammatory protein (MIP)-3 alpha (MIP-3a), IL-6, and IL-8 and about 10 pg/mL to about 1000 pg/mL (e.g., 10 pg/mL to 500 pg/mL, 250 pg/mL to 750 pg/mL, or 500 pg/mL to 1000 pg/mL, e.g., 10 pg/mL, 20 pg/mL, 30 pg/mL, 40 pg/mL, 50 pg/mL, 60 pg/mL, 70 pg/mL, 80 pg/mL, 90 pg/mL, 100 pg/mL, 150 pg/mL, 200 pg/mL, 250 pg/mL, 300 pg/mL, 350 pg/mL, 400 pg/mL, 450 pg/mL, 500 pg/mL, 600 pg/mL, 700 pg/mL, 800 pg/mL, 900 pg/mL, or 1000 pg/mL) of fractalkine and MIP-1; and/or (d) a pharmaceutically acceptable carrier, excipient, or diluent.

In some aspects, the composition does not contain DMSO. In some aspects, the composition includes further includes a cryopreservation medium, a basal medium, and/or a saline solution. In some aspects, the cryopreservation medium is PRIME-XV® MSC FreeziS DMSO-Free medium. In some aspects, the basal medium is MCDB-131.

In some aspects, the composition includes about 1×10⁶ to 2.5×10⁶ (e.g., 1.5×10⁶, 1.6×10⁶, 1.7×10⁶, 1.8×10⁶, 1.9×10⁶, 2×10⁶, 2.1×10⁶, 2.2×10⁶, 2.3×10⁶, 2.4×10⁶, or 2.5×10⁶) said UC-MSCs. In some aspects, the composition includes about 1×10⁶ said UC-MSCs.

In some aspects, the composition includes a concentration of about 1×10¹⁰ to 5×10¹⁰ (e.g., 1×10¹⁰, 1.5×10¹⁰, 2×10¹⁰, 2.5×10¹⁰, 3×10¹⁰, 3.5×10¹⁰, 4×10¹⁰, 4.5×10¹⁰, or 5×10¹⁰) said isolated exosomes per mL.

In some aspects, the exosomes express CD9, CD63, and CD81. In some aspects, the exosomes further express CD44, CD29, and CD412. In some aspects, the exosomes do not express CD45, CD11 b, CD14, CD19, CD34, CD79a, CD126, and human leukocyte antigen-DR isotype (HLD-DR).

In some aspects, the composition includes about 100 pg/mL to about 1000 pg/mL (e.g., 100 pg/mL to 500 pg/mL, 250 pg/mL to 750 pg/mL, or 500 pg/mL to 1000 pg/mL, e.g., 100 pg/mL, 150 pg/mL, 200 pg/mL, 250 pg/mL, 300 pg/mL, 350 pg/mL, 400 pg/mL, 5450 pg/mL, 500 pg/mL, 550 pg/mL, 600 pg/mL, 650 pg/mL, 700 pg/mL, 750 pg/mL, 800 pg/mL, 850 pg/mL, 900 pg/mL, 950 pg/mL, or 1000 pg/mL) of GM-CSF, MIP-3a, IL-6, and IL-8 and about 10 pg/mL to about 100 pg/mL (e.g., 10 pg/mL to 50 pg/mL, 25 pg/mL to 75 pg/mL, or 50 pg/mL to 100 pg/mL, e.g., 10 pg/mL, 20 pg/mL, 30 pg/mL, 40 pg/mL, 50 pg/mL, 60 pg/mL, 70 pg/mL, 80 pg/mL, 90 pg/mL, or 100 pg/mL) of fractalkine and MIP-1. In some aspects, the composition includes about 1000 pg/mL of GM-CSF, MIP-3a, IL-6, and IL-8 and about 100 pg/mL of fractalkine and MIP-1.

A second aspect of the disclosure features a pharmaceutical composition including: (a) a plurality of UC-MSCs, wherein the UC-MSCs express TCF4; (b) a plurality of isolated exosomes of about 80-200 nm in diameter, wherein the isolated exosomes are derived from the UC-MSCs and express CD9, CD63, CD81, CD44, CD29 and CD142; (c) UC-MSC secretome-conditioned cell culture medium; and/or (d) exosome-depleted UC-MSC-conditioned cell culture medium. In some aspects, the composition further includes one or more Wnt pathway activators.

In some aspects, the Wnt pathway activator is selected from the group including a HDAC1 inhibitor, a Wnt-signaling agonist, a frizzled-signaling agonist, and a GSK-3B13P inhibitor.

In some aspects, the HDAC1 inhibitor is selected from the group including vorinostat, romidepsin, belinostat, panobinostat, valproic acid, entinostat, curcumin, curcumin, quercetin, and RG2833. In some aspects, the Wnt-signaling agonist is L-Quebrachital. In some aspects, the frizzled-signaling agonist is a Wnt agonist-1 protein or a Wnt-3a protein. In some aspects, the GSK-3B13P inhibitor is selected from the groups including indirubin-3′-oxime, laduviglusib (CHIR-99821), and KY19382.

In some aspects, the UC-MSCs are modified to increase expression of TCF4 mRNA and/or protein expression levels relative to unmodified UC-MSCs. In some aspects, the modified UC-MSCs include a nucleic acid vector, plasmid, circular RNA, or mRNA molecule including a nucleotide sequence encoding a TCF4 protein and/or a frizzled-signaling agonist.

In some aspects, the pharmaceutical composition further includes: (a) about 5×10⁵ to 5×10⁶ (e.g., 5×10⁵ to 1×10⁶, 5×10⁵ to 1.5×10⁶, 1×10⁶ to 2.5×10⁶, 1.5×10⁶ to 3×10⁶, 2×10⁶ to 5×10⁶, or 4×10⁶ to 5×10⁶, e.g., 5×10⁵, 6×10⁵, 7×10⁵, 8×10⁵, 9×10⁵, 1×10⁶, 1.5×10⁶, 2×10⁶, 2.5×10⁶, 3×10⁶, 3.5×10⁶, 4×10⁶, 4.5×10⁶, or 5×10⁶) of said UC-MSCs; (b) a concentration of about 5×10⁹ to 5×10¹⁰ (e.g., 5×10⁹ to 1.5×10¹⁰, 1×10¹⁰ to 3×10¹⁰, 2×10¹⁰ to 4×10¹⁰, 3×10¹⁰ to 5×10¹⁰, 1×10¹⁰ to 5×10¹⁰, or 2.5×10¹⁰ to 5×10¹⁰, e.g., 5×10⁹, 6×10⁹, 7×10⁹, 8×10⁹, 9×10⁹, 1×10¹⁰, 1.5×10¹⁰, 2×10¹⁰, 2.5×10¹⁰, 3×10¹⁰, 3.5×10¹⁰, 4×10¹⁰, 4.5×10¹⁰, or 5×10¹⁰) said exosomes per mL; (c) about 100 pg/mL to about 5000 pg/mL (e.g., 500 pg/mL to 1000 pg/mL, 800 pg/mL to 1600 pg/mL, or 1400 pg/mL to 2000 pg/mL, e.g., 500 pg/mL, 550 pg/mL, 600 pg/mL, 650 pg/mL, 700 pg/mL, 750 pg/mL, 800 pg/mL, 850 pg/mL, 900 pg/mL, 950 pg/mL, 1000 pg/mL, 1050 pg/mL, 1100 pg/mL, 1150 pg/mL, 1200 pg/mL, 1250 pg/mL, 1300 pg/mL, 1350 pg/mL, 1400 pg/mL, 1450 pg/mL, 1500 pg/mL, 1550 pg/mL, 1600 pg/mL, 1650 pg/mL, 1700 pg/mL, 1750 pg/mL, 1800 pg/mL, 1850 pg/mL, 1900 pg/mL, 1950 pg/mL, 2000 pg/mL, 3000 pg/mL, 4000 pg/mL, or 5000 pg/mL) of GM-CSF, MIP-3a, IL-6, and IL-8 and about 10 pg/mL to about 1000 pg/mL (e.g., 10 pg/mL to 500 pg/mL, 250 pg/mL to 750 pg/mL, or 500 pg/mL to 1000 pg/mL, e.g., 10 pg/mL, 20 pg/mL, 30 pg/mL, 40 pg/mL, 50 pg/mL, 60 pg/mL, 70 pg/mL, 80 pg/mL, 90 pg/mL, 100 pg/mL, 150 pg/mL, 200 pg/mL, 250 pg/mL, 300 pg/mL, 350 pg/mL, 400 pg/mL, 450 pg/mL, 500 pg/mL, 600 pg/mL, 700 pg/mL, 800 pg/mL, 900 pg/mL, or 1000 pg/mL) of fractalkine and MIP-1; and/or (d) a pharmaceutically acceptable carrier, excipient, or diluent.

In some aspects, the pharmaceutical composition does not contain DMSO. In some aspects, the composition includes further includes a cryopreservation medium, a basal medium, and/or a saline solution. In some aspects, the cryopreservation medium is PRIME-XV® MSC FreeziS DMSO-Free medium. In some aspects, the basal medium is MCDB-131.

In some aspects, the pharmaceutical composition includes about 1×10⁶ to 2.5×10⁶ (e.g., 1.5×10⁶, 1.6×10⁶, 1.7×10⁶, 1.8×10⁶, 1.9×10⁶, 2×10⁶, 2.1×10⁶, 2.2×10⁶, 2.3×10⁶, 2.4×10⁶, or 2.5×10⁶) said UC-MSCs. In some aspects, the pharmaceutical composition includes about 1×10⁶ said UC-MSCs.

In some aspects, the pharmaceutical composition includes a concentration of about 1×10¹⁰ to 5×10¹⁰ (e.g., 1×10¹⁰, 1.5×10¹⁰, 2×10¹⁰, 2.5×10¹⁰, 3×10¹⁰, 3.5×10¹⁰, 4×10¹⁰, 4.5×10¹⁰, or 5×10¹⁰) said isolated exosomes per mL.

In some aspects, the HDAC1 inhibitor is selected from the group including vorinostat, romidepsin, belinostat, panobinostat, valproic acid, entinostat, curcumin, quercetin, and RG2833.

In some aspects, the subject is administered vorinostat in an amount of about 400 mg and/or in an amount sufficient to achieve geometric mean values of a maximum plasma concentration (Cₘₐₓ) and an area under the plasma concentration versus time curve (AUC₀₋ᵢₙₜ) of about 1.2±0.53 μM and about 6.0±2.0 μM*hr, respectively. In some aspects, the subject is administered romidepsin in an amount of about 14 mg/m² IV over a 4-hour period, such as on days 1, 8, and 15 of a 28-day cycle and/or in an amount sufficient to achieve geometric mean values of a maximum plasma concentration (Cₘₐₓ) and an area under the plasma concentration versus time curve (AUC₀₋ᵢₙₜ) of about 377 ng/mL and about 1549 ng*hr/mL, respectively. In some aspects, the subject is administered belinostat in an amount of about 1,000 mg/m² over a 30 minute period, such as on days 1-5 of a 21-day cycle. In some aspects, the subject is administered panobinostat in an amount of about 20 mg every other day, such as on days 1, 3, 5, 8, 10, and 12 of a 21-day cycle. In some aspects, the subject is administered valproic acid in an amount of about 10 to 60 mg/kg/day. In some aspects, the subject is administered entinostat in an amount of about 2 mg/m² to about 12 mg/m² per day. In some aspects, the subject is administered curcumin in an amount of about 1 g to about 8 g per day. In some aspects, the subject is administered quercetin in an amount of about 250 mg to about 1000 mg per day. In some aspects, the subject is administered RG2833 in an amount of about 30 mg to about 240 mg per day.

In some aspects, the composition is administered in a volume of about 0.5 mL to about 15 mL (e.g., 0.5 mL, 0.6 mL, 0.7 mL, 0.8 mL, 0.9 mL, 1 mL, 3 mL, 5 mL, 7 mL, 10 mL, 11 mL, 12 mL, 13 mL, 14 mL, or 15 mL). In some aspects, the composition is administered in a volume of about 1 mL to about 10 mL (e.g., 1 mL, 2 mL, 3 mL, 4 mL, 5 mL, 6 mL, 7 mL, 8 mL, 9 mL, or 10 mL). In some aspects, the composition is administered in a volume of about 1 mL, about 2 mL, about 3 mL, about 4 mL, about 5 mL, or about 6 mL.

In some aspects, the exosomes express CD9, CD63, and CD81. In some aspects, the exosomes further express CD44, CD29, and CD412. In some aspects, the exosomes do not express CD45, CD11b, CD14, CD19, CD34, CD79a, CD126, and HLD-DR.

In some aspects, the pharmaceutical composition includes about 100 pg/mL to about 1000 pg/mL (e.g., 100 pg/mL to 500 pg/mL, 250 pg/mL to 750 pg/mL, or 500 pg/mL to 1000 pg/mL, e.g., 100 pg/mL, 150 pg/mL, 200 pg/mL, 250 pg/mL, 300 pg/mL, 350 pg/mL, 400 pg/mL, 5450 pg/mL, 500 pg/mL, 550 pg/mL, 600 pg/mL, 650 pg/mL, 700 pg/mL, 750 pg/mL, 800 pg/mL, 850 pg/mL, 900 pg/mL, 950 pg/mL, or 1000 pg/mL) of GM-CSF, MIP-3a, IL-6, and IL-8 and about 10 pg/mL to about 100 pg/mL (e.g., 10 pg/mL to 50 pg/mL, 25 pg/mL to 75 pg/mL, or 50 pg/mL to 100 pg/mL, e.g., 10 pg/mL, 20 pg/mL, 30 pg/mL, 40 pg/mL, 50 pg/mL, 60 pg/mL, 70 pg/mL, 80 pg/mL, 90 pg/mL, or 100 pg/mL) of fractalkine and MIP-1. In some aspects, the pharmaceutical composition includes about 1000 μg/mL of GM-CSF, MIP-3a, IL-6, and IL-8 and about 100 μg/mL of fractalkine and MIP-1.

A third aspect of the disclosure features a pharmaceutical composition for use in treating PTHS including: (a) a plurality of UC-MSCs, wherein the UC-MSCs express TCF4; (b) a plurality of isolated exosomes of about 80-200 nm in diameter, wherein the isolated exosomes are derived from the UC-MSCs and express CD9, CD63, CD81, CD44, CD29 and CD142; (c) UC-MSC secretome-conditioned cell culture medium; and/or (d) exosome-depleted UC-MSC-conditioned cell culture medium. In some aspects, the composition further includes one or more Wnt pathway activators.

In some aspects, the Wnt pathway activator is selected from the group including a HDAC1 inhibitor, a Wnt-signaling agonist, a frizzled-signaling agonist, and a GSK-3B13P inhibitor.

In some aspects, the HDAC1 inhibitor is selected from the group including vorinostat, romidepsin, belinostat, panobinostat, valproic acid, entinostat, curcumin, curcumin, quercetin, and RG2833. In some aspects, the Wnt-signaling agonist is L-Quebrachital. In some aspects, the frizzled-signaling agonist is a Wnt agonist-1 protein or a Wnt-3a protein. In some aspects, the GSK-3B13P inhibitor is selected from the groups including indirubin-3′-oxime, laduviglusib (CHIR-99821), and KY19382.

In some aspects, the UC-MSCs are modified to increase expression of TCF4 mRNA and/or protein expression levels relative to unmodified UC-MSCs.

In some aspects, the modified UC-MSCs include a nucleic acid vector, plasmid, circular RNA, or mRNA molecule including a nucleotide sequence encoding a TCF4 protein and/or a frizzled-signaling agonist.

In some aspects, the pharmaceutical composition for use further includes: (a) about 5×10⁵ to 5×10⁶ (e.g., 5×10⁵ to 1×10⁶, 5×10⁵ to 1.5×10⁶, 1×10⁶ to 2.5×10⁶, 1.5×10⁶ to 3×10⁶, 2×10⁶ to 5×10⁶, or 4×10⁶ to 5×10⁶, e.g., 5×10⁵, 6×10⁵, 7×10⁵, 8×10⁵, 9×10⁵, 1×10⁶, 1.5×10⁶, 2×10⁶, 2.5×10⁶, 3×10⁶, 3.5×10⁶, 4×10⁶, 4.5×10⁶, or 5×10⁶) of said UC-MSCs; (b) a concentration of about 5×10⁹ to 5×10¹⁰ (e.g., 5×10⁹ to 1.5×10¹⁰, 1×10¹⁰ to 3×10¹⁰, 2×10¹⁰ to 4×10¹⁰, 3×10¹⁰ to 5×10¹⁰, 1×10¹⁰ to 5×10¹⁰, or 2.5×10¹⁰ to 5×10¹⁰, e.g., 5×10⁹, 6×10⁹, 7×10⁹, 8×10⁹, 9×10⁹, 1×10¹⁰, 1.5×10¹⁰, 2×10¹⁰, 2.5×10¹⁰, 3×10¹⁰, 3.5×10¹⁰, 4×10¹⁰, 4.5×10¹⁰, or 5×10¹⁰) said exosomes per mL; (c) about 100-5000 pg/mL (e.g., 500 pg/mL to 1000 pg/mL, 800 pg/mL to 1600 pg/mL, or 1400 pg/mL to 2000 pg/mL, e.g., 500 pg/mL, 550 pg/mL, 600 pg/mL, 650 pg/mL, 700 pg/mL, 750 pg/mL, 800 pg/mL, 850 pg/mL, 900 pg/mL, 950 pg/mL, 1000 pg/mL, 1050 pg/mL, 1100 pg/mL, 1150 pg/mL, 1200 pg/mL, 1250 pg/mL, 1300 pg/mL, 1350 pg/mL, 1400 pg/mL, 1450 pg/mL, 1500 pg/mL, 1550 pg/mL, 1600 pg/mL, 1650 pg/mL, 1700 pg/mL, 1750 pg/mL, 1800 pg/mL, 1850 pg/mL, 1900 pg/mL, 1950 pg/mL, 2000 pg/mL, 3000 pg/mL, 4000 pg/mL, or 5000 pg/mL) of GM-CSF, MIP-3a, IL-6, and IL-8 and about 10-1000 pg/mL (e.g., 10 pg/mL to 500 pg/mL, 250 pg/mL to 750 pg/mL, or 500 pg/mL to 1000 pg/mL, e.g., 10 pg/mL, 20 pg/mL, 30 pg/mL, 40 pg/mL, 50 pg/mL, 60 pg/mL, 70 pg/mL, 80 pg/mL, 90 pg/mL, 100 pg/mL, 150 pg/mL, 200 pg/mL, 250 pg/mL, 300 pg/mL, 350 pg/mL, 400 pg/mL, 450 pg/mL, 500 pg/mL, 600 pg/mL, 700 pg/mL, 800 pg/mL, 900 pg/mL, or 1000 pg/mL) of fractalkine and MIP-1; and/or (d) a pharmaceutically acceptable carrier, excipient, or diluent.

In some aspects, the pharmaceutical composition for use does not contain DMSO. In some aspects, the pharmaceutical composition for use includes further includes a cryopreservation medium, a basal medium, and/or a saline solution. In some aspects, the cryopreservation medium is PRIME-XV® MSC FreeziS DMSO-Free medium. In some aspects, the basal medium is MCDB-131.

In some aspects, the pharmaceutical composition for use includes about 1×10⁶ to 2.5×10⁶ (e.g., 1.5×10⁶, 1.6×10⁶, 1.7×10⁶, 1.8×10⁶, 1.9×10⁶, 2×10⁶, 2.1×10⁶, 2.2×10⁶, 2.3×10⁶, 2.4×10⁶, or 2.5×10⁶) said UC-MSCs. In some aspects, the pharmaceutical composition includes about 1×10⁶ said UC-MSCs.

In some aspects, the pharmaceutical composition for use includes a concentration of about 1×10¹⁰ to 5×10¹⁰ (e.g., 1×10¹⁰, 1.5×10¹⁰, 2×10¹⁰, 2.5×10¹⁰, 3×10¹⁰, 3.5×10¹⁰, 4×10¹⁰, 4.5×10¹⁰, or 5×10¹⁰) said isolated exosomes per mL.

In some aspects, the HDAC1 inhibitor is selected from the group including vorinostat, romidepsin, belinostat, panobinostat, valproic acid, entinostat, curcumin, quercetin, and RG2833.

In some aspects: (a) vorinostat in an amount of about 400 mg; (b) romidepsin in an amount of about 14 mg/m² of a subject's body surface area; (c) belinostat in an amount of about 1,000 mg/m² of the subject's body surface area; (d) panobinostat in an amount of about 20 mg; (e) valproic acid in an amount of about 10 to 60 mg/kg of the subject's body weight; (f) entinostat in an amount of about 2 mg/m² to about 12 mg/m² of the subject's body surface area; (g) curcumin in an amount of about 1 g to about 8 g; (h) quercetin in an amount of about 250 mg to about 1000 mg; and/or (i) RG2833 in an amount of about 30 mg to about 240 mg.

In some aspects, the pharmaceutical composition for use is administered in a volume of about 0.5 milliliters (mL) to about 15 mL (e.g., 0.5 mL, 0.6 mL, 0.7 mL, 0.8 mL, 0.9 mL, 1 mL, 3 mL, 5 mL, 7 mL, 10 mL, 11 mL, 12 mL, 13 mL, 14 mL, or 15 mL). In some aspects, the pharmaceutical composition for use is administered in a volume of about 1 mL to about 10 mL (e.g., 1 mL, 2 mL, 3 mL, 4 mL, 5 mL, 6 mL, 7 mL, 8 mL, 9 mL, or 10 mL). In some aspects, the pharmaceutical composition for use is administered in a volume of about 1 mL, about 2 mL, about 3 mL, about 4 mL, about 5 mL, or about 6 mL.

In some aspects, the exosomes express CD9, CD63, and CD81. In some aspects, the exosomes further express CD44, CD29, and CD412. In some aspects, the exosomes do not express CD45, CD11b, CD14, CD19, CD34, CD79a, CD126, and HLD-DR.

In some aspects, the pharmaceutical composition for use includes about 100 μg/mL to about 1000 pg/mL (e.g., 100 pg/mL to 500 pg/mL, 250 pg/mL to 750 pg/mL, or 500 pg/mL to 1000 pg/mL, e.g., 100 pg/mL, 150 pg/mL, 200 pg/mL, 250 pg/mL, 300 pg/mL, 350 pg/mL, 400 pg/mL, 5450 pg/mL, 500 pg/mL, 550 pg/mL, 600 pg/mL, 650 pg/mL, 700 pg/mL, 750 pg/mL, 800 pg/mL, 850 pg/mL, 900 pg/mL, 950 pg/mL, or 1000 pg/mL) of GM-CSF, MIP-3a, IL-6, and IL-8 and about 10 pg/mL to about 100 pg/mL (e.g., 10 pg/mL to 50 pg/mL, 25 pg/mL to 75 pg/mL, or 50 pg/mL to 100 pg/mL, e.g., 10 pg/mL, 20 pg/mL, 30 pg/mL, 40 pg/mL, 50 pg/mL, 60 pg/mL, 70 pg/mL, 80 pg/mL, 90 pg/mL, or 100 pg/mL) of fractalkine and MIP-1. In some aspects, the pharmaceutical composition for use includes about 1000 pg/mL of GM-CSF, MIP-3a, IL-6, and IL-8 and about 100 pg/mL of fractalkine and MIP-1.

### BRIEF DESCRIPTION OF THE DRAWINGS

The accompanying drawings are included to illustrate embodiments of the disclosure and further an understanding of its implementations.

FIG. 1 is a schematic showing an overview of the umbilical cord-derived mesenchymal stem cell (UC-MSC) manufacturing process. Shown are the various procedures used in the manufacture and quality control of UC-MSCs (e.g., ALLORX STEM CELLS®). Collection of the donor umbilical cord is shown at the top and also described herein. In the block labeled “Good Manufacturing Practice Conditions”, processing of the umbilical cord is shown followed by purification of MSCs in Pass 0. The Master Cell Bank is the cryopreserved cells from Pass 0. Subculture of these cells occurs through multiple passages each of which becomes working cell banks until the “Bulk cellular dose” is generated. This is the bulk Active Pharmaceutical Ingredient (API) then subject to fill/finish procedures (e.g., “Cryopreserving, packing and labeling”) that becomes the final cellular medicine after completion of all quality control procedures and achieving quality control release criteria. Retention samples are used for future testing as may be required by the quality management system.

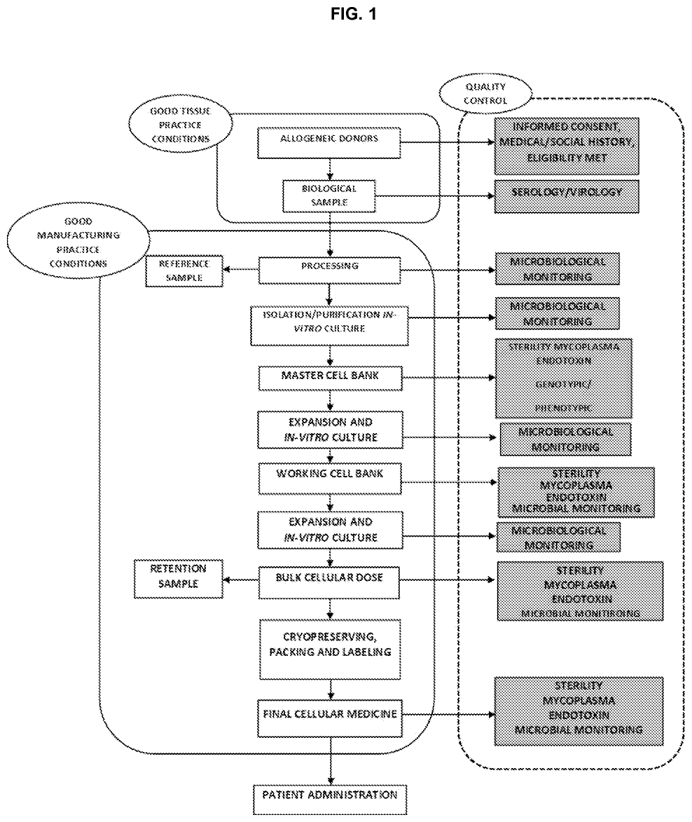

FIG. 2 is a diagram showing diafiltration of active cell culture in a stirred tank bioreactor to exchange the cell culture growth medium with basal medium as described in Example 1.

FIG. 3 is a diagram showing the UC-MSC secretome-conditioned cell culture medium harvesting step. This step occurs after an additional period of 72 hours in culture within the bioreactor following diafiltration (e.g., see FIG. 2). Harvesting of UC-MSC secretome-conditioned cell culture medium is further described in Example 1.

FIG. 4 is a series of photographs showing tri-lineage differentiation of UC-MSCs. Cellular markers are used to demonstrate UC-MSC differentiation into bone, cartilage and fat cells.

FIG. 5 shows an example of a Certificate of Analysis for manufactured UC-MSCs (e.g., ALLORX STEM CELLS®), as described herein.

FIG. 6 is a set of tables and graphs showing the results of an analysis of exosome-depleted UC-MSC-conditioned cell culture medium. Exosomes contained in the secretome-conditioned cell culture medium were purified by size exclusion chromatography as described in Example 3. In brief, the exosomes were stained with CD63-AF488, CD9-AF488, and CD81-DYLight550 antibodies and analyzed by FACS. % CD=antibody fluorescence/scatter×100. The liposomes are used as a standard to verify labeling by the unspecific, lipophilic membrane dye CellMask™ Deep Red (CDMR). Purity=CMDR fluorescence/scatter×100. VBP=SEC purified ALLOEX EXOSOMES®. EVs=size exclusion purified exosomes.

FIG. 7 is a graph showing the secretion of heat shock protein 70 (Hsp70) from cultured UC-MSCs (e.g., ALLORX STEM CELLS®) and the effects of increasing concentrations of lithium chloride (LiCl) on Hsp70 secretion.

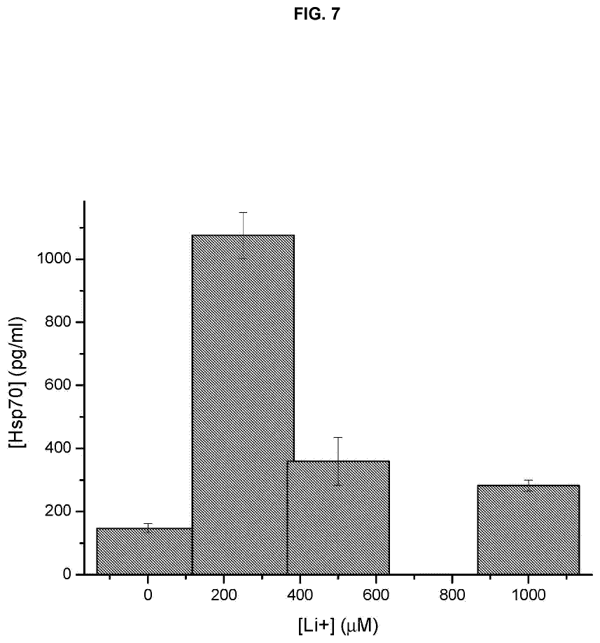

FIG. 8. is a set of photographs showing an exemplary method for preparing cryopreserved UC-MSCs (e.g., ALLORX STEM CELLS®) for injection. The UC-MSCs are shipped in cryogenic conditions and require storage in liquid nitrogen prior to use. To prepare the UC-MSCs for administration to a subject (e.g., a subject with Pitt-Hopkins Syndrome (PTHS)), first thaw the cells at 37° C. immediately after being taken from the dewar using a water bath previously equilibrated to 37° C. Second, continuously swirl the vial of cells while submerged in water bath until they are thawed. Generally, it takes about 5 minutes to completely thaw the contents of a 5 ml vial. Third, in a sterile environment, use a sterile syringe with an attached sterile needle (e.g., an 18 gauge needle) to pull the cells out of the cryogenic vial. Change the needle before slowly pushing the cells into an IV bag to maintain cellular viability. Infuse the cells at about 50 drops per minute using a 100 ml normal saline drip. Any additional preparation step(s), such as pretreatment of the UC-MSCs, may be employed as described in the detailed description.

FIG. 9 shows growth and expansion characteristic of AD-MSCs, BM-MSCs, P-MSCs and UCMSCs following pass 2 in cell culture. Black bars are cell count, red bars are doubling times: T In (Cf-Ci)/Ci where T is the time from subculture to detachment (Hrs), Ci is the initial cell count and Cf is the final cell count and blue bars are PI-determined viability.

FIG. 10 shows immunomodulatory potency of UC-MSCs, AD-MSCs, P-MSCs and UC-MSCs by an γ-IFN induced IDO activity assay.

FIG. 11 illustrates that UC-MSCs have a significantly higher cellular ATP-content than the other ADMSCs, P-MSCs, & BM-MSCs.

FIG. 12 shows a comparison of migration by AD-MSCs, P-MSCs, BM-MSCs & UC-MSCs into cell-free regions. Migration was determined as described in the Materials and Methods and the measured % closure of the occluded region is plotted as a function of time after exposure to 50 μg/ml Substance P.

FIG. 13 shows the proliferation of AD-MSCs, P-MSCs, BM-MSCs and UC-MSCs in varying levels of FBS added to a serum-free base. RFU's at day 3 minus day 1 following Presto Blue exposure are shown as a function of [FBS].

FIG. 14 shows the IHC results of the staining for GLAST in P-MSCs, AD-MSCs, UC-MSCs and the control NSCs. While the cells differentiated from the UC-MSCs and the control NSCs were positive for GLAST, the AD-MSCs and P-MSCs were not. The apparent difference between the UC-MSC-derived cells and the control NSCs is due to cell density, both are positive for GLAST.

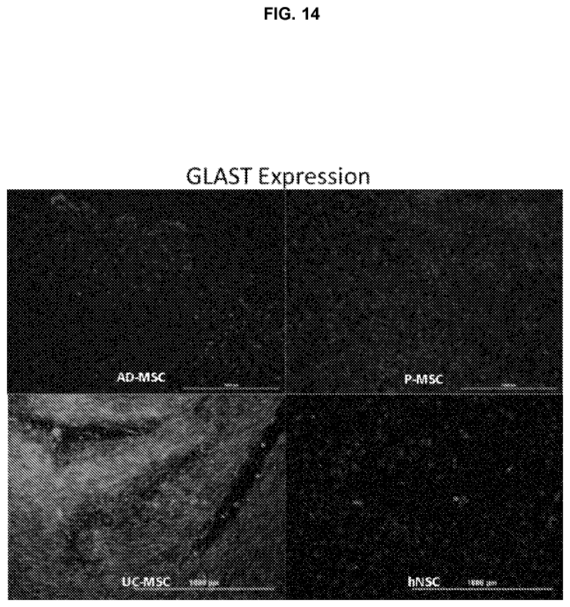

### DEFINTIONS

Unless otherwise defined herein, scientific, and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

As used herein, the term “about,” as applied to one or more values of interest, refers to a value that falls within 10%, 9%, 8%, 7%, 6%, 5%, 4%, 3%, 2%, 1%, or less in either direction (greater than or less than (+/−)) of a stated reference value, unless otherwise stated or otherwise evident from the context (except where such number would exceed 100% of a possible value).

As used herein, the term “exosome-depleted UC-MSC-conditioned cell culture medium” refers to UC-MSC secretome-conditioned cell culture medium that has been processed to remove exosomes (e.g., exosomes that are of a specific size, e.g., 80-200 nm). Several standard laboratory techniques exist to remove exosomes from cell culture medium, such as differential ultracentrifugation, size exclusion chromatography, ultrafiltration, polyethylene glycol-based precipitation, immunoaffinity capture, or by using microfluidic devices. Exosome-depleted UC-MSC-conditioned cell culture medium may contain other UC-MSC-derived biological material, such as proteins (e.g., TCF4), lipids, and extracellular vesicles (EVs) smaller than 80 nm or larger than 200 nm.

As used herein, the term “increased expression” refers to an expression level of an mRNA or protein (e.g., TCF4 mRNA or protein) that is at least 5% higher (e.g., 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500% or more) than a control (e.g., the expression level of the mRNA or protein (e.g., TCF4) in an untreated cell (e.g., an untreated UC-MSC) or in an untreated subject), as determined by an objective assay (e.g., PCR, RT-PCR, qPCR, RT-qPCR, microarray analysis, Northern blot, MASSARRAY® technique, SAGE, RNA-sequencing, flow cytometry (FC), fluorescence-activated cell sorting (FACS) Western blot, enzyme-linked immunosorbent assay (ELISA), mass spectrometry (MS), immunofluorescence (IF), immunoprecipitation (IP), radioimmunoassay, dot blotting, high performance liquid chromatography (HPLC), surface plasmon resonance, optical spectroscopy, and immunohistochemistry (IHC)).

As used herein, the term “isolated exosome” refers to an exosome (or a population of exosomes) that was isolated from UC-MSC secretome-conditioned cell culture medium. The isolated exosomes are 80 nm to 200 nm (e.g., 80 nm, 90 nm, 100 nm, 110 nm, 120 nm, 130 nm, 140 nm, 150 nm, 160 nm, 170 nm, 180 nm, 190 nm, or 200 nm) in diameter and express cluster of differentiation (CD)-9, CD63, CD81, CD44, CD29, and CD142. Further, isolated exosomes do not express CD45, CD11b, CD14, CD19, CD34−, CD79a, CD126, and human leukocyte antigen-DR isotype (HLD-DR).

As used herein, the terms “umbilical cord-derived human mesenchymal stem cell” and “UC-MSC” refer to a class of multifunctional stem cells isolated and cultured from umbilical cord. They are capable of self-renewal, tri-lineage differentiation potential, and low immunogenicity.

As used herein, the terms “UC-MSC secretome-conditioned cell culture medium” and “secretome-conditioned cell culture medium” refer to cell culture medium that was previously incubated with a plurality of UC-MSCs. As a result, the cell culture medium comprises biological material (e.g., proteins (e.g., TCF4), exosomes, and lipids that may have been secreted from the UC-MSCs.

As used herein, the term “Wnt pathway activator” refers to any compound (e.g., chemical, small molecule, peptide, protein, or protein complex) that can stimulate or activate the Wnt signaling pathway. Wnt pathway activators include, but are not limited to, histone deacetylase 1 (HDAC1) inhibitors (e.g., vorinostat, romidepsin, belinostat, panobinostat, valproic acid, entinostat, curcumin, quercetin, and RG2833), Wnt-signaling agonists (e.g., L-Quebrachital), frizzled-signaling agonists (e.g., Wnt agonist-1 or Wnt-3a), and glycogen synthase kinase-3p (GSK-3B13p) inhibitors (e.g., indirubin-3′-oxime, landuviglusib (CHIR-99821), and KY19382).

### DETAILED DESCRIPTION

Embodiments of the disclosure described herein comprise efficacy of MSC therapy in PTHS patients by cell-based enhanced proliferation of neural progenitor cells, regeneration of damaged CNS as well as a therapy involving cellular and gene therapy replacement of the normal haplotype of the TCF4 gene. Other mechanisms involve neural cell protection, immunomodulation, and anti-inflammatory effects. While iPSCs have been generated from PTHS patients (Sripathy, S R, et al, Stem Cell Res 48: 102001, 2020) we are not aware of prior clinical testing of MSC therapy for PTHS patients. However, evidence of the prospect of direct benefit to PTHS patients has been obtained: 1.) ASD patients have been subject to MSC-based clinical studies, typically Phase I/I1 study design without randomization, placebo control or double-blinding. Two studies used IV infusion of MSCs for intervention, one was a total of 144 million MSCs in 4 equal doses over a nine month time course; another used four MSC/CB MNC IV infusions or IT injections once a week for 4 weeks at 2×10⁶ CB-MNC/kg and 1×10⁶ CB-MSC/kg; a total of 57 patients were in these studies. Neither study (Riordan, N H, et al, Stem Cells Translational Medicine 8: 1008-1016, 2019; Lv, Y T, et al, J. of Translational Medicine 11: 196-206, 2013) reported significant adverse events; AEs were mild to moderate and short in duration supporting safety of MSC therapy of ASD patients. Various efficacy endpoints were used including The Childhood Autism Rating Scale (CARS), Clinical Global Impression (CGI) scale, Aberrant Behavior Checklist (ABC) and Autism Treatment Evaluation Checklist (ATEC). In addition, one study evaluated the levels of two cytokines (MDC & TARC) pre- and post-treatment. Both studies reported reductions in behavior-based test scores following treatment with one (Lv, Y T, et al, J. of Translational Medicine 11: 196-206, 2013) reporting statistically significant different reduction in CARS, ABC scores and CGI scale in treatment groups 24 weeks post-treatment (p<0.05). Thus, these studies provide evidence of safety and efficacy of MSC therapy of ASD patients and since PTHS patients exhibit ASD symptoms these results support the prospect of direct benefit by treatment of PTHS patients with ALLORX STEM CELLS®.

2.) Further clinical studies of MSCs relevant to our proposed study include use of MSC therapy of stroke. An earlier Phase I/IIa study at Stanford (Steinberg, G K, et al, Stroke 47: 1817-1824, 2016) involved stereotactic injection into infarcted brain regions showing no differences in dosages from 2.5, 5 or 10 million BM-MSCs transiently transfected with the Notch gene, rendering differentiation into Neural Stem Cells. The trial involved 18 patients, 16 showed significant improvement in several stroke scales, including the National Institutes of Health Scale. Adverse events were determined to be related to injection procedures rather than the MSCs (Steinberg, G K, et al, Stroke 47: 1817-1824, 2016). These results were extended by showing apparent safety and efficacy of IV MSC infusion for chronic stroke, without apparent dosage dependency at 0.5, 1.0 and 2.0 million BM-MSCs/kg (Levy, M L, et al, Stroke 50: 2835-2841, 2019). These studies are also supported by pre-clinical results described in greater detail below.

3) Evidence of the prospect of direct benefit is also supported by evidence of safety and efficacy of MSC therapy in pediatric populations of children with Cerebral Palsy (Huang, L, et al, Cell Transplantation 27: 325-334, 2018 “A Randomized, Placebo-Controlled Trial of Human Umbilical Cord Blood Mesenchymal Stem Cell Infusion for Children With Cerebral Palsy”-https://www.ncbi.nlm.nih.gov/pmc/articles/PMC5898688). All 54 patients received basic rehabilitation as a background treatment. The infusion group comprising 27 patients received 4 infusions of human umbilical cord-derived MSCs (UC-MSCs) intravenous infusions at a fixed dose of 50 million cells) and basic rehabilitation treatment, whereas 27 patients in the control group received 0.9% normal saline and basic rehabilitation treatment. Several indices were tested from baseline up to 24 months posttreatment regarding efficacy and safety evaluations, including the gross motor function measurement 88 (GMFM-88) scores, the comprehensive function assessment (CFA), lab tests, electroencephalogram (EEG), routine magnetic resonance imaging (MRI), and adverse events. The changes in the total proportion of GMFM-88 and total scores of CFA in the hUCB-MSC infusion group were significantly higher than that in control group at 3, 6, 12, 24 months posttreatment (All p values were=0.000 by T-test analysis)

Less diffuse slow waves were noticed after hUCB-MSC infusion in patients with slowing of EEG back-ground rhythms at baseline. Based on the routine MRI exams, improvements in cerebral structures were rare after treatment. Serious adverse events were not observed during the whole study period. The results of the study indicated that hUCB-MSC infusion with basic rehabilitation was safe and effective in improving gross motor and comprehensive functions in children with CP. Individuals with Pitt Hopkins (PTHS) are delayed in their motor milestones and often present early with hypotonia, but over time develop hypertonia, decreased range of motion and crouch gait similar to the course seen in individuals with cerebral palsy. Given the prior results in the CP studies, our outcome measures will include gross motor functional assessment as the data from this CP trials suggests UC-MSCs to be efficacious.

Disclosed herein are methods of therapy of Pitt-Hopkins Syndrome (PTHS) involving both cell and gene therapy by treatment using umbilical cord (UC)-derived human mesenchymal stem cells (MSCs), their non-cellular derivatives (e.g., the UC-MSC secretome and exosomes isolated therefrom) and various molecular compositions to supplement or increase expression of the transcription factor 4 (TCF4) gene, thereby restoring or increasingTCF4 function in a subject with PTHS. The cellular and non-cellular therapies described herein can be used to alleviate or reduce damage caused by TCF4 gene mutations or deletions by restoring neurogenesis and correcting damaged neural tissue by, e.g., a reduction of apoptosis, anti-oxidative effects, angiogenesis, correction of DNA damage, and/or reduction of MMP enzyme induction. Gene therapy is provided by supplementing the TCF4 gene or TCF4 protein activity in a subject with PTHS (e.g., by increasing expression of a functional TCF4 gene) by use of therapeutic cells (e.g., UC-MSCs) containing a fully functional TCF4 gene. The disclosure also provides methods to increase TCF4 gene expression in the transplanted stem cells (e.g., UC-MSCs).

Based on a comparative study, we have discovered that mesenchymal stem cells (MSCs) derived from umbilical cord (UC)-MSCs (UC-MSCs), exhibit superior performance over MSCs derived from other human tissues. We have developed commercial biomanufacturing of clinical grade umbilical cord derived MSCs (e.g., ALLORX STEM CELLS®). These MSCs have been used in over 300 deployments by intravascular (IV) infusion, intra-articular (IA), intramuscular (IM) and intrathecal (IT) injections in IRB-approved, “Right- to-Try” and FDA authorized elNDs (compassionate use INDs) clinical trials for various indications without severe adverse events (SAE) and with evidence of efficacy. Those adverse events (AE) occurring were temporary and resolved spontaneously and have mainly been associated with IA or IT modes of administration.

We have evidence showing that neurodegenerative diseases including multiple sclerosis (MS) have been treated by IV infusion of our UC-MSCs, showing that this form of administration is safe and has yielded evidence of efficacy in the treatment of MS.

A typical embodiment in accordance with this disclosure involves gene as well as cell therapy of PTHS by the therapeutic use of UC-derived MSCs with or without the MSC secretome or any of its components. MSCs are known to pass the blood brain barrier (BBB) in an in-vitro model of the BBB consisting of rat brain microvascular endothelial cells (BMEC) and BM-MSCs in cell culture inserts. There was decreased barrier function at 1.5×10⁵ MSCs/cm² with slower disruption at 1.5×10⁴ MSCs/cm². MSCs were found in the subendothelial space suggesting transmigration across the BMEC monolayer (T. Matsushita, et al., Mesenchymal stem cells transmigrate across brain microvascular endothelial cell monolayers through transiently formed inter-endothelial gaps. Neurosci Letts 502: 41-45, 2011). It is also important to consider the status of the BBB in specific neurodegenerative conditions. These are often characterized by neuroinflammation that induces disruption of the barrier properties of the BBB. For example, in post-mortem studies of ASD patients compared to schizophrenic and healthy subjects with regard to tight junctions, other key molecules associated with neurovascular unit integrity were investigated. Pore forming genes, Claudin (CLDN-5 and CLDN-12) were increased in the ASD cortex and cerebellum. Barrier forming tight junction components (CLDN-1, OCLN and TRIC) showed decreased expression in the intestines of ASD patients. Since PTHS patients exhibit ASD symptoms it is likely that the underlying neuroinflammation alters BBB function thus allowing enhanced transfer of ALLORX STEM CELLS® across the BBB in PTHS patients compared to a healthy study population. (Fiorentino, M, et al., Blood-brain barrier and intestinal epithelial barrier alterations in autism spectrum disorders. Molecular Autism 7: 49-66, 2016). Alterations of BBB function have been reported in cerebral palsy (CP), Huntington's disease (HD) and MS (Stolp, H B and Dziegielewska, KM, Review: Role of developmental inflammation and blood-brain barrier dysfunction in neurodevelopmental and neurodegenerative diseases. Neuropathol. Appl. Neurobiol. 35:132-146, 2009; Sweeney, M D, et al., Blood-brain barrier breakdown in Alzheimer's disease and other neurodegenerative disorders. Nat Rev Neurol 14: 133-150, 2018; Huang, L, et al, A randomized, placebo-controlled trial of human umbilical cord blood mesenchymal stem cell infusion for children with cerebral palsy. Cell Transplantation 27: 325-334, 2018; HD: Barros, I, et al., Mesenchymal stromal cells' therapy for polyglutamine disorders: Where do we stand and where should we go?Front Cell Neuroscience 14: 1-26, 2020; MS: Riordan, N H, et al, Clinical feasibility of umbilical cord tissue-derived mesenchymal stem cells in the treatment of multiple sclerosis. J Transl Med 16: 57-69, 2018. Thus, during treatment of PTHS patients with ALLORX STEM CELLS® by IV infusion the cells would cross the BBB and enter the cerebral spinal fluid, and since the ALLORX STEM CELLS® express TCF4 without mutation because of the extensive screening of umbilical cords used in manufacturing, TCF4 would be expressed in the PTHS patients. A preferred embodiment of the disclosure is that ALLORX STEM CELLS® will also induce gene therapy by replacement of the function of TCF4.

In some embodiments, the UC-MSCs can be co-administered with other known treatments for PTHS. For example, a patient can receive standard of care palliative treatments (e.g., behavior medications, physical therapy, behavioral therapy, speech therapy, occupational therapy, and nutritional counseling) and UC-MSCs. Also, the treatment may involve use of UC-MSCs that have been contacted with an HDAC1 inhibitor(s) or Wnt-3a (or a Wnt pathway activator) prior to administration (e.g., transplantation) to a PTHS subject, e.g., to increase TCF4 expression in the transplanted UC-MSCs. In addition, the UC-MSCs used for treatment may be genetically engineered to increase expression of TCF4.

Embodiments herein include methods for treating a patient with PTHS with cellular (e.g., UC-MSCs) and/or non-cellular (e.g., exosomes or a secretome derived from, e.g., UC-MSCs). Treatments include administering human UC-MSCs, and in some cases, stem cells known as ALLORX STEM CELLS® to the patient. Cells are typically delivered via IV, intranasally, or intrathecally, although other known delivery routes are envisioned. From about 1 to 2.5 million cells/kg body weight are delivered per treatment, particularly when the cells are administered by IV. Other numbers of cells can be administered, as long as the number provides a benefit to the patient (a benefit being any reduction in a symptom of PTHS, as discussed below).

Methods herein include optionally identifying whether a patient has PTHS. A dose of human MSCs can then be determined for a patient determined to have PTHS based on the weight of the patient. Once a dose is identified, a mode of administration is determined, such as IV. The predetermined dose is then administered to the patient over an appropriate period of time. In an alternative embodiment, the human UC-MSCs are pretreated to increase TCF4 expression prior to administration to the patient. In some aspects, the cells can be pretreated with a Wnt receptor ligand or other agent described herein. Alternatively, the patient can be pretreated or post-treated with these same agents to increase TCF4 expression in conjunction with cell (and/or non-cellular) administration. Treatments herein can be repeated over the course of weeks, months, or years, and until one or more of the PTHS symptoms is improved or the disease itself if marginalized.

### 1. Methods of Treating PTHS

The methods of treating PTHS in a subject in need thereof utilize compositions that contain an effective amount of UC-MSCs, isolated exosomes derived from the UC-MSCs, cell culture medium containing the UC-MSC's secretome (e.g., UC-MSC secretome-conditioned cell culture medium), cell culture medium containing the UC-MSC's secretome but devoid of exosomes (e.g., exosome-depleted UC-MSC-conditioned cell culture medium), or some combination thereof.

A method of treating a subject with PTHS may include administering to the subject an effective amount of: (a) a plurality of umbilical cord-derived human mesenchymal stem cells (UC-MSCs, such as ALLORX STEM CELLS®), in which express functional transcription factor 4 (TCF4); (b) a plurality of isolated exosomes of about 80-200 nanometers (nm) in diameter, in which are derived from the UC-MSCs; (c) UC-MSC secretome-conditioned cell culture medium; and/or (d) exosome-depleted UC-MSC-conditioned cell culture medium. With regards to compositions containing UC-MSCs expressing TCF4, the methods of treatment described herein may further include, as an option, increasing TCF4 expression (e.g., TCF4 mRNA and/or protein production) by the administered UC-MSC (either prior to administration or post-administration in the body of the treated subject). Details on these are described in Section I below. Further details on each of the administered compositions are described below.

### A. Administration

A composition containing about 5×10⁵ to 5×10⁶ (e.g., 5×10⁵ to 1×10⁶, 5×10⁵ to 1.5×10⁶, 1×10⁶ to 2.5×10⁶, 1.5×10⁶ to 3×10⁶, 2×10⁶ to 5×10⁶, or 4×10⁶ to 5×10⁶, e.g., 5×10⁵, 6×10⁵, 7×10⁵, 8×10⁵, 9×10⁵, 1×10⁶, 1.5×10⁶, 2×10⁶, 2.5×10⁶, 3×10⁶, 3.5×10⁶, 4×10⁶, 4.5×10⁶, or 5×10⁶) of the UC-MSCs may be administered to a subject having PTHS. For example, a composition containing 1×10⁶ to 2.5×10⁶ (e.g., 1.5×10⁶, 1.6×10⁶, 1.7×10⁶, 1.8×10⁶, 1.9×10⁶, 2×10⁶, 2.1×10⁶, 2.2×10⁶, 2.3×10⁶, 2.4×10⁶, or 2.5×10⁶) of the UC-MSCs may be administered to the subject having PTHS. In another example, a composition containing about 1×10⁶ (e.g., 9×10⁵, 1×10⁶, or 1.1×10⁶) of the UC-MSCs is administered to the subject having PTHS. UC-MSCs may be isolated from the subject having PTHS or isolated from a healthy donor (e.g., a subject that does not have PTHS). UC-MSCs may be allogenic or autologous.

A composition containing a concentration of about 5×10⁹ to 5×10¹⁰ (e.g., 5×10⁹ to 1.5×10¹⁰, 1×10¹⁰ to 3×10¹⁰, 2×10¹⁰ to 4×10¹⁰, 3×10¹⁰ to 5×10¹⁰, 1×10¹⁰ to 5×10¹⁰, or 2.5×10¹⁰ to 5×10¹⁰, e.g., 5×10⁹, 6×10⁹, 7×10⁹, 8×10⁹, 9×10⁹, 1×10¹⁰, 1.5×10¹⁰, 2×10¹⁰, 2.5×10¹⁰, 3×10¹⁰, 3.5×10¹⁰, 4×10¹⁰, 4.5×10¹⁰, or 5×10¹⁰) of the isolated exosomes per mL may be administered to a subject having PTHS. A composition containing about 100 μg to about 300 pg (e.g., 100 μg to 200 μg, 125 μg to 175 pg, or 200 μg to 300 pg, e.g., 100 μg, 125 pg, 150 μg, 175 pg, 200 μg, 225 pg, 250 μg, 275 pg, or 300 pg) of the isolated exosomes may be administered to a subject having PTHS. The isolated exosomes may be derived from US-MSC secretome-conditioned cell culture medium. After removing exosomes from US-MSC secretome-conditioned cell culture medium, exosomes may contain a volume (e.g., a volume of about 2.7×10⁻¹⁰ mm³ or less) of the cell culture medium from which they were isolated from; this is still considered an “isolated” exosome.

A composition containing US-MSC secretome-conditioned cell culture medium may include about 100 pg/mL to 2000 pg/mL (e.g., 500 pg/mL to 1000 pg/mL, 800 pg/mL to 1600 pg/mL, or 1400 pg/mL to 2000 pg/mL, e.g., 500 pg/mL, 550 pg/mL, 600 pg/mL, 650 pg/mL, 700 pg/mL, 750 pg/mL, 800 pg/mL, 850 pg/mL, 900 pg/mL, 950 pg/mL, 1000 pg/mL, 1050 pg/mL, 1100 pg/mL, 1150 pg/mL, 1200 pg/mL, 1250 pg/mL, 1300 pg/mL, 1350 pg/mL, 1400 pg/mL, 1450 pg/mL, 1500 pg/mL, 1550 pg/mL, 1600 pg/mL, 1650 pg/mL, 1700 pg/mL, 1750 pg/mL, 1800 pg/mL, 1850 pg/mL, 1900 pg/mL, 1950 pg/mL, 2000 pg/mL) of granulocyte-macrophage colony-stimulating factor (GM-CSF), macrophage inflammatory protein (MIP)-3 alpha (MIP-3a), interleukin (IL)-6, and IL-8 and may be administered to a subject having PTHS. For example, a composition containing about 100 pg/mL to 1000 pg/mL (e.g., 100 pg/mL to 500 pg/mL, 250 pg/mL to 750 pg/mL, or 500 pg/mL to 1000 pg/mL, e.g., 100 pg/mL, 150 pg/mL, 200 pg/mL, 250 pg/mL, 300 pg/mL, 350 pg/mL, 400 pg/mL, 5450 pg/mL, 500 pg/mL, 550 pg/mL, 600 pg/mL, 650 pg/mL, 700 pg/mL, 750 pg/mL, 800 pg/mL, 850 pg/mL, 900 pg/mL, 950 pg/mL, or 1000 pg/mL) of GM-CSF, MIP-3a, IL-6, and IL-8 in US-MSC secretome-conditioned cell culture medium may be administered to a subject having PTHS. The composition may further include about 10 pg/mL to 500 pg/mL (e.g., 10 pg/mL to 100 pg/mL, 10 pg/mL to 500 pg/mL, or 500 pg/mL to 1000 pg/mL, e.g., 10 pg/mL, 20 pg/mL, 30 pg/mL, 40 pg/mL, 50 pg/mL, 60 pg/mL, 70 pg/mL, 80 pg/mL, 90 pg/mL, 100 pg/mL, 150 pg/mL, 200 pg/mL, 250 pg/mL, 300 pg/mL, 350 pg/mL, 400 pg/mL, 450 pg/mL, or 500 pg/mL) of fractalkine and MIP-1 that is to be administered to the subject. For example, a composition containing US-MSC secretome-conditioned cell culture medium may include about 100 pg/mL to 1000 pg/mL of GM-CSF, MIP-3a, IL-6, and IL-8, and about 10 pg/mL to about 100 pg/mL (e.g., 10 pg/mL to 50 pg/mL, 25 pg/mL to 75 pg/mL, or 50 pg/mL to 100 pg/mL, e.g., 10 pg/mL, 20 pg/mL, 30 pg/mL, 40 pg/mL, 50 pg/mL, 60 pg/mL, 70 pg/mL, 80 pg/mL, 90 pg/mL, or 100 pg/mL) of fractalkine and MIP-1. These concentration ranges are envisioned per protein.

A composition containing exosome-depleted US-MSC-conditioned cell culture medium may include about 100 pg/mL to 2000 pg/mL (e.g., 500 pg/mL to 1000 pg/mL, 800 pg/mL to 1600 pg/mL, or 1400 pg/mL to 2000 pg/mL, e.g., 500 pg/mL, 550 pg/mL, 600 pg/mL, 650 pg/mL, 700 pg/mL, 750 pg/mL, 800 pg/mL, 850 pg/mL, 900 pg/mL, 950 pg/mL, 1000 pg/mL, 1050 pg/mL, 1100 pg/mL, 1150 pg/mL, 1200 pg/mL, 1250 pg/mL, 1300 pg/mL, 1350 pg/mL, 1400 pg/mL, 1450 pg/mL, 1500 pg/mL, 1550 pg/mL, 1600 pg/mL, 1650 pg/mL, 1700 pg/mL, 1750 pg/mL, 1800 pg/mL, 1850 pg/mL, 1900 pg/mL, 1950 pg/mL, 2000 pg/mL) of GM-CSF, MIP-3a, IL-6, and IL-8 and may be administered to a subject having PTHS. For example, a composition containing about 100 pg/mL to 1000 pg/mL (e.g., 100 pg/mL to 500 pg/mL, 250 pg/mL to 750 pg/mL, or 500 pg/mL to 1000 pg/mL, e.g., 100 pg/mL, 150 pg/mL, 200 pg/mL, 250 pg/mL, 300 pg/mL, 350 pg/mL, 400 pg/mL, 5450 pg/mL, 500 pg/mL, 550 pg/mL, 600 pg/mL, 650 pg/mL, 700 pg/mL, 750 pg/mL, 800 pg/mL, 850 pg/mL, 900 pg/mL, 950 pg/mL, or 1000 pg/mL) of GM-CSF, MIP-3a, IL-6, and IL-8 in exosome-depleted US-MSC-conditioned cell culture medium may be administered to a subject having PTHS. The composition may further include about 10 pg/mL to 500 pg/mL (e.g., 10 pg/mL to 100 pg/mL, 10 pg/mL to 500 pg/mL, or 500 pg/mL to 1000 pg/mL, e.g., 10 pg/mL, 20 pg/mL, 30 pg/mL, 40 pg/mL, 50 pg/mL, 60 pg/mL, 70 pg/mL, 80 pg/mL, 90 pg/mL, 100 pg/mL, 150 pg/mL, 200 pg/mL, 250 pg/mL, 300 pg/mL, 350 pg/mL, 400 pg/mL, 450 pg/mL, or 500 pg/mL) of fractalkine and MIP-1 that is to be administered to the subject.

For example, a composition containing exosome-depleted US-MSC-conditioned cell culture medium may include about 100 pg/mL to 1000 pg/mL of GM-CSF, MIP-3a, IL-6, and IL-8, and about 10 pg/mL to about 100 pg/mL (e.g., 10 pg/mL to 50 pg/mL, 25 pg/mL to 75 pg/mL, or 50 pg/mL to 100 pg/mL, e.g., 10 pg/mL, 20 pg/mL, 30 pg/mL, 40 pg/mL, 50 pg/mL, 60 pg/mL, 70 pg/mL, 80 pg/mL, 90 pg/mL, or 100 pg/mL) of fractalkine and MIP-1. These concentration ranges are envisioned per protein.

Any of the compositions described herein (e.g., a composition containing a plurality of UC-MSCs, a plurality of isolated exosomes, US-MSC secretome-conditioned cell culture medium, exosome-depleted UC-MSC-conditioned cell culture medium, or some combination thereof) may be administered to a subject having PTHS in a volume of about 0.5 milliliters (mL) to about 15 mL (e.g., 0.5 mL, 0.6 mL, 0.7 mL, 0.8 mL, 0.9 mL, 1 mL, 3 mL, 5 mL, 7 mL, 10 mL, 11 mL, 12 mL, 13 mL, 14 mL, or 15 mL), about 1 mL to about 10 mL (e.g., 1 mL, 2 mL, 3 mL, 4 mL, 5 mL, 6 mL, 7 mL, 8 mL, 9 mL, or 10 mL), or about 1 mL to about 6 mL, (e.g., 1 mL, 1.25 mL, 1.5 mL, 1.75 mL, 2 mL, 2.25 mL, 2.5 mL, 2.75 mL, 3 mL, 3.25 mL, 3.5 mL, 3.75 mL, 4 mL, 4.25 mL, 4.5 mL, 4.75 mL, 5 mL, 5.25 mL, 5.5 mL, 5.75 mL, or 6 mL). For example, the compositions described herein may be administered to a subject in a volume of about 10 mL (e.g., 9 mL, 10 mL, or 11 mL).

Any of the compositions described herein (e.g., a composition containing a plurality of UC-MSCs, a plurality of isolated exosomes, US-MSC secretome-conditioned cell culture medium, exosome-depleted UC-MSC-conditioned cell culture medium, or some combination thereof) may be administered to a subject having PTHS via a bolus (e.g., an IV bolus), a push (e.g., an IV push), or a drip (e.g., an IV drip). The compositions described herein may be administered to a subject over a period of 1 minute to 1 hour (e.g., 1 minute, 10 minutes, 20 minutes, 30 minutes, 40 minutes, 50 minutes, or 1 hour), 1 minute to 30 minutes (e.g., 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 21 minutes, 22 minutes, 23 minutes, 24 minutes, 25 minutes, 26 minutes, 27 minutes, 28 minutes, 29 minutes, or 30 minutes), or about 1 minute to about 10 minutes (e.g., 1 minute, 1.5 minutes, 2 minutes, 2.5 minutes, 3 minutes, 3.5 minutes, 4 minutes, 4.5 minutes, 5 minutes, 5.5 minutes, 6 minutes, 6.5 minutes, 7 minutes, 7.5 minutes, 8 minutes, 8.5 minutes, 9 minutes, 9.5 minutes, or 10 minutes).

The compositions described herein (e.g., a composition containing a plurality of UC-MSCs, a plurality of isolated exosomes, US-MSC secretome-conditioned cell culture medium, exosome-depleted UC-MSC-conditioned cell culture medium, or some combination thereof) may be administered to a subject having PTHS at a frequency of once every one, two, three, four, five, six, seven, eight, nine, ten, eleven, or twelve months. For example, the compositions described herein may be administered to a subject at a frequency of once every three months. In another example, the compositions described herein may be administered to a subject at a frequency of once every about three months (e.g., at a frequency of once every 81 days, 82 days, 83 days, 84 days, 85 days, 86 days, 87 days, 88 days, 89 days, 90 days, 91 days, 92 days, 93 days, 94 days, 95 days, 96 days, 97 days, 98 days, or 99 days). Administration of the compositions described herein may continue at a frequency described above for up to 1 year, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, or more. Administration of the compositions described herein may continue at a frequency described above for the life of the subject.

### B. Methods of Modifying TCF4 Expression in UC-MSCs

A further embodiment includes methods to increase TCF4 expression within the transplanted MSCs through methods including but not limited to epigenetic, enhancement of Wnt signaling, and genetic engineering of the UC-MSCs by transient or constitutive transfection, induced TCF4 expression and use of engineered secretory sequences to enhance TCF4 secretion from the UC-MSCs. An embodiment for increased TCF4 expression through epigenetic intervention is by use of an HDAC1 inhibitor(s) that is known to enhance function of UC-MSCs through increased migration, proliferation, and upregulation of Sirt-I, Oct¾, CXCR4, Hsp70, and TCF4 transcription factor as well (e.g., see U.S. application Ser. No. 17/239,513 and Hennig, K M, et al, Mol Neuropsychiatry 2017; 3: 53-71, each of which is incorporated by reference). In an embodiment, HDAC1 inhibition occurs by exposure of cultured UC-MSCs to 500 nM curcumin in the culture medium for two weeks or more prior to harvesting of the cells for treatment of a PTHS patient in need.

Enhancement of Wnt signaling may occur by methods well known to those skilled in the art, such as through activation of Wnt receptors, frizzled by Wnt agonists, and by exposure of cultured UC-MSCs to 5 to 10% Wnt-3a conditioned medium for two weeks or more prior to harvesting of the cells for treatment of a PTHS patient in need (Hennig, K M, et al, Mol Neuropsychiatry 2017; 3: 53-71). Measurement of TCF-4 expression in UC-MSCs can be performed by, e.g., use of high sensitivity qRT-PCR. Enhancement of TCF-4 expression in UC-MSCs may occur by either curcumin or Wnt-3a treatment alone or in combination.

An optional embodiment includes use of genetic engineering technology to enhance TCF4 expression in UC-MSCs prior to transplantation into a PTHS patient in need. Expression vectors would include appropriate promoters, signal sequences to direct TCF4 secretion/uptake from UC-MSCs, use of qRT-PCR monitoring of expression and other standard procedures well-known to those skilled in the art.

The methods of treatment described herein may include administering to a subject an effective amount of a composition containing a plurality of UC-MSCs expressing TCF4 (e.g., TCF4 mRNA and/or protein). As discussed above, the expression of TCF4 in these UC-MSCs may be modified (e.g., increased or prolonged, relative to a control (e.g., untreated cells) by any of several methods described herein. For example, modifying TCF4 expression in UC-MSCs can involve genetically manipulating the UC-MSCs to express exogenous (fully functional) TCF4 and/or chemically inducing (e.g., in vitro or in vivo) TCF4 gene expression (e.g., by epigenetic manipulation or signal transduction mechanisms).

i. Genetic Manipulation of TCF4 Expression in UC-MSCs

The methods of treating a subject having PTHS described herein may a plurality of isolated UC-MSCs that express TCF4 (e.g., mRNA and/or protein) or a TCF4 fragment or isoform thereof. Since a PTHS subject may lack TCF4 or may have a mutant and/or non-functional TCF4, expressing a non-mutant/functional TCF4 in UC-MSCs is crucial for the methods described herein. Therefore, genetic manipulation of the UC-MSCs may be employed.

UC-MSCs may be isolated from the subject having PTHS or isolated from a healthy donor (e.g., a subject that does not have PTHS). UC-MSCs isolated from a healthy donor may be used in the methods of treating PTHS described herein. UC-MSCs may be modified (e.g., modified UC-MSCs) as described herein. For example, UC-MSCs that do not express TCF4, express a mutant of TCF4, or express a non-functional TCF4 (e.g., UC-MSCs from a subject having PTHS) may be modified to express one, two, three, four, five, or more copies of a non-mutant and/or functional TCF4 (e.g., TCF4 mRNA and/or protein). In another example, UC-MSCs that express TCF4 (e.g., UC-MSCs from a healthy donor) may be modified to augment their endogenous TCF4 expression. Modified UC-MSCs have an increased expression of TCF4 mRNA and/or protein levels relative to unmodified UC-MSCs.

UC-MSCs may be modified by introducing exogenous genetic material (e.g., DNA, cDNA, RNA, or mRNA) into the cell. For example, a nucleic acid vector, plasmid, circular RNA, or mRNA molecule may be introduced into the UC-MSCs, e.g., by way of transfection (e.g., microinjection, optical transfection, biolistic transfection, electroporation, nucleofection, lipofection, sonoporation, and magnetofection). The exogenous genetic material (e.g., nucleic acid vector, plasmid, circular RNA, or mRNA molecule) may contain one or more (e.g., 1, 2, 3, 4, 5, 6, 7, 8, 9, 10 or more) nucleotide sequences encoding one or more copies of TCF4 (e.g., TCF4 mRNA or protein). Such exogenous expression of TCF-4 in the UC-MSCs (e.g., UC-MSCs from a healthy donor or a subject having PTHS) will augment endogenous TCF4 expression in the cell and promote the downstream signaling effects of TCF4. The exogenous genetic material (e.g., nucleic acid vector, plasmid, circular RNA, or mRNA molecule) may contain one or more (e.g., 1, 2, 3, 4, 5, 6, 7, 8, 9, 10 or more) nucleotide sequence encoding a Wnt signaling agonist. Such exogenous expression of one or more a Wnt signaling agonist in the UC-MSCs (e.g., UC-MSCs from a healthy donor) may promote endogenous TCF4 expression in the cell, thereby promoting the downstream signaling effects of TCF4. The exogenous genetic material (e.g., nucleic acid vector, plasmid, circular RNA, or mRNA molecule) may contain one or more (e.g., 1, 2, 3, 4, 5, 6, 7, 8, 9, 10 or more) nucleotide sequences encoding a frizzled-signaling agonist (e.g., a Wnt 3a sequence described in Sukarawan et al., Int Endod J., 56(4):514-529 (2023), herein incorporated by reference; or provided under NCBI Reference Sequence: NM_033131.4). Such exogenous expression of one or more frizzled-signaling agonist in the UC-MSCs (e.g., UC-MSCs from a healthy donor) may promote endogenous TCF4 expression in the cell, thereby promoting the downstream signaling effects of TCF4. The exogenous genetic material (e.g., nucleic acid vector, plasmid, circular RNA, or mRNA molecule) may be one or more nucleic acid molecules containing one or more nucleotide sequences (e.g., DNA, cDNA, RNA, or mRNA sequences) encoding any one or more of the following: TCF4, a Wnt signaling agonist, and a frizzled-signaling agonist (e.g., Wnt agonist-1 or Wnt 3a).

Exemplary TCF4 genes, mRNA transcript, and protein sequences to be expressed in UC-MSCs are provided in Table 1 below.

TABLE 1
Exemplary TCF4 Sequences of Functional Human TCF4

| NCBI Accession Numbers | | | Sequence Type |
|---|---|---|---|
| NC_000018.10 | | | Genomic sequence |
| NM_001243226.3 | NM_001369579.1 | NM_001348212.2 | Human TCF4 mRNA |
| NM_001369584.1 | NM_001369583.1 | NM_001348213.2 | transcript sequences |
| NM_001369576.1 | NM_001369575.1 | NM_001243233.2 | |
| NM_001369577.1 | NM_001369573.1 | NM_001330605.3 | |
| NM_001369582.1 | NM_001369570.1 | NM_001348214.2 | |
| NM_001243227.2 | NM_001369574.1 | NM_001243235.2 | |
| NM_001369586.1 | NM_001330604.3 | NM_001243234.2 | |
| NM_001369571.1 | NM_003199.3 | NM_001348215.2 | |
| NM_001369567.1 | NM_001083962.2 | NM_001243236.2 | |
| NM_001243228.2 | NM_001369581.1 | NM_001348216.2 | |
| NM_001243230.2 | NM_001369580.1 | NM_001348220.1 | |
| NM_001369569.1 | NM_001348219.2 | NM_001306207.1 | |
| NM_001369572.1 | NM_001348218.2 | NM_001348217.1 | |
| NM_001369568.1 | NM_001243231.2 | NM_001306208.1 | |
| NM_001369585.1 | NM_001348211.2 | NM_001243232.1 | |
| NM_001369578.1 | | | |
| NP_001230155.2 | NP_001356508.1 | NP_001335141.1 | Human TCF4 protein |
| NP_001356513.1 | NP_001356512.1 | NP_001335142.1 | sequences |
| NP_001356505.1 | NP_001356504.1 | NP_001230162.1 | |
| NP_001356506.1 | NP_001356502.1 | NP_001317534.1 | |
| NP_001356511.1 | NP_001356499.1 | NP_001335143.1 | |
| NP_001230156.1 | NP_001356503.1 | NP_001230164.1 | |
| NP_001356515.1 | NP_001317533.1 | NP_001230163.1 | |
| NP_001356500.1 | NP_003190.1 | NP_001335144.1 | |
| NP_001356496.1 | NP_001077431.1 | NP_001230165.1 | |
| NP_001230157.1 | NP_001356510.1 | NP_001335145.1 | |
| NP_001230159.1 | NP_001356509.1 | NP_001335149.1 | |
| NP_001356498.1 | NP_001335148.1 | NP_001293136.1 | |
| NP_001356501.1 | NP_001335147.1 | NP_001335146.1 | |
| NP_001356497.1 | NP_001230160.1 | NP_001293137.1 | |
| NP_001356514.1 | NP_001335140.1 | NP_001230161.1 | |
| NP_001356507.1 | | | |

ii. In Vitro Chemical Induction of TCF4 Expression in UC-MSCs

Prior to the administration of the composition, UC-MSCs (including modified UC-MSCs) may be pre-treated with one or more Wnt pathway activators. Wnt pathway activators may stimulate the expression of endogenous TCF4 in the UC-MSC, thereby increasing endogenous TCF4 expression relative to an untreated UC-MSC. Wnt pathway activators include, but are not limited to, a histone deacetylase 1 (HDAC1) inhibitor (e.g., vorinostat, romidepsin, belinostat, panobinostat, valproic acid, entinostat, curcumin, quercetin, and RG2833), a Wnt-signaling agonist (e.g., L-Quebrachital), a frizzled-signaling agonist (e.g., Wnt agonist-1 or Wnt-3a), and a glycogen synthase kinase-3p (GSK-3B3) inhibitor (e.g., indirubin-3′-oxime, landuviglusib (CHIR-99821), and KY19382).

By way of example, the UC-MSC may be pre-treated by contacting a plurality of UC-MSCs with about 1 nanomolar (nM) to about 10 micromolar (pM) (e.g., 1 nM, 50 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, or 10 μM) about 100 nM to about 1 μM (e.g., 100 nM, 150 nM, 200 nM, 250 nM, 300 nM, 350 nM, 400 nM, 450 nM, 500 nM, 550 nM, 600 nM, 650 nM, 700 nM, 750 nM, 800 nM, 850 nM, 900 nM, 950 nM, or 1 μM), about 400 nM to about 600 nM (e.g., 400 nM, 425 nM, 450 nM, 475 nM, 500 nM, 525 nM, 550 nM, 575 nM, or 600 nM), or about 500 nM (e.g., 450 nM, 460 nM, 470 nM, 480 nM, 490 nM, 500 nM, 510 nM, 520 nM, 530 nM, 540 nM, or 550 nM) of an HDAC1 inhibitor (e.g., vorinostat, romidepsin, belinostat, panobinostat, valproic acid, entinostat, curcumin, quercetin, and RG2833). In yet another example, the plurality of UC-MSCs may be contacted with about 500 nM of curcumin.

In another example, the UC-MSC may be pre-treated by contacting a plurality of UC-MSCs with a frizzled-signaling agonist (e.g., Wnt agonist-1 or Wnt-3a). The frizzled-signaling agonist (e.g., Wnt agonist-1 or Wnt-3a) may be provided in a final concentration of about 5 to about 20 ng/mL (e.g., 5 ng/mL, 6 ng/mL, 7 ng/mL, 8 ng/mL, 9 ng/mL, 10 ng/mL, 11 ng/mL, 12 ng/mL, 13 ng/mL, 14 ng/mL, 15 mg/mL, 16 ng/mL, 17 ng/mL, 18 ng/mL, 19 ng/mL, or 20 ng/mL). Alternatively, the frizzled-signaling agonist (e.g., Wnt agonist-1 or Wnt-3a) may be provided in a conditioned cell culture medium at a concentration of 5-15% w/v (e.g., 5%, 6%, 7%, 8%, 9%, 10%, 11%, 12%, 13%, 14%, or 15% w/v). The conditioned cell culture medium may be Wnt-3a-conditioned cell culture medium or Wnt agonist-1-conditioned cell culture medium. The conditioned cell culture medium may be derived from a cell culture containing a mouse L-cell line (e.g., American Type Culture Collection (ATCC) catalog: CRL-2648). In particular, the plurality of UC-MSCs is contacted with about 10% (e.g., 9%, 9.5%, 10%, 10.5%, or 11%) Wnt-3a-conditioned cell culture medium. Such cell culture medium is described in Zhao et al., J Biomol Screen., 17(9):1252-63 (2012) and Willert et al., Nature, 423(6938):448-52 (2003), each of which is incorporated herein by reference.

Prior to the administration of the composition, pre-treatment of the UC-MSCs may include contacting the UC-MSCs with one, two, three, four, five, or more of the Wnt pathway activators described above (e.g., vorinostat, romidepsin, belinostat, panobinostat, valproic acid, entinostat, curcumin, quercetin, RG2833, L-Quebrachital, Wnt agonist-1, Wnt-3a, indirubin-3′-oxime, landuviglusib (CHIR-99821), KY19382). In doing so, TCF4 expression in the UC-MSCs may increase relative to an untreated UC-MSC, which may bolster or augment the PTHS treatments described herein. In particular, the plurality of UC-MSCs can be contacted with an HDAC1 inhibitor (e.g., curcumin and/or quercetin) and a Wnt-3a protein (e.g., in isolated form or present in 10% Wnt-3a-conditioned cell culture medium).

Contacting the UC-MSCs with the Wnt pathway activators described above (e.g., the HDAC1 inhibitors, Wnt-signaling agonists, frizzled-signaling agonists, and/or GSK inhibitors) may occur for about 1 day to about 6 weeks (e.g., 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, or 42 days), about 1-3 weeks (e.g., 1 week, 10 days, 11 days, 2 weeks, 17 days, 18 days, 3 weeks), or about 2 weeks (e.g., 13 days, 14 days, or 15 days).

iii. In Vivo Chemical Induction of TCF4 Expression in UC-MSCs

In another embodiment, the patient is pretreated with a combination of curcumin and quercetin prior to administration of the umbilical cord-derived hMSCs to the patient. In this embodiment, the patient is treated until their serum concentration of each compound is approximately 500 nM. Another treatment embodiment involves pre-treatment of MSCs to increase expression of the TCF4 gene in the MSCs prior to or after transplantation into PTHS patients. This pre-treatment involves preferably inhibition of histone deacetylase 1 (HDAC1) by various agents including curcumin. Another treatment that enhances TCF4 expression includes activation of the Wnt/beta catenin signaling pathway including use of Wnt receptor ligands or agonists.

Prior to the administration of the composition, the subject may be treated (e.g., pre-treated) with one or more Wnt pathway activators described herein (e.g., a HDAC1 inhibitor, Wnt-signaling agonist, frizzled-signaling agonist, or GSK-3p inhibitor). Concurrently with the administration of the composition, the subject may be treated (e.g., co-treated) with one or more Wnt pathway activators described herein (e.g., a HDAC1 inhibitor, Wnt-signaling agonist, frizzled-signaling agonist, or GSK inhibitor). Following the administration of the composition, the subject may be treated (e.g., post-treated) with one or more Wnt pathway activators described herein (e.g., a HDAC1 inhibitor, Wnt-signaling agonist, frizzled-signaling agonist, or GSK inhibitor). Thus, treatment with the one or more Wnt patway activators described herein is envisioned to occur prior to, concurrently with, and/or following the administration of a composition containing a plurality of UC-MSCs.

Prior to, concurrently with, and/or following the administration of a composition containing a plurality of UC-MSCs, the subject (e.g., a subject with PTHS) may be treated with one or more Wnt pathway activators. Increasing the concentration of a Wnt pathway activator in the subject's serum can facilitate the UC-MSCs contact with the Wnt pathway activator, thereby stimulating the expression of endogenous TCF4 in the UC-MSC. Wnt pathway activators include, but are not limited to, a histone deacetylase 1 (HDAC1) inhibitor (e.g., vorinostat, romidepsin, belinostat, panobinostat, valproic acid, entinostat, curcumin, quercetin, and RG2833), a Wnt-signaling agonist (e.g., L-Quebrachital), a frizzled-signaling agonist (e.g., Wnt agonist-1 or Wnt-3a), and a glycogen synthase kinase-3p (GSK-3p) inhibitor (e.g., indirubin-3′-oxime, landuviglusib (CHIR-99821), and KY19382).

Prior to, concurrently with, and/or following the administration of a composition containing a plurality of UC-MSCs, the subject may be administered an amount of the HDAC1 inhibitor (e.g., vorinostat, romidepsin, belinostat, panobinostat, valproic acid, entinostat, curcumin, quercetin, and RG2833) sufficient to achieve a serum concentration about 0.1 nM to about 1000 nM (e.g., about 0.1 nM to 10 nM, about 1 nM to about 100 nM, about 10 nM to about 1000 nM, about 200 nM to about 500 nM, about 350 nM to about 700 nM, or about 500 nM to about 1000 nM, e.g., 01 nM, 0.5 nM, 1 nM, 5 nM, 10 nM, 20 nM, 30 nM, 40 nM, 50 nM, 60 nM, 70 nM, 80 nM, 90 nM, 100 nM, 125 nM, 150 nM, 175 nM, 200 nM, 225 nM, 250 nM, 275 nM, 300 nM, 325 nM, 350 nM, 375 nM, 400 nM, 425 nM, 450 nM, 475 nM, 500 nM, 525 nM, 550 nM, 575 nM, 600 nM, 625 nM, 650 nM, 675 nM, 700 nM, 725 nM, 750 nM, 775 nM, 800 nM, 825 nM, 850 nM, 875 nM, 900 nM, 925 nM, 950 nM, 975 nM, or 1000 nM). In particular, the subject is administered an amount of an HDAC1 inhibitor (e.g., curcumin) sufficient to achieve a serum concentration of about 500 nM (e.g., 450 nM, 460 nM, 470 nM, 480 nM, 490 nM, 500 nM, 510 nM, 520 nM, 530 nM, 540 nM, or 550 nM). For example, the subject is administered an amount of an HDAC1 inhibitor (e.g., curcumin) sufficient to achieve a serum concentration of 500 nM.

Curcumin can act as a pleiotropic agent since it appears to act as both a HDAC1 inhibitor and a GSK-3 p inhibitor; for example, curcumin can alter the expression level of targets of both HDAC1 inhibitors and GSK-3p inhibitors (e.g., Sirt-1, CXCR4, HSP70, Oct ¾, and FGF21). In some embodiments, expression of Sirt-1 is increased by 200-to-300-fold, expression of CXCR4 is increased by 10-to-20-fold, heat shock protein 70 levels are increased by up to 20-fold, Oct ¾ is increased by approximately 10-to-20-fold, within the patient receiving the UC-MSCs. In other embodiments, expression of FGF21 can be increased by 10-to-20-fold, and activation of CXCR4 and MMP9 can be increased by administering substances that induce GSK-3p inhibition and HDAC1 inhibition.

The HDAC1 inhibitor vorinostat (e.g., ZOLINZA®) may also be administered in an amount of about 400 mg (e.g., 360 mg, 370 mg, 380 mg, 390 mg, 400 mg, 410 mg, 420 mg, 430 mg, or 440 mg) and/or in an amount sufficient to achieve geometric mean values of a maximum plasma concentration (Cₘₐₓ) and an area under the plasma concentration versus time curve (AUC₀₋ᵢₙₜ) of 1.2±0.53 μM and 6.0±2.0 μM*hr, respectively. Vorinostat may be administered orally as a capsule or tablet.

The HADC1 inhibitor romidepsin (e.g., ISTODAX®) may also be administered in an amount of about 14 mg/m² (e.g., 12.6 mg/m², 13 mg/m², 13.5 mg/m², 14 mg/m², 14.5 mg/m², 15 mg/m², or 15.4 mg/m²) over a 4-hour period, such as on days 1, 8, and 15 of a 28-day cycle and/or in an amount sufficient to achieve geometric mean values of a maximum plasma concentration (Cₘₐₓ) and an area under the plasma concentration versus time curve (AUC₀₋ᵢₙₜ) of about 377 ng/mL (e.g., 340 ng/mL, 360 ng/mL, 380 ng/mL, 400 ng/mL, or 414 ng/mL) and about 1549 ng*hr/mL (e.g., 1395 ng*hr/mL, 1440 ng*hr/mL, 1480 ng*hr/mL, 1520 ng*hr/mL, 1560 ng*hr/mL, 1600 ng*hr/mL, 1640 ng*hr/mL, 1680 ng*hr/mL, or 1703 ng*hr/mL) respectively. Romidepsin may be administered by IV.

The HDAC1 inhibitor belinostat (e.g., BELEODAQ®) may also be administered in an amount of about 1,000 mg/m² (e.g., 900 mg/m², 950 mg/m², 1,000 mg/m², 1,050 mg/m², 1,100 mg/m²) over a 30 minute period, such as on days 1-5 of a 21-day cycle. Belinostat may be administered by IV (e.g., intravenous infusion).

The HDAC1 inhibitor panobinostat (FARYDAK®) may also be administered in an amount of about 20 mg (e.g., 18 mg, 19 mg, 20 mg, 21 mg, or 22 mg) every other day, such as on days 1, 3, 5, 8, 10, and 12 of a 21-day cycle. Panobinostat may be administered orally as a capsule or tablet.

The HDAC1 inhibitor valproic acid (e.g., DEPAKENE®) may also be administered in an amount of about 10 to 60 mg/kg/day (e.g., 10 to 40 mg/kg/day, 30 to 50 mg/kg/day or 40 to 60 mg/kg/day, e.g., 10 mg/kg/day, 15 mg/kg/day, 20 mg/kg/day, 25 mg/kg/day, 30 mg/kg/day, 35 mg/kg/day, 40 mg/kg/day, 45 mg/kg/day, 50 mg/kg/day, 55 mg/kg/day, or 60 mg/kg/day). Valproic acid may be administered orally as a capsule or tablet.

The HDAC1 inhibitor entinostat may also be administered in an amount of about 2 mg/m² to about 12 mg/m² (e.g., 2 mg/m² to 9 mg/m² or 6 mg/m² to 12 mg/m², e.g., 1 mg/m², 2 mg/m², 3 mg/m², 4 mg/m², 5 mg/m², 6 mg/m², 7 mg/m², 8 mg/m², 9 mg/m², or 10 mg/m², 11 mg/m², or 12 mg/m²). Entinostat may be administered orally as a capsule or tablet.

The HDAC1 inhibitor curcumin may also be administered in an amount of about 1 g to about 8 g per day (e.g., 1 g to 4 g per day, 2 g to 6 g per day, or 4 g to 8 g per day, e.g., 1 g per day, 2 g per day, 3 g per day, 4 g per day, 5 g per day, 6 g per day, 7 g per day, or 8 g per day).

The HDAC1 inhibitor quercetin may also be administered in an amount of about 250 mg to about 5000 mg per day (e.g., 250 mg to 1000 mg per day, 500 mg to 2000 mg per day, 1500 mg to 3000 mg per day, 2000 mg to 4000 mg per day, or 3000 mg to 5000 mg per day, e.g., 250 mg per day, 300 mg per day, 400 mg per day, 500 mg per day, 600 mg per day, 700 mg per day, 800 mg per day, 900 mg per day, 1000 mg per day, 1100 mg per day, 1200 mg per day, 1300 mg per day, 1400 mg per day, 1500 mg per day, 1600 mg per day, 1700 mg per day, 1800 mg per day, 1900 mg per day, 2000 mg per day, 2100 mg per day, 2200 mg per day, 2300 mg per day, 2400 mg per day, 2500 mg per day, 2600 mg per day, 2700 mg per day, 2800 mg per day, 2900 mg per day, 3000 mg per day, 3100 mg per day, 3200 mg per day, 3300 mg per day, 3400 mg per day, 3500 mg per day, 3600 mg per day, 3700 mg per day, 3800 mg per day, 3900 mg per day, 4000 mg per day, 4100 mg per day, 4200 mg per day, 4300 mg per day, 4400 mg per day, 4500 mg per day, 4600 mg per day, 4700 mg per day, 4800 mg per day, 4900 mg per day, or 5000 mg per day), The HDAC1 inhibitor RG2833 may also be administered in an amount of about 30 mg to about 240 mg per day (e.g., 30 mg to 100 mg per day, 60 mg to 150 mg per day, 100 mg to 200 mg per day, or 150 mg to 240 mg per day, e.g, 30 mg per day, 40 mg per day, 50 mg per day, 60 mg per day, 70 mg per day, 80 mg per day, 90 mg per day, 100 mg per day, 110 mg per day, 120 mg per day, 130 mg per day, 140 mg per day, 150 mg per day, 160 mg per day, 170 mg per day, 180 mg per day, 190 mg per day, 200 mg per day, 210 mg per day, 220 mg per day, 230 mg per day, or 240 mg per day).

Prior to, concurrently with, and/or following the administration of a composition containing a plurality of UC-MSCs, the subject may be administered an amount of the GSK-3p inhibitor (e.g., indirubin-3′-oxime) sufficient to achieve a serum concentration about 20 to about 500 nM (e.g., 16 nM, 20 nM, 30 nM, 40 nM, 50 nM, 60 nM, 70 nM, 80 nM, 90 nM, 100 nM, 125 nM, 150 nM, 175 nM, 200 nM, 225 nM, 250 nM, 275 nM, 300 nM, 325 nM, 350 nM, 375 nM, 400 nM, 425 nM, 450 nM, 475 nM, 500 nM, 525 nM, or 550 nM). In particular, the subject is administered an amount of an GSK-3p inhibitor (e.g., indirubin-3′-oxime) sufficient to achieve a serum concentration of about 200 nM (e.g., 160 nM, 170 nM, 180 nM, 190 nM, 200 nM, 210 nM, or 220 nM). For example, the subject is administered an amount of a GSK-3p inhibitor (e.g., indirubin-3′-oxime) sufficient to achieve a serum concentration of 500 nM.

Prior to, concurrently with, and/or following the administration of a composition containing a plurality of UC-MSCs, the subject may be administered an amount of the Wnt-signaling agonist (e.g., L-Quebrachital), a frizzled-signaling agonist (e.g., Wnt agonist-1 or Wnt-3a), or a glycogen synthase kinase-3p (GSK-3p) inhibitor (e.g., indirubin-3′-oxime, landuviglusib (CHIR-99821), and KY19382). In particular, the subject is administered an amount of a frizzled-signaling agonist (e.g., Wnt-3a) sufficient to achieve a serum concentration of about 1 ng/mL to about 100 ng/mL (e.g., 9.9 ng/mL, 1 ng/mL, 10 ng/mL, 20 ng/mL, 30 ng/mL, 40 ng/mL, 50 ng/mL, 60 ng/mL, 70 ng/mL, 80 ng/mL, 90 ng/mL, 100 ng/mL, or 110 ng/mL), about 2 ng/mL to about 50 ng/mL (e.g., 1.8 ng/mL, 2 ng/mL, 4 ng/mL, 8 ng/mL, 16 ng/mL, 20 ng/mL, 24 ng/mL, 28 ng/mL, 32 ng/mL, 36 ng/mL, 40 ng/mL, 44 ng/mL, 48 ng/mL, 50 ng/mL, or 55 ng/mL), or about 5 ng/mL to about 20 ng/mL (e.g., 4.5 ng/mL, 5 ng/mL, 6 ng/mL, 7 ng/mL, 8 ng/mL, 9 ng/mL, 10 ng/mL, 11 ng/mL, 12 ng/mL, 13 ng/mL, 14 ng/mL, 15 ng/mL, 16 ng/mL, 17 ng/mL, 18 ng/mL, 19 ng/mL, 20 ng/mL, or 22 ng/mL). For example, the subject is administered an amount of a frizzled-signaling agonist (e.g., Wnt-3a) sufficient to achieve a serum concentration of 5 ng/mL to 20 ng/mL.

Prior to, concurrently with, and/or following the administration of a composition containing a plurality of UC-MSCs, the subject may be administered one, two, three, four, five, or more of the Wnt pathway activators described above (e.g., vorinostat, romidepsin, belinostat, panobinostat, valproic acid, entinostat, curcumin, quercetin, RG2833, L-Quebrachital, Wnt agonist-1, Wnt-3a, indirubin-3′-oxime, landuviglusib (CHIR-99821), KY19382). For example, the subject may be administered an amount of an HDAC1 inhibitor (e.g., curcumin) sufficient to achieve a serum concentration of 500 nM and an amount of a frizzled-signaling agonist (e.g., Wnt-3a) sufficient to achieve a serum concentration of 5 ng/mL, 6 ng/mL, 7 ng/mL, 8 ng/mL, 9 ng/mL, 10 ng/mL, 11 ng/mL, 12 ng/mL, 13 ng/mL, 14 ng/mL, 15 ng/mL, 16 ng/mL, 17 ng/mL, 18 ng/mL, 19 ng/mL, or 20 ng/mL in the subject having PTHS. Increasing the concentration of more than one Wnt pathway activator in the subject's serum can better facilitate the UC-MSCs contact with the Wnt pathway activator, thereby stimulating the expression of endogenous TCF4 in the UC-MSC. In a further example, the subject may also be administered a third, fourth, or fifth Wnt pathway activator selected from vorinostat, romidepsin, belinostat, panobinostat, valproic acid, entinostat, quercetin, RG2833, L-Quebrachital, Wnt agonist-1, indirubin-3′-oxime, landuviglusib (CHIR-99821), KY19382.

Any of Wnt pathway activator described herein (e.g., a HDAC1 inhibitor, Wnt-signaling agonist, frizzled-signaling agonist, or GSK inhibitor) may be administered to a subject having PTHS in a volume of about 0.05 milliliters (mL) to about 15 mL (e.g., 0.05 mL, 0.1 mL, 0.2 mL, 0.3 mL, 0.4 mL, 0.5 mL, 0.6 mL, 0.7 mL, 0.8 mL, 0.9 mL, 1 mL, 3 mL, 5 mL, 7 mL, 10 mL, 11 mL, 12 mL, 13 mL, 14 mL, or 15 mL), about 1 mL to about 10 mL (e.g., 1 mL, 2 mL, 3 mL, 4 mL, 5 mL, 6 mL, 7 mL, 8 mL, 9 mL, or 10 mL), or about 1 mL to about 6 mL, (e.g., 1 mL, 1.25 mL, 1.5 mL, 1.75 mL, 2 mL, 2.25 mL, 2.5 mL, 2.75 mL, 3 mL, 3.25 mL, 3.5 mL, 3.75 mL, 4 mL, 4.25 mL, 4.5 mL, 4.75 mL, 5 mL, 5.25 mL, 5.5 mL, 5.75 mL, or 6 mL).

Any of Wnt pathway activator described herein (e.g., a HDAC1 inhibitor, Wnt-signaling agonist, frizzled-signaling agonist, or GSK inhibitor) may be administered to a subject having PTHS via a bolus (e.g., an IV bolus), a push (e.g., an IV push), or a drip (e.g., an IV drip). Further, administration of the Wnt pathaway activator may be over a period of 1 minute to 1 hours (e.g., 1 minute, 10 minutes, 20 minutes, 30 minutes, 40 minutes, 50 minutes, or 1 hour), 1 minute to 30 minutes (e.g., 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 21 minutes, 22 minutes, 23 minutes, 24 minutes, 25 minutes, 26 minutes, 27 minutes, 28 minutes, 29 minutes, or 30 minutes), or about 1 minute to about 10 minutes (e.g., 1 minute, 1.5 minutes, 2 minutes, 2.5 minutes, 3 minutes, 3.5 minutes, 4 minutes, 4.5 minutes, 5 minutes, 5.5 minutes, 6 minutes, 6.5 minutes, 7 minutes, 7.5 minutes, 8 minutes, 8.5 minutes, 9 minutes, 9.5 minutes, or 10 minutes).

Administration of the Wnt pathway activator may occur prior to, concurrently with, and/or following the administration of a composition described herein.

### C. PTHS Subject in Need of Treatment

The methods of treatment described herein are intended to treat a subject with PTHS. A subject with PTHS may exhibit an impairment in motor function, communication, sleep, gastrointestinal health, breathing, cognition, and/or adaptive behavior. A subject with PTHS may have a mutation (e.g., insertion, deletion, or substitution) or defect in the TCF4 gene that reduces or abrogates TCF4 (e.g., mRNA or protein) expression and/or TCF4's function as a transcription factor. For example, a subject may have any pathogenic or likely pathogenic TCF4 described herein (e.g., see Table 1 in Mary et al., Eur. J. Human Genetics, 26(7):996-1006 (2018)).

A PTHS subject may be a neonate (e.g., less than 29 days of age), an infant (e.g., less than one year of age), a child (e.g., between one year and 10 years of age), an adolescent (e.g., between 10 years and 19 years of age) or an adult (e.g., over 19 years of age).

Any PTHS subject (e.g., neonates, infants, children, or adults) may be administered a composition described herein (e.g., a composition containing a plurality of UC-MSCs, a plurality of isolated exosomes, US-MSC secretome-conditioned cell culture medium, exosome-depleted UC-MSC-conditioned cell culture medium, or some combination thereof) using the methods described herein. For example, the subject (e.g., neonate, infant, child, or adult) may be administered the composition by IV infusion at 1×10⁶ to 2.5×10⁶ cells/kg of body weight. Further, administration may occur 3 to 4 times per year. The number of administrations per year can depend on need, as determined can a clinician.

A subject may be treated for PTHS following the methods described herein after their diagnosis of PTHS (e.g., within 1 hour, within 1 day, within 1 week, within 1 month, within 6 months, or within 1 year from the subject's diagnosis).

i. Therapeutic Endpoints

The patient being treated may be examined for the following endpoints before and/or after treatment:
Motor function: Vineland Motor Subscale-3 Caregiver Interview, Bayley Scales of Infant Development (BSID-4), Video capture of gait in coronal and sagittal plane, WeeFIM, magneto-inertial monitoring
Communication: WeeFIM, Observer-Reported Communication Ability Measure (ORCA)
Sleep: Sleep diary
Gastrointestinal: Gastrointestinal Health Questionnaire
Breathing disorders: Caregiver diary
Cognition: BSID-4
Adaptive behavior: WeeFIM, Q-global Vineland assessment, Vineland behavioral scalers, Aberrant Behavior Checklist-2
Parent assessment
Pediatric Quality of Life
Laboratory assessment
TCF4 expression by qRT-PCR
Comprehensive metabolic panel
Complete Blood Chemistry
Coagulation (PT, PTT and D-dimer)
Inflammation markers (CRP, ESR, IL-6, C3, C4, LDH, ferritin)
Th17/Treg ratio
Global Clinical Assessment:
   PTHS-specific Clinical Global Impression Scale—Overall improvement
   PTHS-specific Clinical Global Impression Scale (CGI-S)—overall and domain

A subject with PTHS may exhibit a reduced TCF4 (e.g., mRNA and/or protein) expression level in excitatory neurons, inhibitory neurons, astrocytes, oligodendrocytes and/or lymphocytes relative to a healthy subject. This may be caused by a monoallelic mutation or deletion in TCF4 that reduces TCF4 expression relative to a healthy subject without the mutation or deletion in TCF4. The methods of treatment described herein may increase cellular TCF4 expression in the subject relative to the TCF4 expression level prior to administration of the composition (e.g., the composition containing a plurality of UC-MSCs, a plurality of isolated exosomes, US-MSC secretome-conditioned cell culture medium, exosome-depleted UC-MSC-conditioned cell culture medium, or some combination thereof) to the subject.

Any suitable laboratory technique for determining RNA (e.g., mRNA) expression levels of TCF4 in can be used, including, but not limited to, PCR, RT-PCR, qPCR, RT-qPCR, microarray analysis, Northern blot, MASSARRAY® technique, SAGE, and RNA-sequencing. Further, any suitable laboratory technique for determining expression levels of TCF4 can be used, including, but not limited, to flow cytometry (FC), fluorescence-activated cell sorting (FACS) Western blot, enzyme-linked immunosorbent assay (ELISA), mass spectrometry (MS), immunofluorescence (IF), immunoprecipitation (IP), radioimmunoassay, dot blotting, high performance liquid chromatography (HPLC), surface plasmon resonance, optical spectroscopy, and immunohistochemistry (IHC). Any increase (e.g., a 5%, 10% 30%, 40%, 50%, 60%, 70% 80%, 90%, 100%, or more increase) in the expression level of TCF4 is indicative of treatment efficacy.

A subject with PTHS may also exhibit an impairment in motor function, communication, sleep, gastrointestinal health, breathing, cognition, and/or adaptive behavior. Thus, a clinical assessment of one or more of these features may be used to assess the progression of PTHS in the subject and/or assess the efficacy of the methods of treatment described herein. While any standard clinical assessment for PTHS is envisioned, several are exemplified below.

Motor function impairment may be assessed by one or more of a Vineland Motor Subscale-3 caregiver interview, a Bayley Scales of Infant Development (BSID-4) questionnaire, a video capture of gait in coronal and sagittal plane, a Functional Independence Measure for Children (WeeFIM), and an Observer-Reported Communication Ability Measure (ORCA). Communication impairment may be assessed by WeeFIM and/or ORCA. Sleep impairment may be assessed by a sleep diary questionnaire. Gastrointestinal impairment may be assessed by a gastrointestinal health questionnaire. Breathing impairment may be assessed by spirometry or a caregiver diary. Cognition impairment may be assessed by a BSID-4 questionnaire. Lastly, adaptive behavior impairment may be assessed by one or more of a Q-global Vineland assessment, a Vineland behavioral scalers questionnaire, or an Aberrant Behavior Checklist-2.

The methods of treatment described herein may improve, mitigate, slow down, or halt the progression of one or more of these impairments, relative to an untreated control, as determined by one or more clinical assessments described above.

ii. TCF4 Mutations

A subject with PTHS may be identified by genotyping their TCF4 gene and identifying a pathogenic mutation. Any standard genotyping technique may be used, such as PCR, RT-PCR, qPCR, RT-qPCR, microarray analysis, Northern blot, MASSARRAY® technique, SAGE, RNA-sequencing.

Exemplary pathogenic PTHS-associated TCF4 mutations are: NM_001083962.2 (TCF4): c.1817_1828del (p.Thr606_Leu609del); NM_001083962.2 (TCF4): c.145+1 G>A; NM_001083962.2 (TCF4): c.550-2A>G; NM_001083962.2 (TCF4): c.2010_2011 del (p.Gln670fs); NM_001083962.2 (TCF4): c.1916_1917del (p.Arg639fs); NM_001083962.2 (TCF4): c.1086G>A (p.Trp362Ter); NM_001083962.2 (TCF4): c.1485_1486dup (p.Gly496fs); NM_001083962.2 (TCF4): c.1469C>G (p.Pro490Arg); NM_001083962.2 (TCF4): c.1147-2A>G; NM_001083962.2 (TCF4): c.790-1G>A; NM_001083962.2 (TCF4): c.1772T>C (p.Leu591 Pro); NM_001083962.2 (TCF4): c.550-1G>A; NM_001083962.2 (TCF4): c.550-12_550-2del; NM_001083962.2 (TCF4): c.1619A>G (p.Asp540Gly); NM_001083962.2 (TCF4): c.1849G>T (p.Val617Phe); NM_001083962.2 (TCF4): c.1741 G>T (p.Val581 Phe); NM_001083962.2 (TCF4): c.1840G>C (p.Ala614Pro); NM_001083962.2 (TCF4): c.1710G>C (p.Arg570Ser); NM_001083962.2 (TCF4): c.1147-255_1350+179del; NM_001083962.2 (TCF4): c.1166G>T (p.Arg389Leu); NM_001083962.2 (TCF4): c.1486+5G>T; NM_001083962.2 (TCF4): c.991-1del; NM_001083962.2 (TCF4): c.1871A>C (p.Gln624Pro); NM_001083962.2 (TCF4): c.1879+1G>A; NM_001083962.2 (TCF4): c.1866G>C (p.Glu622Asp); NM_001083962.2 (TCF4): c.1760C>T (p.Ala587Val); NM_001083962.2 (TCF4): c.1826T>C (p.Leu609Pro); NM_001083962.2 (TCF4): c.990G>A (p.Ser330=); NM_001083962.2 (TCF4): c.759C>G (p.Ser253Arg); NM_001083962.2 (TCF4): c.1118dup (p.Pro373_Asn374insTer); NM_001083962.2 (TCF4): c.1486G>T (p.Gly496Cys); NM_001083962.2 (TCF4): c.539del (p.Leu180fs); NM_001083962.2 (TCF4): c.1512_1513insTAGTCCAG (p.Ser505Ter); NM_001083962.2 (TCF4): c.1066_1067dup (p.Ala357fs); NM_001083962.2 (TCF4): c.1771C>T (p.Leu591 Phe); NM_001083962.2 (TCF4): c.1727G>T (p.Arg576Leu); NM_001083962.2 (TCF4): c.1454_1455del (p.Pro485fs); NM_001083962.2 (TCF4): c.560dup (p.Ser188fs); NM_001083962.2 (TCF4): c.1136dup (p.Leu379fs); NM_001083962.2 (TCF4): c.998C>T (p.Ser333Phe); NM_001083962.2 (TCF4): c.178G>A (p.Gly60Arg); NM_001083962.2 (TCF4): c.329C>T (p.Ser110Leu); NM_001083962.2 (TCF4): c.1486+2T>G; NM_001083962.2 (TCF4): c.1086del (p.Trp362fs); NM_001083962.2 (TCF4): c.1069+1052G>A; NM_001083962.2 (TCF4): c.991-2A>G; NM_001083962.2 (TCF4): c.919_922+2delinsGTCCC; NM_001083962.2 (TCF4): c.770dup (p.His258fs); NM_001083962.2 (TCF4): c.1146+1G>A; NM_001083962.2 (TCF4): c.188del (p.Gly63fs); NM_001083962.2 (TCF4): c.1171G>T (p.Glu391Ter); NM_001083962.2 (TCF4): c.1135_1138dup (p.His380fs); NC_000018.9: g.(?_53017570)_(53254347_?)del; NM_001083962.2 (TCF4): c.1481_1482insAA (p.Tyr494Ter); NM_001083962.2 (TCF4): c.1328C>G (p.Ser443Ter); NM_001083962.2 (TCF4): c.1438C>T (p.Gln480Ter); NM_001083962.2 (TCF4): c.922+5G>A; NC_000018.9: g.(?_52895456)_(53254347_?)del; NM_001083962.2 (TCF4): c.923-2A>G; NM_001083962.2 (TCF4): c.514_517del (p.Lys172fs); NM_001083962.2 (TCF4): c.1505dup (p.Gln504fs); NM_001083962.2 (TCF4): c.655+1G>T; NM_001083962.2 (TCF4): c.593_602delinsGCCGACTACAATAGGGAC (p.Ser198_Tyr201 delinsCysArgLeuGlnTer); NM_001083962.2 (TCF4): c.294del (p.Arg99fs); NM_001083962.2 (TCF4): c.1650-2A>G; NM_001083962.2 (TCF4): c.1471C>T (p.Gln491Ter); NM_001083962.2 (TCF4): c.622_628dup (p.Thr210fs); NM_001083962.2 (TCF4): c.1733G>C (p.Arg578Pro); NM_001083962.2 (TCF4): c.1498G>T (p.Gly500Ter); NM_001083962.2 (TCF4): c.791del (p.Ser264fs); NM_001083962.2 (TCF4): c.656-1G>C; NM_001083962.2 (TCF4): c.655+1_655+2dup; NM_001083962.2 (TCF4): c.415del (p.Leu139fs); NM_001083962.2 (TCF4): c.1249del (p.Asp417fs); NM_001083962.2 (TCF4): c.500-1G>A; NM_001083962.2 (TCF4): c.922+1G>T; NM_001083962.2 (TCF4): c.469C>T (p.Arg157Ter); NM_001083962.2 (TCF4): c.1777del (p.Arg593fs); NM_001083962.2 (TCF4): c.1067C>A (p.Ser356Ter); NM_001083962.2 (TCF4): c.1504C>T (p.Gln502Ter); NM_001083962.2 (TCF4): c.879del (p.Ser294fs); NM_001083962.2 (TCF4): c.1966_1969dup (p.Pro657fs); NM_001083962.2 (TCF4): c.1153C>T (p.Arg385Ter); NM_001083962.2 (TCF4): c.1144_1145insC (p.Leu382fs); NM_001083962.2 (TCF4): c.840_841insGAGAAAG (p.Ser281fs); NM_001083962.2 (TCF4): c.1570C>T (p.Gln524Ter); NC_000018.9: g.(?_53017570)_(53070769_?)del; NM_001083962.2 (TCF4): c.1957_1958del (p.Ser653fs); NM_001083962.2 (TCF4): c.1069+1G>T; NM_001083962.2 (TCF4): c.1557del (p.Asp520fs); NM_001083962.2 (TCF4): c.1239dup (p.Gly414fs); NM_001083962.2 (TCF4): c.520C>T (p.Arg174Ter); NC_000018.9: g.(?_52921708)_(53070769_?)del; NM_001083962.2 (TCF4): c.1203del (p.Asn402fs); NM_001083962.2 (TCF4): c.740dup (p.His247fs); NM_001083962.2 (TCF4): c.555T>A (p.Tyr185Ter); NM_001083962.2 (TCF4): c.696del (p.Gly232_Met233insTer); NM_001083962.2 (TCF4): c.1552G>T (p.Glu518Ter); NM_001083962.2 (TCF4): c.1719_1722dup (p.Ala575fs); NM_001243226.3 (TCF4): c.286+1G>A; NM_001083962.2 (TCF4): c.887del (p.Cys296fs); NM_001083962.2 (TCF4): c.1965dup (p.Gly656fs); NM_001083962.2 (TCF4): c.1739G>A (p.Arg580GIn); NM_001083962.2 (TCF4): c.1738C>T (p.Arg580Trp); NM_001083962.2 (TCF4): c.1733G>A (p.Arg578His); NM_001083962.2 (TCF4): c.1727G>A (p.Arg576Gln); NC_000018.9: g.(?_53128230)_(53131388_?)del; NC_000018.10: g.(?_55234528)_(55234703_?)del; NM_001083962.2 (TCF4): c.677del (p.Pro226fs); NM_001083962.2 (TCF4): c.742_743del (p.Ile248fs); NM_001083962.2 (TCF4): c.1527del (p.Ser510fs); NM_001083962.2 (TCF4): c.968C>T (p.Ala323Val); NM_001083962.2 (TCF4): c.655G>A (p.Asp219Asn); NM_001083962.2 (TCF4): c.918_922+8del; NM_001083962.2 (TCF4): c.790-2A>G; NM_001083962.2 (TCF4): c.469del (p.Arg157fs); NM_001083962.2 (TCF4): c.1699_1701 del (p.Lys567del); NM_001083962.2 (TCF4): c.1486G>A (p.Gly496Ser); NM_001083962.2 (TCF4): c.327C>A (p.Tyr109Ter); NM_001083962.2 (TCF4): c.908del (p.Thr303fs); NC_000018.9: g.(?_52888562)_53256860del; NM_001083962.2 (TCF4): c.1069+1G>C; NM_001083962.2 (TCF4): c.1681del (p.Gln561fs); NM_001083962.2 (TCF4): c.990+1G>T; NM_001083962.2 (TCF4): c.1504del (p.Gln502fs); NM_001083962.2 (TCF4): c.923-1G>A; NM_001083962.2 (TCF4): c.1146+3A>G; NM_001083962.2 (TCF4): c.762del (p.Cys255fs); NM_001083962.2 (TCF4): c.1867C>T (p.Gln623Ter); NM_001083962.2 (TCF4): c.637_639delinsCTTCATGCAACCAGCACTT (p.Ser213fs); NC_000018.9: g.(?_52927160)_(53254347_?)del; NM_001083962.2 (TCF4): c.986_990+3del; NM_001083962.2 (TCF4): c.1292del (p.Gly431fs); NM_001083962.2 (TCF4): c.1034del (p.Pro345fs); NM_001083962.2 (TCF4): c.937_941del (p.Gly313fs); NM_001083962.2 (TCF4): c.1841C>T (p.Ala614Val); NM_001083962.2 (TCF4): c.1411C>T (p.Gln471Ter); NM_001083962.2 (TCF4): c.978delinsGG (p.Ala327fs); NM_001083962.2 (TCF4): c.670del (p.Ser224fs); NM_001083962.2 (TCF4): c.795T>A (p.Tyr265Ter); NM_001083962.2 (TCF4): c.717del (p.Gly240fs); NC_000018.9: g.(?_53070665)_(53070769_?)del; NM_001083962.2 (TCF4): c.1834del (p.His612fs); NM_001083962.2 (TCF4): c.655+1G>A; NM_001083962.2 (TCF4): c.1486+1G>T; NM_001083962.2 (TCF4): c.1705C>T (p.Arg569Trp); NM_001083962.2 (TCF4): c.1720A>G (p.Asn574Asp); NM_001083962.2 (TCF4): c.1732C>T (p.Arg578Cys); NM_001083962.2 (TCF4): c.748C>T (p.Gln250Ter); NM_001083962.2 (TCF4): c.1726C>T (p.Arg576Ter); NM_001083962.2 (TCF4): c.1169del (p.Arg389_Leu390insTer); and NM_001083962.2 (TCF4): c.1876C>T (p.Arg626Ter), as identified by the National Center for Biotechnology Information (NCBI).

### II. Compositions

The composition described herein contain human UC-MSCs, isolated exosomes derived from the UC-MSCs, cell culture medium containing the UC-MSC's secretome (e.g., UC-MSC secretome-conditioned cell culture medium), cell culture medium containing the UC-MSC's secretome but devoid of exosomes (e.g., exosome-depleted UC-MSC-conditioned cell culture medium), or some combination thereof. Compositions may further include one or more Wnt pathway activators, such as any amount of a HDAC1 inhibitor (e.g., vorinostat, romidepsin, belinostat, panobinostat, valproic acid, entinostat, curcumin, quercetin, and RG2833), a Wnt-signaling agonist (e.g., L-Quebrachital), a frizzled-signaling agonist (e.g., Wnt agonist-1 or Wnt-3a), and a GSK-3B3 inhibitor (e.g., indirubin-3′-oxime, landuviglusib (CHIR-99821), and KY19382) described herein.

### A. UC-MSCs

The present disclosure provides compositions containing about 5×10⁵ to 5×10⁶ (e.g., 5×10⁵ to 1×10⁶, 5×10⁵ to 1.5×10⁶, 1×10⁶ to 2.5×10⁶, 1.5×10⁶ to 3×10⁶, 2×10⁶ to 5×10⁶, or 4×10⁶ to 5×10⁶, e.g., 5×10⁵, 6×10⁵, 7×10⁵, 8×10⁵, 9×10⁵, 1×10⁶, 1.5×10⁶, 2×10⁶, 2.5×10⁶, 3×10⁶, 3.5×10⁶, 4×10⁶, 4.5×10⁶, or 5×10⁶) isolated UC-MSCs (e.g., ALLORX STEM CELLS®). For example, a composition may contain 1×10⁶ to 2.5×10⁶ (e.g., 1.5×10⁶, 1.6×10⁶, 1.7×10⁶, 1.8×10⁶, 1.9×10⁶, 2×10⁶, 2.1×10⁶, 2.2×10⁶, 2.3×10⁶, 2.4×10⁶, or 2.5×10⁶) isolated UC-MSCs. In another example, a composition may contain about 1×10⁶ (e.g., 9×10⁵, 1×10⁶, or 1.1×10⁶) isolated UC-MSCs.

UC-MSCs may be isolated from the subject having PTHS or isolated from a healthy donor (e.g., a subject that does not have PTHS). UC-MSCs may be allogenic or autologous.

Compositions containing UC-MSCs may further include one or more Wnt pathway activators, such as any amount of a HDAC1 inhibitor (e.g., vorinostat, romidepsin, belinostat, panobinostat, valproic acid, entinostat, curcumin, quercetin, and RG2833), a Wnt-signaling agonist (e.g., L-Quebrachital), a frizzled-signaling agonist (e.g., Wnt agonist-1 or Wnt-3a), and a GSK-3B3 inhibitor (e.g., indirubin-3′-oxime, landuviglusib (CHIR-99821), and KY19382) described herein. As discussed above, the addition of one or more Wnt pathway activators may induce TCF4 gene expression (e.g., by epigenetic manipulation or signal transduction mechanisms).

Compositions containing UC-MSCs may further include a pharmaceutically acceptable carrier, excipient, or diluent and may not contain DMSO. Compositions containing UC-MSCs may further include a cryopreservation medium, a basal medium, and/or a saline solution. The cryopreservation medium may be PRIME-XV® MSC FreeziS DMSO-Free medium (e.g., FUJIFILM Irvine Scientific Catalog number: 91140). The basal medium may be MCDB-131.

UC-MSCs may endogenously express TCF4 (e.g., mRNA and/or protein) or a TCF4 fragment or isoform thereof. UC-MSCs may be modified (e.g., modified UC-MSCs) as described herein.

i. Modified UC-MSCs

UC-MSCs may be modified to have increased or prolonged TCF4 expression relative to a control (e.g., untreated UC-MSC). For example, modifying TCF4 expression in UC-MSCs can involve genetically manipulating the UC-MSCs to express exogenous (fully functional) TCF4.

UC-MSCs may be modified by introducing exogenous genetic material (e.g., DNA, cDNA, RNA, or mRNA) into the cell. For example, a nucleic acid vector, plasmid, circular RNA, or mRNA molecule may be introduced into the UC-MSCs, e.g., by way of transfection (e.g., microinjection, optical transfection, biolistic transfection, electroporation, nucleofection, lipofection, sonoporation, and magnetofection). The exogenous genetic material (e.g., nucleic acid vector, plasmid, circular RNA, or mRNA molecule) may contain a nucleotide sequence encoding a functional TCF4 (e.g., TCF4 mRNA or protein). Such exogenous expression of TCF-4 in the UC-MSCs (e.g., UC-MSCs from a healthy donor or a subject having PTHS) will augment endogenous TCF4 expression in the cell and promote the downstream signaling effects of TCF4. The exogenous genetic material (e.g., nucleic acid vector, plasmid, circular RNA, or mRNA molecule) may contain a nucleotide sequence encoding a Wnt signaling agonist. Such exogenous expression of a Wnt signaling agonist in the UC-MSCs (e.g., UC-MSCs from a healthy donor) may promote endogenous TCF4 expression in the cell, thereby promoting the downstream signaling effects of TCF4. The exogenous genetic material (e.g., nucleic acid vector, plasmid, circular RNA, or mRNA molecule) may contain a nucleotide sequence encoding a frizzled-signaling agonist (e.g., Wnt agonist-1 or Wnt 3a). Such exogenous expression of a frizzled-signaling agonist in the UC-MSCs (e.g., UC-MSCs from a healthy donor) may promote endogenous TCF4 expression in the cell, thereby promoting the downstream signaling effects of TCF4. The exogenous genetic material (e.g., nucleic acid vector, plasmid, circular RNA, or mRNA molecule) may be one or more nucleic acid molecules containing one or more nucleotide sequences (e.g., DNA, cDNA, RNA, or mRNA sequences) encoding any one or more of the following: TCF4, a Wnt signaling agonist, and a frizzled-signaling agonist (e.g., Wnt agonist-1 or Wnt 3a).

Exemplary TCF4 genes, mRNA transcript, and protein sequences to be expressed in UC-MSCs are provided in Table 1.

ii. Quality Control Standards for UC-MSC Production

Our investigational drug consists of purified, expanded and cryogenically preserved Human Umbilical Cord Mesenchymal Stem Cells in a non DMSO-containing excipient containing 50 million cells per vial at 10-12.5 million cells per mL. The donated umbilical cords are derived from American Association of Tissue Banks (AATB)-certified third-party providers. Since we use the human umbilical cord as a source of the MSCs, we have established strict criteria for the selection of full-term, donated umbilical cords for use in processing to purified ALLORX STEM CELLS®. First these tissues are only procured from AATB Accredited tissue suppliers. We manage tissue providers as per the ISO 9001:2015 quality standard and the ISO13485:2016 Medical Device Manufacturing Standard. All testing occurs using FDA approved assays.

The acceptance criteria include:
No history of sexually transmitted diseases (STD's) and tuberculosis (TB)
Freedom from risk factors for, or clinical evidence of, Zika Virus
No history of cancer or chronic illnesses
No history of autoimmunity disorders (e.g., human immunodeficiency virus (HIV), Type I Diabetes, MS, Lupus, etc.)
No tattoos within the past 12 months
No history of Creutzfeldt-Jakob disease (CJD) or prion disease
No history of drug or alcohol abuse

Tested negative by FDA-approved assays for adenovirus, Epstein-Barr (EBV), hepatitis A, hepatitis B core total antibody (Ab), hepatitis B surface antigen, hepatitis C virus Ab, human herpesvirus (HHV)-6, HHV-7, HHV-8, HIV-1/HIV-2, HTLV I/II Ab, Parvovirus 19, CMV HbSAg, HCV, HTLV I/I1, HIV I/I1, CMV, EBV, WNV, COVID-19 & Syphilis.

In addition to the selection criteria for donated umbilical cords, we perform a detailed lot specific final quality testing process before QC release of finished product including identity testing by the International Society for Cell and Gene Therapy (ISCT) phenotype standard for MSC identity: CD11b−; CD14−; CD19−; CD34−; CD44+; CD45−; CD73+; CD79a−; CD90+, CD105+; CD126−; HLA-DR− by flow cytometry. Demonstrated tri-lineage differentiation. Human karyotype and human DNA fingerprint test. Purity >95% by flow cytometry. Potency to QC release criteria by cellular ATP content and gamma-interferon induced IPO activity. Adventitious agent testing of quality includes negative 14-day USP-71 sterility testing, negative by PCR testing for 16S, 18S and Mycoplasma; negative in-vitro cell-based viral testing in Hela cells, MCR-5 and Vero76 cells, negative in-vivo viral tests in guinea pigs, post-weaning mice, suckling mice and embryonic chick embryos via yolk sack and allantoic fluid deployment. Negative human viral pathogen tests by PCR for Parvovirus B19, Polyomavirus BKV, EBV, HAV, HBV, HCMV, HCV, HEV, HHV-6, HHV-7, HHV-8, HIV-1, HIV-2, HPV-16, HPV-18, HTLV-1, HTLV-2, JCV and SARS-Cov-2.

The procedures used in manufacturing and quality control of UC-MSCs are described below in Examples 1 and 2.

### B. Isolated Exosomes

The present disclosure provides compositions containing a concentration of about 5×10⁹ to 5×10¹⁰ (e.g., 5×10⁹ to 1.5×10¹⁰, 1×10¹⁰ to 3×10¹⁰, 2×10¹⁰ to 4×10¹⁰, 3×10¹⁰ to 5×10¹⁰, 1×10¹⁰ to 5×10¹⁰, or 2.5×10¹⁰ to 5×10¹⁰, e.g., 5×10⁹, 6×10⁹, 7×10⁹, 8×10⁹, 9×10⁹, 1×10¹⁰, 1.5×10¹⁰, 2×10¹⁰, 2.5×10¹⁰, 3×10¹⁰, 3.5×10¹⁰, 4×10¹⁰, 4.5×10¹⁰, or 5×10¹⁰) isolated exosomes per mL. The present disclosure also provides compositions containing about 100 μg to about 300 pg (e.g., 100 μg to 200 μg, 125 μg to 175 pg, or 200 μg to 300 pg, e.g., 100 μg, 125 pg, 150 μg, 175 pg, 200 μg, 225 pg, 250 μg, 275 pg, or 300 pg) of the isolated exosomes.

The isolated exosomes may be derived from US-MSC secretome-conditioned cell culture medium. After removing exosomes from US-MSC secretome-conditioned cell culture medium, exosomes may contain a volume (e.g., a volume of about 2.7×10⁻¹⁰ mm³ or less) of the cell culture medium from which they were isolated from; this is still considered an “isolated” exosome.

The isolated exosomes of the present disclosure are lipid bilayer vesicles having a diameter of 80 nm to 200 nm (e.g., 80 nm, 90 nm, 100 nm, 110 nm, 120 nm, 130 nm, 140 nm, 150 nm, 160 nm, 170 nm, 180 nm, 190 nm, or 200 nm) and are isolated at purity greater than 90% (e.g., 90%, 91%, 92%, 93%, 94%, 95%, 96%, 97%, 98%, 99%, or 100%). These exosomes express at least CD9, CD63, CD81, CD44, CD29, and CD142. The isolated exosomes may not express CD45, CD11 b, CD14, CD19, CD34−, CD79a, CD126, and human leukocyte antigen-DR isotype (HLD-DR).

Compositions containing isolated exosomes may further include one or more Wnt pathway activators, such as any amount of a HDAC1 inhibitor (e.g., vorinostat, romidepsin, belinostat, panobinostat, valproic acid, entinostat, curcumin, quercetin, and RG2833), a Wnt-signaling agonist (e.g., L-Quebrachital), a frizzled-signaling agonist (e.g., Wnt agonist-1 or Wnt-3a), and a GSK-3B13 inhibitor (e.g., indirubin-3′-oxime, landuviglusib (CHIR-99821), and KY19382) described herein. As discussed above, the addition of one or more Wnt pathway activators may induce TCF4 gene expression (e.g., by epigenetic manipulation or signal transduction mechanisms).

### C. UC-MSC Secretome-Conditioned Cell Culture Medium

Compositions containing biological materials secreted from the UC-MSC (e.g., UC-MSC secretome-conditioned cell culture medium) are also envisioned for use in the treatment of PTHS. Therapeutic benefits of stem cell therapy are thought to be in part mediated by soluble factors secreted from stem cells, collectively known as paracrine effects. The secretome is also referred to as UC-MSC-conditioned medium since the manufacturing process involves collection of the cell culture medium exposed to UC-MSCs maintained in cell cultures.

The MSC secretome consists of all secreted factors, including exosomes that are lipid bilayer vesicles of 140 to 200 nm diameter comprised of integral membrane proteins including the exosome-specific biomarkers CD9, CD61 and CD83 and various biological molecules including proteins, lipids, RNA, miRNA, DNA, fats contained within exosomes combined with other soluble factors secreted from MSCs and/or exosomes. Therapy may be mediated by the large variety of secreted factors derived from the stem cell secretome.

The UC-MSC secretome-conditioned cell culture medium may contain GM-CSF, MIP-3a, IL-6, and IL-8 each at about 500-2000 pg/mL (e.g., 500-1000 pg/mL, 800-1600 pg/mL, or 1400-2000 pg/mL, e.g., 500 pg/mL, 550 pg/mL, 600 pg/mL, 650 pg/mL, 700 pg/mL, 750 pg/mL, 800 pg/mL, 850 pg/mL, 900 pg/mL, 950 pg/mL, 1000 pg/mL, 1050 pg/mL, 1100 pg/mL, 1150 pg/mL, 1200 pg/mL, 1250 pg/mL, 1300 pg/mL, 1350 pg/mL, 1400 pg/mL, 1450 pg/mL, 1500 pg/mL, 1550 pg/mL, 1600 pg/mL, 1650 pg/mL, 1700 pg/mL, 1750 pg/mL, 1800 pg/mL, 1850 pg/mL, 1900 pg/mL, 1950 pg/mL, 2000 pg/mL). The UC-MSC secretome-conditioned cell culture medium may further contain about 10 pg/mL to 500 pg/mL (e.g., 10 pg/mL to 100 pg/mL, 10 pg/mL to 500 pg/mL, or 500 pg/mL to 1000 pg/mL, e.g., 10 pg/mL, 20 pg/mL, 30 pg/mL, 40 pg/mL, 50 pg/mL, 60 pg/mL, 70 pg/mL, 80 pg/mL, 90 pg/mL, 100 pg/mL, 150 pg/mL, 200 pg/mL, 250 pg/mL, 300 pg/mL, 350 pg/mL, 400 pg/mL, 450 pg/mL, or 500 pg/mL) of fractalkine and MIP-1.

Compositions containing UC-MSC secretome-conditioned cell culture medium may further include one or more Wnt pathway activators, such as any amount of a HDAC1 inhibitor (e.g., vorinostat, romidepsin, belinostat, panobinostat, valproic acid, entinostat, curcumin, quercetin, and RG2833), a Wnt-signaling agonist (e.g., L-Quebrachital), a frizzled-signaling agonist (e.g., Wnt agonist-1 or Wnt-3a), and a GSK-3B3 inhibitor (e.g., indirubin-3′-oxime, landuviglusib (CHIR-99821), and KY19382) described herein. As discussed above, the addition of one or more Wnt pathway activators may induce TCF4 gene expression (e.g., by epigenetic manipulation or signal transduction mechanisms).

### D. Exosome-Depleted UC-MSC-Conditioned Cell Culture Medium

Exosome-depleted UC-MSC-conditioned cell culture medium is derived from UC-MSC secretome-conditioned cell culture medium with a key difference being that exosomes (e.g., exosomes that are of a specific size, e.g., 80-200 nm) have been removed. Several standard laboratory techniques exist to remove exosomes from cell culture medium, such as differential ultracentrifugation, seize exclusion chromatography, ultrafiltration, polyethylene glycol-based precipitation, immunoaffinity capture, or by using microfluidic devices. Exosome-depleted UC-MSC-conditioned cell culture medium may contain other UC-MSC-derived biological material, such as proteins (e.g., TCF4), lipids, and extracellular vesicles (EVs) smaller than 80 nm or larger than 200 nm.

Exosome-depleted UC-MSC-conditioned cell culture medium may also contain GM-CSF, MIP-3a, IL-6, and IL-8 each at about 100-2000 pg/mL (e.g., 500 pg/mL to 1000 pg/mL, 800 pg/mL to 1600 pg/mL, or 1400 pg/mL to 2000 pg/mL, e.g., 500 pg/mL, 550 pg/mL, 600 pg/mL, 650 pg/mL, 700 pg/mL, 750 pg/mL, 800 pg/mL, 850 pg/mL, 900 pg/mL, 950 pg/mL, 1000 pg/mL, 1050 pg/mL, 1100 pg/mL, 1150 pg/mL, 1200 pg/mL, 1250 pg/mL, 1300 pg/mL, 1350 pg/mL, 1400 pg/mL, 1450 pg/mL, 1500 pg/mL, 1550 pg/mL, 1600 pg/mL, 1650 pg/mL, 1700 pg/mL, 1750 pg/mL, 1800 pg/mL, 1850 pg/mL, 1900 pg/mL, 1950 pg/mL, 2000 pg/mL). The composition may further include about 10 pg/mL to 500 pg/mL (e.g., 10 pg/mL to 100 pg/mL, 10 pg/mL to 500 pg/mL, or 500 pg/mL to 1000 pg/mL, e.g., 10 pg/mL, 20 pg/mL, 30 pg/mL, 40 pg/mL, 50 pg/mL, 60 pg/mL, 70 pg/mL, 80 pg/mL, 90 pg/mL, 100 pg/mL, 150 pg/mL, 200 pg/mL, 250 pg/mL, 300 pg/mL, 350 pg/mL, 400 pg/mL, 450 pg/mL, or 500 pg/mL) of fractalkine and MIP-1.

Compositions containing exosome-depleted UC-MSC-conditioned cell culture medium may further include one or more Wnt pathway activators, such as any amount of a HDAC1 inhibitor (e.g., vorinostat, romidepsin, belinostat, panobinostat, valproic acid, entinostat, curcumin, quercetin, and RG2833), a Wnt-signaling agonist (e.g., L-Quebrachital), a frizzled-signaling agonist (e.g., Wnt agonist-1 or Wnt-3a), and a GSK-3B3 inhibitor (e.g., indirubin-3′-oxime, landuviglusib (CHIR-99821), and KY19382) described herein. As discussed above, the addition of one or more Wnt pathway activators may induce TCF4 gene expression (e.g., by epigenetic manipulation or signal transduction mechanisms).

### EXAMPLES

### Example 1: Manufacturing Methods for Production of Umbilical-Cord Derived MSCs and Exosomes

### 2D Cell Culture in Planar Monolayers:

All reagents used in processing and manufacturing are sterile filtered into autoclave (121° C. for 50 minutes) or gamma-irradiated (sterile) containers and transferred into an International Standards Organization (ISO) 7 clean room. When all materials are transferred into the ISO 7 cleanroom, sterile 70% isopropyl alcohol, sterile 3% hydrogen peroxide, and a sporocide are used to for sanitizing materials. All processing and manufacturing are done under an ISO 5 biological safety cabinet within the ISO 7 cleanroom.

The umbilical cords are collected at birth into a sterile bag and double bagged using aseptic techniques. The donor of the umbilical cord is selected through an American Association of Tissue Banks (AATB) accredited facility and serology and virology is performed prior to cord collection. The cord is delivered to Vitro Biopharma same day and quarantined until all donor testing is completed and reported. Once released, the umbilical cord is brought into an ISO 7 cleanroom. The bag containing the cord is cleaned and sanitized. It is then transferred into a sterile ISO 5 biological safety cabinet. Once processing is complete, it is plated into sterile TC-coated T-75 flasks and placed in a humidified, ISO 5 tri-gas copper incubator (5% O2/5% CO2) and expanded in cell culture medium optimized for MSC growth (Vitro Biopharma, Catalog Number SC00B2 and/or SCOOBI-SF) containing 1× penicillin/streptomycin and 25 μg/mL fungin.

Flasks are monitored for microbial growth over the isolation/purification period of 10-14 days. During the isolation/purification period, cells are washed with phosphate buffered saline (PBS) and fed with growth medium until confluency is greater than 90% in an ISO 5 biological safety cabinet. Cells are then sub-cultured using Accutase (Innovation Cell Technologies, Catalog number AT104) according to the manufacture's procedure. Cells are expanded to pass 1 for the creation of a Master Cell Bank and proceeds additional quality control testing. Additional cells are passed for expansion in TC-coated T-1000 flasks (Millipore-Sigma, Catalog Number PFHYS1008) for about 7-10 days for each pass. Sub-culture using Accutase is performed when cells reach 90% or more confluency. A Working Cell Bank is created from low passage (pass 2) cells to support additional future expansions. The Working Cell Bank is cryogenically preserved in Vitro Biopharma's cryopreservation media (CPM) and stored in liquid nitrogen (−196° C.). Additional quality control testing is performed on the lot of Working Cell Bank.

For additional expansions and finished cellular medicine, a vial from Working Cell Bank will be obtained and thawed in a 37° C. water bath with gentle agitation. The vials are wiped down with the three disinfectants and the label is collected and stored with the batch record. The vial is transferred into the ISO 5 biological safety cabinet and plated according to the standard operating instructions and placed in the ISO 5 tri-gas copper incubator (5% O2/5% CO2). Expansion takes 7-10 days with a wash and feed every 3 days. Microbial monitoring is provided at each expansion period. When bulk cellular dose is created, cells are sub-cultured, counted with viability and cryopreserved in drug master file cryopreservation medium (PRIME-XV Stem FreeziS DMSO-Free) and aliquoted into 5 mL leak-proof cryovials (12.5M cells/mL). Each vial contains 50 million cells at 4 mLs each. All cryopreserving and packaging are performed in the ISO 5 biological safety cabinet. The vials are transferred out of the clean room into a controlled rate freezer for the freezing period (−1° C./min). Once cells reach−80° C., the vials incubate for 15 minutes at −80° C. and are transferred into liquid nitrogen (−196° C.) for quarantine storage prior to QC testing and release.

### 3D Cultures on Microcarriers in Stirred Tank Bioreactors:

There are potential advantages of 3D cultures compared to 2D including: a) Microcarrier biochemical and biophysical properties, including higher surface area/volume ratios and culture conditions can be optimized for high density cultures, b) Stirred tank bioreactors and associated bioprocessing steps are automated in closed, sterile systems readily available commercially that allow consistent control of the culture environment, c) Process probes allow online, real time monitoring of crucial cell culture conditions including cell density allowing immediate corrections if needed, d) These systems are scalable from laboratory, pilot scale to production levels with few scale-up obstacles, e) Potential cell damage due to mechanical forces such as shear can be obviated, f) The cost of production can be reduced compared to planar culture systems without sacrifice in product quality. (M. May, Microcarrier-based bioreactors can make more stem cells, GEN Feb. 22, 2022).

We performed initial small scale experiments to optimize microcarrier type and basic culture conditions in shaker flasks. We optimized binding and single passage expansion by using binding conditions resulting 2-3 cells per microcarrier and low serum levels initially (0.05%) in MSC-Gro (Vitro Biopharma, Inc. Catalog Number SC00B4) that were increased to 5% at 4-6 hours post-inoculation. The microcarrier concentration was 7,935 microcarriers per mL in the SC00B4 culture medium and the cultures were maintained in 5% O2/5% CO2 gas phase in an humidified cell culture incubator with continuous agitation at 55 RPM resulting in just suspended microcarriers. Cultures were inoculated with 0.952×106 cells in 40 ml SC00B4. Table 2 shows the cell counts and viability.

TABLE 2
Cell Counts and Viability at 7 Days Post-Inoculation

| | | | | | | | | Fold |
|---|---|---|---|---|---|---|---|---|
| | | | | | | | | increase |
| | | | | | | | | from |
| | Count 1 | Count 2 | Count 3 | Avg | [x] | Total | StdDev | inoculation |
| Fact III | | | | | | | | |
| Cells | 4.15E+05 | 2.58E+05 | 1.85E+05 | 2.86E+05 | 8.58E+05 | 3.43E+07 | 117528.7 | 36.00 |
| Viability | 98.00% | 97.40% | 95.10% | 96.83% | 96.80% | | 0.015308 | |
| Collagen | | | | | | | | |
| Cells | 4.90E+05 | 4.00E+05 | 3.41E+05 | 4.10E+05 | 1.23E+06 | 4.92E+07 | 75035.55 | 51.7 |
| Viability | 96.10% | 93.80% | 95.30% | 95.07% | 95.00% | | 0.011676 | |

These results show an average 44-fold increase in cell count during 7 days culture within the shaker flasks and the MSC cell viability was greater than 95%. This is a considerable increase over the similar expansion in planar, 2D flasks that is typically 10-fold or less.

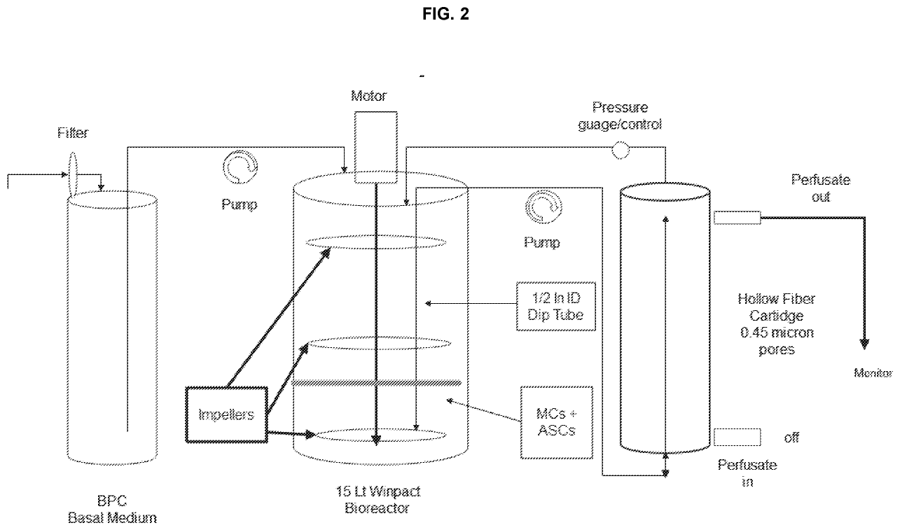

Also, FIGS. 2 & 3 show diagrams of the procedure to collect ALLOEX EXOSOMES® from stirred tank bioreactors using the inoculation conditions described above. Diafiltration as shown in FIG. 2, is automated dialysis that is used to exchange the growth medium with basal medium needed to produce the initial MSC-derived product, exosome-containing conditioned medium (ALLOEX EXOSOMES®). This product has commercial applications in cosmetics and development of regenerative medicine biologics derived from exosomes and other products secreted from cultured MSCs.

Initially, the impellers of the bioreactor that maintain a homogeneous mixture of MSCs (ALLORX STEM CELLS®) attached to microcarriers in growth medium are turned off allowing the MC/ASC complex to settle to the bottom of the bioreactor as illustrated below the blue line. This happens at the transition of the growth curve from exponential to the plateau phase as determined by a capacitance monitoring probe, about 8 to 10 days following inoculation and continuous culture in the bioreactor. This mixture is then diluted % or less in basal medium to reduce viscosity and pumped out of the bioreactor and through the 0.45 micron hollow fiber cartridge without applied back pressure and back into the bioreactor. Back pressure is then applied to the HF cartridge outlet to create cross-flow filtration and create perfusate outward flow. The flow rate from the basal medium BPC into the bioreactor is set to equal the perfusate outflow rate from the HF cartridge. The process continues until 5 to 6 volumes of basal medium has been pumped through the system, which is sufficient to exchange the growth with basal medium in the culture consisting of UC-MSCs attached to microcarriers. Critical process parameters include fluid flow dynamics (flow rates, tubing materials & dimensions) to prevent fouling, use of diaphragm pumps for MC/stem cell complexes to optimize stem cell viability and sterility in all connections/tubing and BPCs.

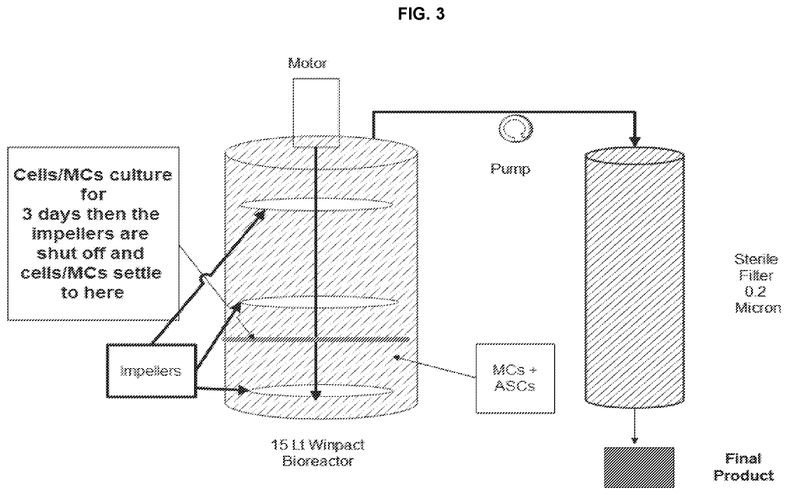

Following diafiltration, the bioreactor is then filled with basal media and continuous cell culture proceeds for about three days that allows for the secretion of various growth factors and other paracrine products of MSCs, i.e., the scretome together with MSC-derived exosomes. FIG. 3 shows the process of harvest of the conditioned medium. The impellers are shut off allowing the Stem cell-microcarrier complexes to settle and separate from the conditioned medium that is then pumped out and through a sterilizing 0.2 micron filter cartridge into an appropriate Bioprocessing container as final product.

### Example 2: Quality Control and QC Release of ALLORX STEM CELLS®

Final product QC involves culture of a cell sample taken from the final product and plated on a Tryptic Soy Agar (TSA) Plate (Hardy Biologics, catalog number P34), Sabouraud Dextrose Agar (SDA) Plate (Hardy Diangostic, catalog number P36) and a Brucella Blood Agar (BBA) Plate (Anaerobe Systems, catalog number AS-141) followed by incubation at 37° C. for 72 hours. Unites States Pharmacopeia (USP)<71> sterility testing is performed followed by a 14 day incubation period. Negative results on plates and USP <71> are QC release criterion.

USP <63> Mycoplasma testing is performed. The absence of Mycoplasma is determined through validated PCR assay system (eMyco Plus Mycoplasma PCR kit, Intron, catalog number 25234). This system is validated to detect over 200 known species of Mycoplasma. A negative result is required to pass QC release.

Absence of bacteria is determined through PCR of 16S ribosomal RNA (Fast MicroSeq 500; Applied Biosystems). This system is validated to detect over 2000 species of bacteria. A negative result is required to pass QC release.

Absence of fungi is determined through PCR of 18S ribosomal RNA (MicroSeq D2 LSU, Applied Biosystems). This system is validated to detect over 1100 species of fungi. A negative result is required to pass QC release.

USP <85> Endotoxin Limulus Amebocyte Lysate (LAL) method is performed having an Endotoxin levels less than 0.25 EU/ml by a LAL method (ThermoFisher, catalog number A39552). The procedures described for Chromogenic LAL endotoxin conforms with those described in the FDA Guidelines. This threshold level is based on recommended levels by the FDA based on lymphatic and cardiovascular exposure levels (Guidance for Industry: Pyrogen and Endotoxin Testing; DHHS, FDA, June 2012).

Since this product may be used for intravenous drip injection into patients undergoing clinical trials, we have set our criterion for acceptance at 5-fold less than industry standard as additional assurance of sterility/absence of contamination. In process safety QC involves monitoring of microbes and sterility testing (TSA, SDA, BBA, TSB, FTM, 16S PCR, 18S PCR, Mycoplasma PCR, Chromogenic LAL Endotoxin).

### USP-71 Sterility Testing Uses:

1.In Tryptic Soy Broth (TSB), inoculate 500 μL of cells and positive controls (Staphylococcus aureus [ATCC 6538], Pseudomonas aeruginosa [ATCC 9027], Bacillus subtilis [ATCC 6633], Candida albicans [ATCC 10231], Aspergillus brasiliensis [ATCC 16404]) into a 5 mL TSB tube and place in rack at room temperature in cabinet. Incubate for 14 days at 20-25° C. Check at Day 1, 3, 5, 7, 14. Have a negative control. Obtain pictures.

2. In Fluid Thioglycollate Broth (FTB), inoculate 500 μL of cells and positive controls (Staphylococcus aureus [ATCC 6538], Pseudomonas aeruginosa [ATCC 9027]) into a 5 mL FTB tube and place in rack at room temperature in cabinet. Incubate for 14 days at 35-37° C. Check at Day 1, 3, 5, 7, 14. Have a negative control. Obtain pictures.

Human viral pathogen testing occurred by DNA sequence analysis and quantitation of specific viral nucleic acid sequences by fluorescent probe technology according to Good Manufacturing Practice (GMP) regulations found in Title 21 C.F.R. Parts 210 & 211 by a third party CRO. The human viral pathogens tested are: Parvovirus B119, Polyomavirus BKV, EBV, HAV, HBV, HCMV, HCV, HEV, HHV-6, HHV-7, HHV-8, HIV-1, HIV-2, HPV-16, HPV-18, HTLV-1, HTLV-2, JCV and SARS-Cov-2. QC release criteria requires negative test results from each viral PCR test.

In-vitro cell-based assays for viral pathogens use Hela Cells, MCR-5 and Vero76 cells tested for cytopathic effect, Hemadsorption test and Hemagglutination test according to GMP regulations found in Title 21 C.F.R. Parts 210 & 211 by a third party CRO. QC release criteria requires negative results from final product and positive results for the positive control Bovine Parainfluenza 3 virus.

In-vivo adventitious virus assay for detection of inapparent viruses in biological samples is determined according to Guidance for Industry, February 2010, Characterization and Qualification of Cell Substrates and Other Biological Materials Used in the Production of Viral Vaccines for Infectious Disease Indications, European Pharmacopoeia 2.6.16, Tests for Extraneous Agents in Viral Vaccines for Human Use Points to Consider in the Characterization of Cell Lines Used to Produce Biologicals as recommended by the US FDA Center for Biologics Evaluation and Research (1993) International Conference on Harmonization, Guidance for Industry Q5A (R1): Viral Safety Evaluation of Biotechnology Products Derived from Cell Lines of Human or Animal Origin (1999), US and EU regulations was performed in guinea pigs, post-wean and suckling mice and the embryonic chick embryo by a third party CRO.

All animals and eggs assigned to this protocol were obtained from the CRO production facilities on which routine health monitoring was performed. The Test Article was received from the Client and was inoculated via multiple routes into guinea pigs (Hartley, 350-450 grams), mice (PWM, CD-1, 15-20 grams, and suckling <24 hours) and embryonated chicken eggs (10-11 days for allantoic fluid inoculation and 6-7 days for yolk sac inoculation); the hosts were monitored. After the completion of the prescribed observation period, survival percentages were determined. Guinea pigs were submitted for gross necropsy. Appropriate specimens from the suckling mice and embryonated eggs were processed, and hemagglutination testing was performed on allantoic and yolk sac fluids. Additionally, homogenates or pools from primary inoculation groups of suckling mice and embryonated chicken eggs were passaged into secondary inoculation groups of mice and eggs. The secondary inoculation groups were monitored. The survival percentage of each secondary inoculation group was determined at the completion of the observation period and hemagglutination testing was performed on the designated specimens.

QC release criteria requires negative results from final product and positive results for all positive controls. Any unexpected results were repeated and determined to be of non-cellular origin.

Purity and identity were determined by testing to International Society for Cell and Gene Therapy (ISCT) standards for MSC definition:
Adherence to plastic, phenotype consisting of: CD11 b−; CD14−; CD19−; CD34−; CD44+; CD45−; CD73+; CD79a−; CD90+, CD105+; CD126−; HLA-DR− by flow cytometry.
Demonstrated tri-lineage differentiation (e.g., differentiation into bone, cartilage, and fat cell). Human karyotype and human DNA finger print test.
Purity >95% by flow cytometry.
Potency to QC release criteria by cellular ATP content and gamma-interferon induced IDO activity.

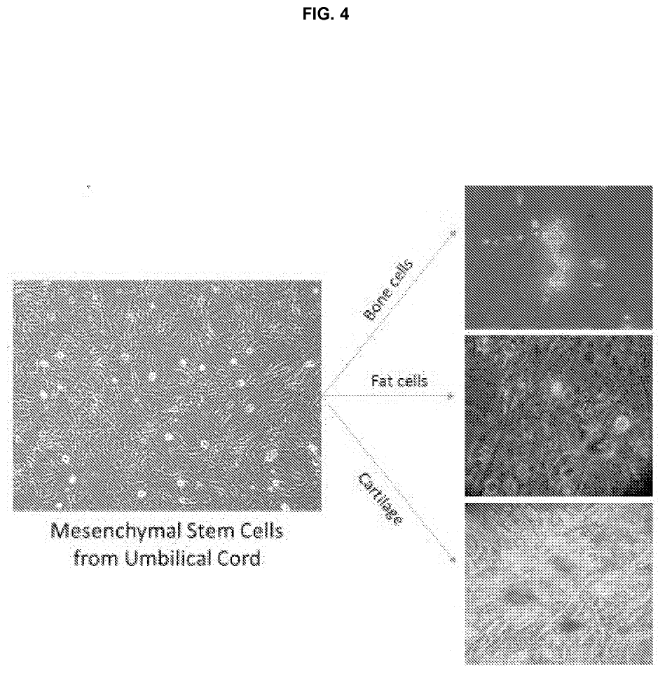

QC release criteria include required identity by phenotypic marker flow cytometry analysis, trilineage differentiation, human karyotype and human DNA test results, purity >95% and potency by criterion levels of ATP cellular content and IDO levels. See FIG. 4.

### Example 3: Exosome-Containing Conditioned Medium (AlloEx Exosome) Analysis

Exosomes within ALLOEX EXOSOMES® were first purified by size exclusion chromatography that resolves particles by size. Larger particles elute first on the column, followed by proteins and small molecular weight compounds. An Izon 35 nm qEV SEC column was pre-equilibrated with 20 mL of freshly filtered PBS. With cap closed, the buffer from the top of the column was removed and 500 μL of the sample was loaded. Cap was opened immediately and 0.5 mL fractions were collected. The column was not allowed to dry out at any time, and fresh PBS was added at the top when needed to maintain the flow. First 6 fractions (3 mL)—void volume, were discarded. Exosome fractions 7, 8 and 9 were collected and pooled together. The exosome fractions were concentrated using Amicon Ultra 0.5 30 kDa MWCO centrifugal filter devices. A total of 50 pL was recovered after centrifugation and transferred into a new tube. The filter membranes were rinsed with 100 μL of PBS by pipetting up and down 10 times. The wash buffer was combined with the retained exosomes and total of about 150 μL of exosomes in PBS was collected for further analysis.

Fluorescent NTA technique involves labeling of intact exosomal membrane with a fluorescent dye and then performing the analysis in scatter and fluorescent modes. This technique allows exclusion of contaminant particles, such as protein aggregates, lipoproteins, etc from analysis and assessment of the purity of exosome sample. The analysis was performed with Zetaview (Particle Metrix) instrument equipped with 520 nm laser, 550 nm long pass cut off filter and sCMOS camera. DI water was filtered on the day of analysis through 0.22 μm syringe filter and its purity confirmed by NTA prior to the study. Exosome labeling was done using Exoglow fluorescent NTA labeling kit from System Biosciences according to manufacturer's protocol. Briefly, 12 μL of reaction buffer were mixed with 2 μL of dye and 36 μL of sample. The mixture was vortexed for 15 seconds to mix well and samples were incubated at RT for 10 minutes. Liposomes (provided with kit) were used as labeling control: 1 μL of liposomes was mixed with 12 μL of reaction buffer and 2 μL of dye. Dilutions were made by mixing DI water filtered through 0.2 μm syringe filter with corresponding volume of a sample.

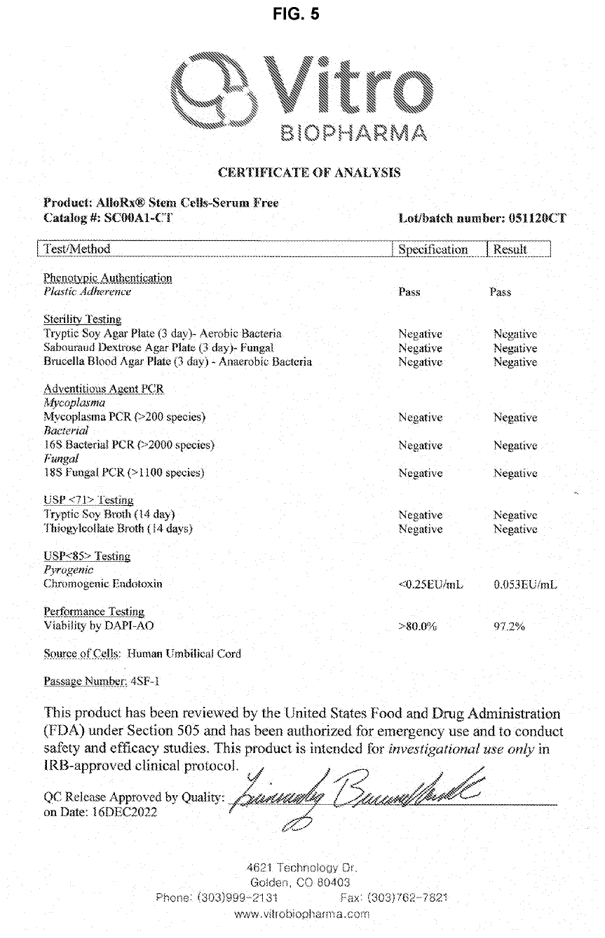

The results are shown in FIG. 5. Lot 021422 ALLOEX EXOSOMES® (Exosome-containing Conditioned medium) contained 69 billion exosomes/ml that averaged 165 nm in diameter at 94.2% purity and were positive for exosome biomarkers: CD9 (21.7%), CD63 (21.7%) and CD81 (6%), indicating identity as exosomes, although strict standards of exosome identity, purity and potency have not yet been established.

### Example 4: Enhanced Secretion of Heat Shock Protein 70 (Hsp70) from UC-MSCs by Exposure to Lithium

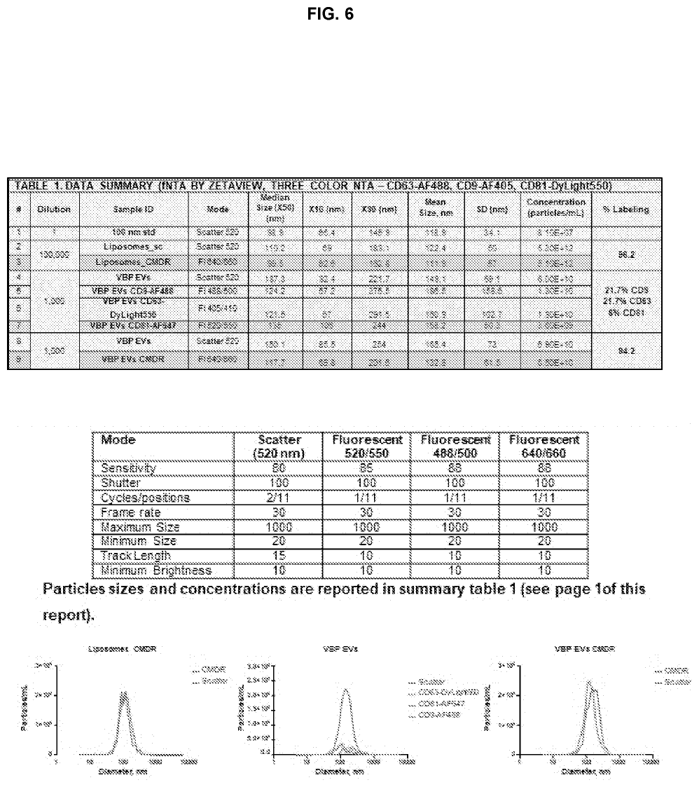

FIG. 6 shows secretion of Hsp70 as a function of LiCl concentration. UC-MSCs were plated at 5,000 cells/cm² in T-25 flasks and cultured until confluence was 80-90%. The cells were then exposed to the LiCl concentrations shown on the x axis and the medium was collected 96 hours later and assayed by immunoassay for Hsp70. The results show significant increased secretion of Hsp70 at 200 micromolar LiCl (p<0.05). Since Hsp70 mediates cellular protection as a chaperone molecule and through antiapoptotic mechanisms (refs), its enhanced secretion from MSCs (possible through inclusion in exosomes Ref) may reflect mechanisms of stem cell activation by GSK-3p inhibition in conjunction with HDAC inhibition (e.g., see U.S. application Ser. No. 17/239,513 which is incorporated by reference). This effect is a possible mode of action of the therapy proposed by the present disclosure.

### Example 5: Clinical Trial Results Using ALLORX STEM CELLS®

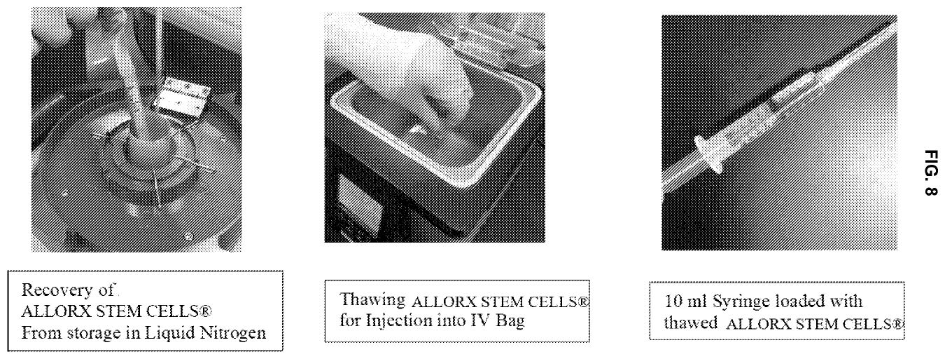

Table 3 below shows the results of various clinical trials performed using ALLORX STEM CELLS®s manufactured and subjected to quality control procedures as described above. These studies were overseen by IRBs using approved protocols and administration of varying dosages of ALLORX STEM CELLS®. FIG. 8 describes the process of preparation of the cells for IV infusion or direct injection.

TABLE 3
Results of Various Clinical Trials

| | | | Dose/ | Dosing | |
|---|---|---|---|---|---|
| | | | No. of | Regimen/ | |
| Mode of | Study | # of | Cells | Duration of | Summary of |
| Admin. | Population | Subjects | (million) | treatment | Safety |
| i.v. | AD | 3 | 300m per | 1 (once) | No SAE's or AE's (self- |
| | | | treatment | | resolving AE's if any) |
| i.v. | ALS | 14 | 100m per | 1-3 times | No SAE's or AE's (self- |
| | | | treatment | | resolving AE's if any) |
| i.v. | Autism | 3 | 100m-300m | 1 (once) | No SAE's or AE's (self- |
| | | | per treatment | | resolving AE's if any) |
| i.v. | Crohn's | 5 | 300m per | 1 (once) | No SAE's or AE's (self- |
| | Disease | | treatment | | resolving AE's if any) |
| i.v. | Chronic | 3 | 300m per | 1 (once) | No SAE's or AE's (self- |
| | Kidney | | treatment | | resolving AE's if any) |
| | Disease | | | | |
| i.v. | COPD | 7 | 300m per | 1 (once) | No SAE's or AE's (self- |
| | | | treatment | | resolving AE's if any) |
| i.v. | Diabetes | 6 | 100m per | 1 (once) | No SAE's or AE's (self- |
| | | | treatment | | resolving AE's if any) |
| i.v. | Long COVID \| | 33 | 150m per | 1 (once) | No SAE's or AE's (self- |
| | COVID | | treatment | | resolving AE's if any) |
| i.v. | Lupus \| Lyme \| | 119 | 150m or | 1-2 times | No SAE's or AE's (self- |
| | RA \| | | 300m/per | | resolving AE's if any) |
| | Fibromyalgia | | treatment | | |
| | Inflammatory | | | | |
| i.v. | Multiple | 43 | 300m per | 1 (once) | No SAE's or AE's (self- |
| | Sclerosis | | treatment | | resolving AE's if any) |
| i.t. or | Osteoarthritis | 30 | 100m to | 1-3 times | No SAE's or AE's (self- |
| i.m. | | | 300m | | resolving AE's if any) |
| | | | depend on | | |
| | | | site | | |
| i.v. | Parkinson's & | 10 | 300m per | 1 (once) | No SAE's or AE's (self- |
| | Muscular | | treatment | | resolving AE's if any) |
| | Dystrophy | | | | |
| i.v. | Other | 26 | Varies | Varies | No SAE's or AE's (self- |
| | | | | | resolving AE's if any) |

TOTAL = 301 Patients

These trials were Phase I/II open label, non-randomized, nor placebo controlled thus there are no direct comparisons with non-treated patients. The efficacy results were mainly anecdotal reports of symptom remission by patients. For example, a case study of a multiple sclerosis (MS) patient treated illustrates this efficacy. This MS patient was treated by three successive infusions of 300 million MSCs. The first treatment used autologous adipose-derived MSCs and resulted reduction of neurological symptoms for 4 months followed by relapse to pre-treatment levels of neurological function, as reported by the patient. Approximately 4 years later, this patient received two successive IV infusions of 300 million ALLORX STEM CELLS&. The initial treatment—as reported by patient-provided relief and a significant decrease in neurological symptoms (Left leg foot drop) for 18 months succeeding the treatment. Subsequently, the patient underwent another IV infusion therapy of 300 million ALLORX STEM CELLS® and reported further improvements in neurological symptoms including restoration of thermal sensory function in his left arm and a dramatic increase in energy level compared to the normal daily fatigue that is a common feature of MS. This patient received a cross-over protocol design in that the initial dosage was autologous adipose-derived MSCs while the subsequent two treatments consisted the allogeneic umbilical cord-derived MSCs (e.g., ALLORX STEM CELLS®). Since in-vitro results showed significantly increased potency of umbilical cord MSCs (ALLORX STEM CELLS®) over adipose-derived MSCs by both mitochondrial and immunosuppression, the clinical outcomes corroborate the potency measurements by cellular ATP levels and gamma IFN-induced IDO activity. Thus, significantly greater potency of UC-MSCs yields longer, more sustainable remission of MS symptoms than adipose-derived MSCs.

### Example 6: Comparative Analysis of Adult Mesenchymal Stem Cells Derived from Adipose, Bone-Marrow, Placenta, and Umbilical Cord Tissue

Mesenchymal stromal/stem cells (MSCs) have the potential to repair and regenerate damaged tissues, making them attractive candidates for cell-based therapies. Expanded and well-characterized MSCs have application in regenerative medicine and have been used in several clinical trials including treatment for osteoarthritis and other conditions. Here, we provide results of a comparative study of purified and expanded MSCs from adipose, bone-marrow, placenta, and umbilical cord involving determination of phenotype by flow cytometry analysis, cellular potency by quantitative assessment of mitochondrial function and immunosuppression, and cellular function by quantitative assessment of cell migration and proliferation. Our results show comparable phenotypic profiles, morphology, expansion in cell culture and adipogenic, osteogenic and chondrogenic differentiation. Potency measures of mitochondrial/immunosuppressive capacity and additional cellular function assays show differences suggesting biological advantages of umbilical MSCs.

Mesenchymal stem cells (MSCs) are multipotent, non-differentiated adult stem cells capable of self-renewal, proliferation, conversion into differentiated cells as well as the regeneration of tissues. MSC-based regenerative medicine offers novel therapies for patients with injuries, end-stage organ failure, degenerative diseases, and several other medical conditions. Transplanted MSCs have shown potential therapeutic benefits and safety in myocardial, musculoskeletal, neurological, autoimmune disorders, and several other disorders (Carralho E, et al. Regen. Med. 2015; 10:1025; Freitag J, et al. BMC Musculoskeletal Disorders 2016; 17: 230; Neirinckx V, et al. Stem Cell Trans. Med 2013; 2: 284; Munir H and McGettrick, HM Stem Cells Dev. 2015; 24:2091). MSCs are isolated from several tissues including lipoaspirates, perinatal tissues, cord blood, teeth, etc. and have considerable capacity for in vitro expansion and broad regenerative potential. These properties make MSCs attractive candidates for cell-based therapies.

No MSC-based therapies are yet approved for clinical application in the US while hematopoietic stem cells are FDA-approved for clinical use. The European Medicines Agency has recently approved allogeneic MSCs (Alifosel™) derived from adipose-tissue for treatment of a type of Crohn's disease. Other countries have different regulatory requirements for commercial approval of stem cell therapies.

Clinical trials based on expanded MSCs are common internationally, although there are variations in the degree of regulation including the requirements for adherence to cGMP standards. Here, we report on phenotypic and functional characterization of purified and expanded MSCs from adipose, bone-marrow, placenta, and umbilical cord. Our results show comparable growth and tri-lineage differentiation performance while umbilical cord MSCs display enhanced potency, cellular functions and capacity for differentiation into neural stem cells.

### Results

### Growth in Cell Culture

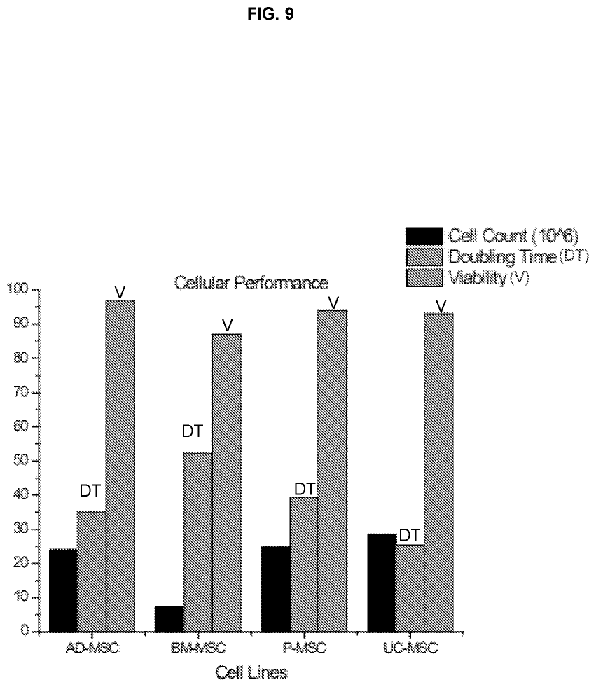

We initially compared the growth and expansion characteristics of AD-MSCs, BM-MSCs, P-MSCs and UC-MSCs following pass 2 in cell culture as described above. The results shown in FIG. 9 at pass 2 show doubling time (Td) between 20 hours for UC-MSCs and 53 hours for BM-MSCs while MSC viability was relatively consistent at 85 to 95%. This shows comparable expansion at variable growth rates: UC-MSC>AD-MSC>P-MSC>BM-MSC. Similar results were seen in several replicates (n=4) of this protocol.

### Phenotypic Characterization of AD-MSCs, BM-MSCs, P-MSCs, and UC-MSCs

The isolated and expanded AD-MSCs, BM-MSCs, P-MSCs, and UC-MSCs were investigated for MSC phenotype at P2 by staining for cell surface markers, which were detected using flow cytometry according to the ISCT standard (Dominici M, et al. Cytotherapy. 2006; 8: 315) and the results are shown in Table 4. The AD-MSCs and UC-MSCs expressed the typical MSC markers CD90, CD73, and CD105. In addition, the cells showed low expression of hematopoietic markers CD11 b, CD14, CD19, CD34, CD45, and the MHC class II molecule HLA-DR. Similar results have been seen in several replicates (n=4). However, the P-MSCs expressed a high level of CD45, possibly due to leukocyte contamination. The BM-MSCs also expressed higher levels of CD45 and CD79a, possibly due to residual levels of B-cells.

TABLE 4
Summary results of flow cytometry analysis of UC-MSCs, P-MSCs, AD-
MSCs & BM-MSCs compared to the ISCT standard definition of an MSC

| Cell Surface Markers | ISCT Standard | UC-MSC | P-MSC | AD-MSC | BM-MSC |
|---|---|---|---|---|---|
| CD11 | Negative | Negative | Negative | Negative | Negative |
| CD14 | Negative | Negative | Negative | Negative | Negative |
| CD19 | Negative | Negative | Negative | Negative | Negative |
| CD34 | Negative | Negative | Negative | Negative | Negative |
| CD45 | Negative | Negative | Positive | Negative | Positive |
| CD73 | Positive | Positive | Positive | Positive | Positive |
| CD79a | Negative | Negative | Negative | Negative | Positive |
| CD90 | Positive | Positive | Positive | Positive | Positive |
| CD105 | Positive | Positive | Positive | Positive | Positive |
| HLA-DR | Negative | Negative | Negative | Negative | Negative |

Positive values are >90% and negative are <5%

### Immunomodulatory Potency Measures of AD-MSCs, BM-MSCs, P-MSCs, and UC-MSCs

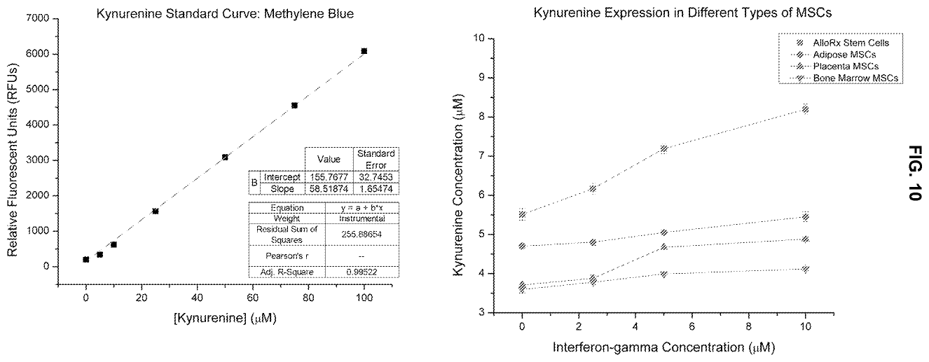

To compare immunomodulatory properties of MSCs from various sources, the activation of IDO by exposure to γ-IFN was determined on an equivalent cellular basis (FIG. 10). The γ-IFN-induced IDO activity was quantified by the conversion of tryptophan to kynurenine. Maximal IDO activity at 10 ng/ml γ-IFN was ˜4 fold greater in the isolated and expanded UC-MSCs versus other MSCs derived from other tissues. These results show greatest immunomodulatory cellular potency in expanded UC-MSCs followed by AD-MSCs, P-MSCs, and BM-MSCs. There was a significant difference in γ-IFN-induced IDO activity between the AD-MSCs, BM-MSCs, and P-MSCs compared to UC-MSCs with a p-value<0.005 by one-way ANOVA analysis (Graph Pad Prism™) of variance for significance of slope difference.

### Mitochondrial Function Analysis of AD-MSCs, BM-MSCs, P-MSCs, and UC-MSCs

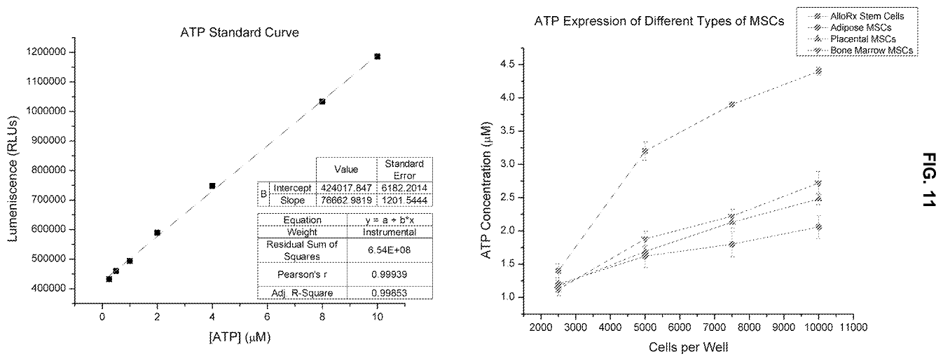

Potency was also measured by cell-specific ATP determination as previously used to determine potency of human HSCs & MSCs (Harper, H and Rich, IN, Methods Mol Biol. 2015; 1235:33; Deskins D, et al., Stem Cells Transl Med. 2013; 2:151) Relative luminescent units were converted to [ATP] using the ATP standard curve (Left panel, FIG. 11) and cellular ATP is shown as a function of cells per well (Right panel, FIG. 11). Cellular potency is measured by the slope of this relation (Harper, H and Rich, IN, Methods Mol Biol. 2015; 1235:33; Deskins D, et al., Stem Cells Transl Med. 2013; 2:151) and UC-MSCs showed greater potency than expanded AD-MSCs, BM-MSCs, and P-MSCs. There was a significant difference between the AD-MSCs, BM-MSCs, and P-MSCs compared to the isolated and expanded UC-MSCs with a p-value >0.05 by one-way ANOVA analysis (Graph Pad Prism™) of variance for significance of slope difference.

### Comparison of Cell Migration by AD-MSCs, BM-MSCs, P-MSCs, and UC-MSCs

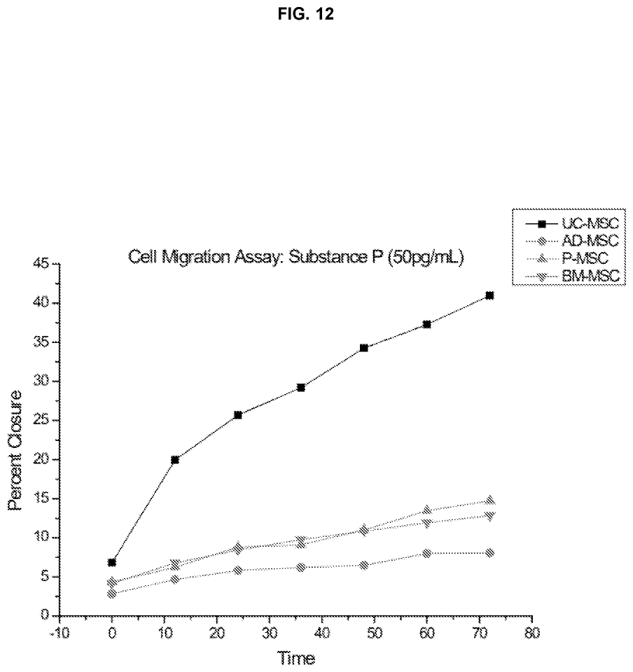

Since MSCs are well known to migrate to sites of inflammation, injury and to cancer stem cells, we compared the migration of AD-MSCs, P-MSCs, BM-MSCs and UC-MSCs in response to exposure to Substance P, a multi-functional neuropeptide. The results show in FIG. 12 show the relative migration measured as a per-cent closure of the occluded plate region following exposure to 50 pg/ml Substance P. UC-MSCs showed greatest closure at 50 pg/mL substance P (˜40% closure), while AD-MSC, P-MSC, and BM-MSC had a closure between 5-15%. These results were seen in several replicates (n=4).

### Cell Proliferation Analysis of AD-MSCs, BM-MSCs, P-MSCs, and UC-MSCs

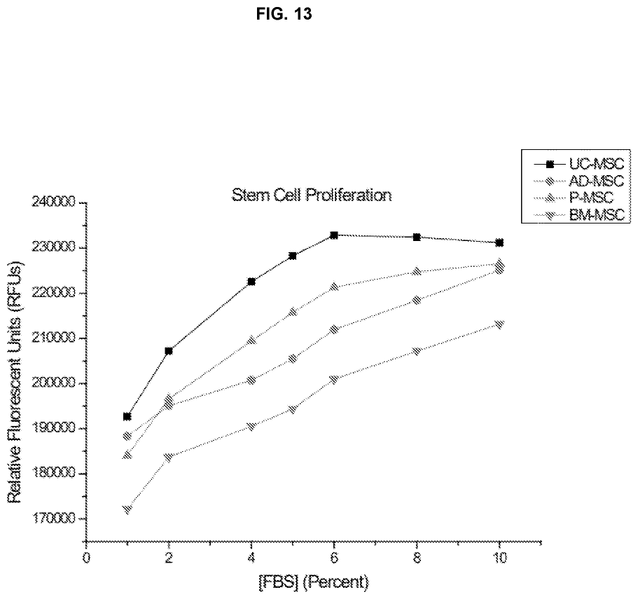

We also compared proliferation capacity of AD-MSCs, P-MSCs, BM-MSCs and UC-MSCs by quantifying cellular redox activity by a well-validated resazurin-based fluorometric assay. FIG. 13 shows the results of the comparison of proliferation by AD-MSCs, P-MSCs, BM-MSCs and UC-MSCs. The relative fluorescent difference at day 1 and 3 using Presto Blue as shown as a function of FBS content in a serum-free medium. These results were seen in several replicates (n=4). UC-MSC had a maximum effect of FBS at 6%, while no saturation was seen in the other cell lines.

### Comparison of Differentiation Capacity

We also compared functional differentiation of AD-MSCs, P-MSCs, BM-MSCs and UC-MSCs. First, we determined tri-lineage differentiation into adipocytes, chondrocytes and osteoblasts. We used standard methods that showed equivalent differentiation between the MSCs derived from adipose, placental, bone marrow and umbilical tissues (data not shown). We also investigated differentiation into neural stem cells and the results of IHC marker expression are shown in Table 5. The markers Nestin, 3PDGH, GLAST, p33-Tubulin, MAP2 & Neurofilament M are specific to neural stem cells (Wu, R, et al, Cell Biol Int 2013; 37: 812) and while the various MSCs tested were positive for most markers, the P-MSCs and AD-MSCs were negative for GLAST while this antigen was expressed on cells derived from UC-MSCs as well as the control NSCs (hNSC). This suggests a difference in differentiation capacity in that UC-MSCs can fully differentiate into the NSC phenotype while AD-MSCs and P-MSCs do not using our differentiation protocol. This does not necessarily indicate a lack of capacity of P-MSCs or AD-MSCs to differentiate into NSCs.

TABLE 5
IHC Marker Expression

| | | | | βIII- | | Neurofil- |
|---|---|---|---|---|---|---|
| | Nestin | 3PDGH | GLAST | Tubulin | MAP2 | ament M |
| AD-MSC | Positive | Positive | Negative | Positive | Positive | Positive |
| P-MSC | Positive | Positive | Negative | Positive | Positive | Positive |
| UC-MSC | Positive | Positive | Positive | Positive | Positive | Positive |
| hNSC | Positive | Positive | Positive | Positive | Positive | Positive |

### Discussion

In the present study, we compared the cellular phenotype, potency, and functionality of expanded MSCs from different sources. Expanded MSCs were derived from lipoaspirate, bone marrow, placental decidua basalis, and Wharton's jelly of the umbilical cord. Our results showed expanded MSCs share universal properties, such as morphology, plastic adherence, and multi-lineage differentiation potential. We found variations between AD-MSCs, BM-MSCs, P-MSCs, and UC-MSCs in terms of growth rate, phenotypic characterization, potency, and functionality measurements.

We used quantitative assays to determine cell counts, viability, phenotype, potency by immuno-modulatory and mitochondrial function, and functionality by migration and proliferation. Variability in measurement was minimized by careful adherence to standard procedures including processing, analysis, and expansion. Additionally, each assay was performed at the same passage to avoid variation due to differences in passage number (Javazon E H, et al, Exp Hematol. 2004; 32:414).

International criteria of MSC identity was determined by flow cytometry according to ISCT standards (Dominici M, et al. Cytotherapy. 2006; 8: 315). Placental MSCs and bone-marrow MSCs did not achieve ISCT criterion values of CD45 and CD79a. The increased expression of CD45 in P-MSCs may be due to associated leukocytes and CD79a from residual B-cells.

Cellular potency is an important assessment of stem cells for clinical applications. We used quantitative assessment of mitochondrial function and immunosuppression as measures of cellular potency. Since MSCs are intrinsically immunosuppressive in nature, they can support graft survival and other clinical effects based on immunosuppression (Liu, R, et al., Stem Cells Dev 2013; 22:1053; Wang, L T, et al, J Biomed Sci 2016; 23: 76). However, the failure of MSCs to elicit immunosuppression is likely due to immune enhancing effects of MSCs triggered by proinflammatory cytokines, educed NO, etc while IDO expression induces immunosuppressive effects of MSCs. IDO has been proposed as a molecular switch to induce immunosuppression in MSCs (Li, W et al, Cell Death & Differentiation 19: 1505, 2012). We thus determined cellular potency by quantitation of γ-IFN induced IDO activity. The results showed maximum immunomodulatory potency in UC-MSCs, which was significantly greater than MSCs sourced from other tissues (FIG. 10). This compares with other studies. Wang, Q, et al, (Human Vaccin & Immunother 12: 85, 2016) compared fetal BM derived MSCs, AD-MSCs and MSCs derived from Wharton's jelly of the umbilical cord. They found comparable phenotype, proliferation, clonality and that γ-IFN induced IDO expression was greatest in UC-MSCs, supporting our findings as well. Kim, J H, et al, (Stem Cells International, 2018: 8429042), also showed superior immunosuppression and minimal HLA-DR expression in UC-MSCs compared to AD-MSCs and periodontal ligament-derived MSCs. Other reports of the comparison of MSC from various tissue sources also support biological advantages of UC-MSCs (Riordan, N H et al, J Transl Med 2018; 16: 57; Najar, M, et al, Cell Immunol. 2010; 264:171; Weiss, M L, et al, Stem Cells 2006; 24: 781; Arutyunyan, I, et al, Stem Cell International 2016; 2016: 6901286) Jin, H J, et al, (Int. J. Mol Sci 2013; 14: 17986), showed superior proliferation and anti-inflammatory properties of UC-MSCs compared to BM-MSCs and AD-MSCs.

Expanded MSCs showed measurable levels of cell-specific ATP content. However, cell-specific ATP expression was significantly higher in UC-MSCs supporting the assertion that they are the most potent type of MSC. Other studies have shown that ATP expression correlates with therapeutic outcomes in the transplantation of hematopoietic stem cells (Deskins D, et al, Stem Cells Transl Med. 2013; 2:151; Rich, IN Stem Cell Transl Med 2015; 4: 967).

Numerous clinical trials have been conducted and are presently ongoing for various MSC preparations (Carralho E, et al. Regen. Med. 2015; 10:1025; Freitag J, et al. BMC Musculoskeletal Disorders 2016; 17: 230; Neirinckx V, et al. Stem Cell Trans. Med 2013; 2: 284; Munir H and McGettrick, HM Stem Cells Dev. 2015; 24:2091). From the results reported here it would be expected that expanded UC-MSCs exhibit greater therapeutic benefit than other impure sources of MSCs such as bone marrow aspirate and stromal vascular fraction. Direct clinical comparisons from various sourced MSCs are lacking.

Mechanisms of stem cell therapy include paracrine effects from stem cell-derived biological factors eliciting anti-inflammatory & neural protective effects, differentiation of stem cells into other cellular lineages, and intercellular communication through tunneling nanotubes.

### Conclusion

Our results show bio-similarity between stem cells derived from adipose, bone marrow, placental and umbilical cord tissues regarding expansion, trilineage differentiation, and phenotypic characterization by flow cytometry according to the ISCT definition of MSCs. While all sources of MSCs also exhibited activity in potency assays including quantitative assessment of mitochondrial function and immunosuppression, cell migration and proliferation, there were clear differences. Our results revealed significant superiority of UC-derived MSCs as was also found in similar studies performed in several other laboratories. Age of the cells may be a factor in the overall performance of MSCs. Furthermore, the capacity to differentiate into neural stem cells varied between MSC derived from UC, adipose and placental tissues with UC derived MSCs expressing all NSC markers while adipose and placental-derived MSCs did not express GLAST under identical conditions. Thus, while MSCs from various tissues show similarity, there are also multiple characteristics of umbilical cord MSCs significantly superior to those derived from adipose, bone marrow or placental tissues. This suggests that UC-MSCs may also exhibit superior therapeutic benefit.

### Example 7: Genetically Modifying UC-MSC

A child is diagnosed with PTHS by a clinician who genotyped the child's TCF4 gene and performed an Observer-Reported Communication Ability Measure (ORCA) test. The next day, the child is administered a 6 mL pharmaceutical composition containing 1×10⁶ modified UC-MSCs, PRIME-XV® MSC FreeziS DMSO-Free cryopreservation medium, and a pharmaceutical diluent. The modified UC-MSCs are from a subject that does not have PTHS (e.g., the UC-MSCs are autologous) and have been genetically modified so that an additional functional copy of TCF4 is being expressed by a nucleic acid vector that was previously transfected into the UC-MSC. Administration of the pharmaceutical composition to the child occurs intravenously over the course of 20 minutes. Concurrently with the administration of the pharmaceutical composition, the child receives about 400 mg of the HDAC1 inhibitor, vorinostat, by tablet. The child continues to receive treatment with the pharmaceutical composition every three months, along with speech therapy. After four treatments over a 12 month period, an ORCA test is performed again on the child and indicates a 10% increase in the child's verbal communication skills, thereby indicating that the treatment is working.

On the fifth treatment, the child is administered a different pharmaceutical composition containing 2.5×10⁶ modified UC-MSCs, PRIME-XV® MSC FreeziS DMSO-Free cryopreservation medium, and a pharmaceutical diluent in a 1 mL volume. The modified UC-MSCs are from a subject that does not have PTHS (e.g., the UC-MSCs are autologous) and have been genetically modified so that two functional copies of TCF4 are being expressed by a circular RNA that was previously transfected into the UC-MSC. Additionally, the genetically modified UC-MSC has been cultured in 20 ng/mL of Wnt-3a-conditioned cell culture medium for two weeks. This pharmaceutical composition is administered to the child intravenously over the course of 2 minutes. The child continues to receive this treatment every six months.

### Example 8: Treating PTHS with UC-MSCs

An infant with PTHS can be treated according to the methods described herein. The infant may be diagnosed by a clinician, such as by measuring the level of TCF4 in the child's lymphocytes and discovering that the infant's TCF4 expression level is 50% lower than that of a parent, as determined by microarray analysis. Subsequent RNA sequencing of the infant's TCF4 gene may reveal the presence of a pathogenic mutation in TCF4. The infant may be administered (e.g., within a week or more of diagnosis) a 3 mL pharmaceutical composition containing 1.5×10⁶ UC-MSCs, MCDB-131 basal medium, and a pharmaceutical diluent. The UC-MSCs can be produced from a subject that does not have PTHS (e.g., the UC-MSCs are autologous) and that expresses a non-mutant TCF4 gene.

Prior to administration of the pharmaceutical composition, the infant may be treated with romidepsin, such as in an amount of about 14 mg/m², by intravenous infusion (e.g., over a 4-hour period). After the infusion, the infant's serum levels can be assessed to confirm a level of romidepsin of, e.g., about 500 μM. Administration of the pharmaceutical composition to the infant can be by intravenous infusion, such as over the course of 45 minutes. The infant may continue to receive treatment with the pharmaceutical composition as needed, such as once every three months, along with standard of care treatments. After 12 months, lymphocytes can again be isolated from the infant and tested for the level of TCF4, e.g., by microarray analysis. The results of the microarray analysis may show, e.g., a 10% increase in the infant's TCF4 express relative to the previous measurement, thereby indicating that the treatment is working.

### Example 9: Treating PTHS with Isolated Exosomes

An adult with PTHS can be treated (e.g., within one year of diagnosis) according to the methods described herein. The adult may be administered a 1 mL pharmaceutical composition containing 5×10⁹ isolated exosomes in a saline solution. The isolated exosomes may be about 80 to about 200 nm (e.g., 72 nm, 80 nm, 90 nm, 100 nm, 110 nm, 120 nm, 130 nm, 140 nm, 150 nm, 160 nm, 170 nm, 180 nm, 190 nm, 200 nm, 210 nm, or 220 nm) in size and express CD9, CD63, CD81, CD44, CD29, and CD142. Administration of the pharmaceutical composition to the adult may occur by IV drip over the course of about 60 minutes (e.g., 54 minutes to 66 minutes). The adult may continue to receive treatment with the pharmaceutical composition every three months (e.g., every 27, 28, 29, 30, 31, 32, or 33 days), along with standard of care treatment (e.g., speech therapy, cognitive therapy, and occupational therapy). After two treatments over a 6 month period (e.g., 54, 55, 56, 57, 58, 59, 60, 61, 62, or 63 days), the adult's lymphocytes are isolated and analyzed, e.g., by qPCR or microarray analysis, for TCF4 expression levels, which may increase by 5%, 10%, 15%, or 20% (or more) relative to the adult's TCF4 expression levels in lymphocytes isolated prior to receiving therapy.

On the fifth treatment, the adult with PTHS may be administered a 10 mL pharmaceutical composition containing 2.5×10⁶ modified UC-MSCs, cryopreservation medium, and a pharmaceutical diluent. The modified UC-MSCs may be from a subject that does not have PTHS (e.g., the UC-MSCs are autologous) and have been genetically modified (e.g., transfected with genetic material) so that a third functional copy of TCF4 is being expressed by e.g., a circular RNA that was previously transfected into the UC-MSC. Additionally, the genetically modified UC-MSC may be cultured in 20 ng/mL of Wnt-3a-conditioned cell culture medium for about two weeks (e.g., 13, 14, or 15 days) prior to administration. This pharmaceutical composition may be administered to the adult by intravenous infusion over the course of 50 minutes. The adult may continue to receive this treatment as needed, such as every six months for the life of the subject.

### Example 10: Treating PTHS with UC-MSC Secretome-Conditioned Cell Culture Medium

A subject (e.g., an infant, a child, or an adolescent) may be diagnosed with PTHS by a clinician who can genotype the subject's TCF4 gene and measure TCF4 expression levels by, e.g., RNA sequencing. Genotyping may reveal pathogenic mutation and RNA sequencing may reveal an expression level more than 20% (e.g., 20%, 25%, 30%, 35%, 40%, 45%, 50%, or more) lower than that of a control subject (e.g., a subject with a non-mutant TCF4 gene). The subject may then be administered (e.g., within one week of diagnosis) a 10 mL pharmaceutical composition containing UC-MSC secretome-conditioned cell culture medium. The composition may contain exosomes that express CD9, CD63, CD81, CD44, CD29, and CD142 and various proteins (e.g., GM-CSF, MIP-3a, IL-6, IL-8, MIP-1, and fractalkine) each at a concentration of about 100 μg/mL (e.g., 90 pg/mL to 110 pg/mL).

Administration of the pharmaceutical composition to the subject may occur intravenuously (e.g., over the course of 30 minutes). The subject may continue to receive treatment with the pharmaceutical composition as needed, such as once every three months. After eight treatments over a 24 month period, the subject's TCF4 expression levels can be measured by RNA sequencing and may show, e.g., a 100% increase in TCF4 expression, relative to the subject's TCF4 expression levels prior to receiving therapy. Treatment is then halted and standard of care treatment is applied.

### Example 11: Treating PTHS with Exosome-Depleted UC-MSC-Conditioned Cell Culture Medium

A subject may be diagnosed with PTHS by a clinician who genotyped the subject's TCF4 gene and measured TCF4 expression levels (e.g., TCF4 mRNA expression levels in lymphocytes by RNA sequencing). Genotyping may reveal a pathogenic mutation and RNA sequencing may reveal an expression level that is 75% lower than that of a control subject (e.g., a subject with a non-mutant TCF4 gene). An Observer-Reported Communication Ability Measure (ORCA) test may be used to corroborate the diagnosis. The subject may be administered a 2 mL pharmaceutical composition containing exosome-depleted UC-MSC-conditioned cell culture medium. The composition may not contain any exosomes between 140-200 nm in diameter; however the composition may contain GM-CSF, MIP-3a, IL-6, and IL-8 each at a concentration of about 400-500 μg/mL (e.g., 450 pg·mL) and MIP-1 and fractalkine at a concentration of 50-100 pg/mL (e.g., 75 μg/mL).

Administration of the pharmaceutical composition to the subject having PTHS may occur intravenuously (e.g., over the course of 35 minutes). The subject can continue to receive treatment with the pharmaceutical composition as needed, e.g., every three months. After eight treatments over a 24 month period, a second ORCA test may be employed to assess therapeutic efficacy. The second ORCA test may reveal an increase in the subject's cognitive abilities, as determined by a clinician, relative to the subject's first ORCA test. Treatment can be halted, and a standard of care treatment can then be applied.

### Other Embodiments

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations following, in general, the principles and including such departures from the invention that come within known or customary practice within the art to which the invention pertains and may be applied to the essential features hereinbefore set forth, and follows in the scope of the claims.

Other embodiments are within the claims.

1. A method of treating a subject with Pitt-Hopkins Syndrome (PTHS) comprising administering to the subject an effective amount of a composition comprising:
(a) a plurality of umbilical cord-derived human mesenchymal stem cells (UC-MSCs), wherein the UC-MSCs express transcription factor 4 (TCF4);
(b) a plurality of isolated exosomes about 80-200 nanometers (nm) in diameter, wherein the isolated exosomes are derived from the UC-MSCs;
(c) UC-MSC secretome-conditioned cell culture medium; and/or
(d) exosome-depleted UC-MSC-conditioned cell culture medium.

2. The method of claim 1, wherein the composition is administered intravenously, intra-articularly, intramuscularly, intranasally, or intrathecally.

3. The method of claim 2, wherein the composition is administered intravenously.

4. The method of claim 3, wherein the composition is administered intravenously by infusion.

5. The method of claim 1, wherein the composition is administered in a volume of about 0.5 milliliters (mL) to about 15 mL.

6. The method of claim 5, wherein the composition is administered in a volume of about 1 mL to about 10 mL.

7. The method of claim 6, wherein the composition is administered in a volume of about 1 mL, about 2 mL, about 3 mL, about 4 mL, about 5 mL, or about 6 mL.

8. The method of claim 1, wherein the composition is administered in a bolus or is administered over a period of about 1 minute to about 1 hour.

9. The method of claim 8, wherein the composition is administered over a period of about 1 minute to about 30 minutes.

10. The method of claim 9, wherein the composition is administered over a period of about 1 minute to about 10 minutes.

11. The method of claim 1, wherein the composition is administered at a frequency of once every one, two, three, four, five, or six months.

12. The method of claim 11, wherein the composition is administered at a frequency of once every three months.

13. The method of any one of claims 1-12, wherein the UC-MSCs are modified to increase expression of TCF4 mRNA and/or protein expression levels relative to unmodified UC-MSCs.

14. The method of claim 13, wherein the modified UC-MSCs comprise a nucleic acid vector, plasmid, circular RNA, or mRNA molecule comprising a nucleotide sequence encoding a TCF4 protein and/or a frizzled-signaling agonist.

15. The method of claim 14, wherein the frizzled-signaling agonist is Wnt-3a protein.

16. The method of any one of claims 1-15, wherein the method further comprises, prior to administering the composition, contacting the UC-MSCs with a Wnt pathway activator.

17. The method of claim 16, wherein the Wnt pathway activator is selected from the group consisting of a histone deacetylase 1 (HDAC1) inhibitor, a Wnt-signaling agonist, a frizzled-signaling agonist, and a glycogen synthase kinase-3p (GSK-3B3) inhibitor.

18. The method of claim 17, wherein:
(a) the HDAC1 inhibitor is selected from the group consisting of vorinostat, romidepsin, belinostat, panobinostat, valproic acid, entinostat, curcumin, curcumin, quercetin, and RG2833;
(b) the Wnt-signaling agonist is L-Quebrachital;
(c) the frizzled-signaling agonist is a Wnt agonist-1 protein or a Wnt-3a protein; and/or
(d) the GSK-3B3 inhibitor is selected from the groups consisting of indirubin-3′-oxime, laduviglusib (CHIR-99821), and KY19382.

19. The method of any one of claims 16-18, wherein the contacting is for about 1 day to about 6 weeks.

20. The method of claim 19, wherein the contacting is for about 1-3 weeks.

21. The method of claim 20, wherein the contacting is for about 2 weeks.

22. The method of claim 18, wherein the HDAC1 inhibitor is at a concentration of about 1 nanomolar (nM) to about 10 micromolar (μM).

23. The method of claim 22, wherein the HDAC1 inhibitor is at a concentration of about 100 nM to about 1 μM.

24. The method of claim 23, wherein the HDAC1 inhibitor is at a concentration of about 400-600 nm, such as about 500 nM.

25. The method of claim 18, wherein the Wnt-3a protein is at a concentration of about 5 ng/mL to about 20 ng/mL.

26. The method of any one of claims 1-25, wherein, prior to administration of the composition, the subject is administered a Wnt pathway activator.

27. The method of claim 26, wherein the Wnt pathway activator is selected from the group consisting of a HDAC1 inhibitor, a Wnt-signaling agonist, a frizzled-signaling agonist, and a GSK-3B3 inhibitor.

28. The method of claim 27, wherein:
(a) the HDAC1 inhibitor is selected from the group consisting of vorinostat, romidepsin, belinostat, panobinostat, valproic acid, entinostat, curcumin, curcumin, quercetin, and RG2833;
(b) the Wnt-signaling agonist is L-Quebrachital;
(c) the frizzled-signaling agonist is a Wnt agonist-1 protein or a Wnt-3a protein; and/or
(d) the GSK-3B3 inhibitor is selected from the groups consisting of indirubin-3′-oxime, laduviglusib (CHIR-99821), and KY19382.

29. The method of claim 28, wherein the HDAC1 inhibitor is administered to the subject in an amount sufficient to achieve a serum concentration of the HDAC1 inhibitor of about 0.1 nM to about 1,000 nM, such as about 500 nM.

30. The method of claim 29, wherein the Wnt-3a protein is administered to the subject in an amount sufficient to achieve a serum concentration of the Wnt-3a protein of about 5 ng/mL to about 20 ng/mL.

31. The method of any one of claims 1-30, wherein the method further comprises administering to the subject a Wnt pathway activator concurrently with or following administration of the composition.

32. The method of claim 31, wherein the Wnt pathway activator is selected from the group consisting of a HDAC1 inhibitor, a Wnt-signaling agonist, a frizzled-signaling agonist, and a GSK-3B3 inhibitor.

33. The method of claim 32, wherein:
(a) the HDAC1 inhibitor is selected from the group consisting of vorinostat, romidepsin, belinostat, panobinostat, valproic acid, entinostat, curcumin, curcumin, quercetin, and RG2833;
(b) the Wnt-signaling agonist is L-Quebrachital;
(c) the frizzled-signaling agonist is a Wnt agonist-1 protein or a Wnt-3a protein; and/or
(d) the GSK-3B13P inhibitor is selected from the groups consisting of indirubin-3′-oxime, laduviglusib (CHIR-99821), and KY19382.

34. The method of claim 33, wherein the subject is administered:
(a) vorinostat in an amount of about 400 mg and/or in an amount sufficient to achieve geometric mean values of a maximum plasma concentration (Cₘₐₓ) and an area under the plasma concentration versus time curve (AUC₀₋ᵢₙₜ) of about 1.2±0.53 μM and about 6.0±2.0 μM*hr, respectively;
(b) romidepsin in an amount of about 14 mg/m² IV over a 4-hour period, such as on days 1, 8, and 15 of a 28-day cycle and/or in an amount sufficient to achieve geometric mean values of a maximum plasma concentration (Cₘₐₓ) and an area under the plasma concentration versus time curve (AUC₀₋ᵢₙₜ of about 377 ng/mL and about 1549 ng*hr/mL, respectively;
(c) belinostat in an amount of about 1,000 mg/m² over a 30 minute period, such as on days 1-5 of a 21-day cycle;
(d) panobinostat in an amount of about 20 mg every other day, such as on days 1, 3, 5, 8, 10, and 12 of a 21-day cycle;
(e) valproic acid in an amount of about 10 to 60 mg/kg/day;
(f) entinostat in an amount of about 2 mg/m² to about 12 mg/m² per day;
(g) curcumin in an amount of about 1 g to about 8 g per day;
(h) quercetin in an amount of about 250 mg to about 1000 mg per day; and/or
(i) RG2833 in an amount of about 30 mg to about 240 mg per day.

35. The method of any one of claims 1-34, wherein the subject is an infant, child, or adolescent.

36. The method of claim 35, wherein:
(a) the infant is less than one year of age;
(b) the child is between one year and 10 years of age; or
(c) the adolescent is over 10 years and under 19 years of age.

37. The method of claim 36, wherein:
(a) the infant is administered the composition by intravenous infusion at about 1×10⁶ to 2.5×10⁶ UC-MSCs per kilogram (kg) of body about 3 to 4 times per year depending on need;
(b) the child is administered the composition by intravenous infusion at about 1×10⁶ to 2.5×10⁶ UC-MSCs per kilogram (kg) of body about 3 to 4 times per year depending on need; or
(c) the adolescent is administered the composition by intravenous infusion at about 1×10⁶ to 2.5×10⁶ UC-MSCs per kilogram (kg) of body about 3 to 4 times per year depending on need.

38. The method of any one of claims 1-37, wherein the subject exhibits an impairment in motor function, communication, sleep, gastrointestinal health, breathing, cognition, and/or adaptive behavior.

39. The method of claim 38, wherein:
(a) the motor function impairment is determined by one or more of a Vineland Motor Subscale-3 caregiver interview, a Bayley Scales of Infant Development (BSID-4) questionnaire, a video capture of gait in coronal and sagittal plane, a Functional Independence Measure for Children (WeeFIM), and an Observer-Reported Communication Ability Measure (ORCA);
(b) the communication impairment is determined by WeeFIM and/or ORCA;
(c) the sleep impairment is determined by a sleep diary questionnaire;
(d) the gastrointestinal impairment is determined by a gastrointestinal health questionnaire;
(e) the breathing impairment is determined by spirometry;
(f) the cognition impairment is determined by a BSID-4 questionnaire; and/or
(g) the adaptive behavior impairment is determined by one or more of a Q-global Vineland assessment, a Vineland behavioral scalers questionnaire, or an Aberrant Behavior Checklist-2.

40. The method of any one of claims 1-39, wherein the subject has:
(a) a reduced TCF4 expression level in excitatory neurons, inhibitory neurons, astrocytes, oligodendrocytes and/or lymphocytes relative to a healthy subject; and/or
(b) a monoallelic mutation or deletion in TCF4 that reduces TCF4 expression, relative to a healthy subject without the mutation or deletion in TCF4.

41. The method of any one of claims 1-40, wherein the composition increases TCF4 expression in the brain of the subject relative to the TCF4 expression level prior to administering of the composition.

42. The method of any one of claims 1-41, wherein the composition comprises:
(a) about 5×10⁵ to 5×10⁶ said UC-MSCs;
(b) a concentration of about 5×10⁹ to 5×10¹⁰ said exosomes per mL, said exosomes expressing cluster of differentiation (CD)-9, CD63, CD81, CD29, CD44 and/or CD144;
(c) about 100 μg/mL to about 5000 μg/mL of granulocyte-macrophage colony-stimulating factor (GM-CSF), macrophage inflammatory protein (MIP)-3 alpha (MIP-3a), IL-6, and IL-8 and about 10 μg/mL to about 1000 μg/mL of fractalkine and MIP-1; and/or
(d) a pharmaceutically acceptable carrier, excipient, or diluent,
wherein, optionally, the pharmaceutical composition does not contain DMSO.

43. The method of claim 42, wherein the composition further comprises:
(a) a cryopreservation medium;
(b) a basal medium; and/or
(c) a saline solution.

44. The method of claim 43, wherein:
(a) the cryopreservation medium is PRIME-XV® MSC FreeziS DMSO-Free medium; and/or
(b) the basal medium is MCDB-131.

45. The method of any one of claims 42-44, wherein the pharmaceutical composition comprises about 1×10⁶ to 2.5×10⁶ said UC-MSCs.

46. The method of claim 45, wherein the pharmaceutical composition comprises about 1×10⁶ said UC-MSCs.

47. The method of any one of claims 42-46, wherein the pharmaceutical composition comprises a concentration of about 1×10¹⁰ to 5×10¹⁰ said isolated exosomes per mL.

48. The method of any one of claims 1-47, wherein:
(a) the exosomes express CD9, CD63, and CD81;
(b) the exosomes express CD44, CD29, and CD412; and/or
(c) the exosomes do not express CD45, CD11 b, CD14, CD19, CD34, CD79a, CD126, and human leukocyte antigen-DR isotype (HLD-DR).

49. The method of claim 42, wherein the pharmaceutical composition comprises about 100 pg/mL to about 1000 pg/mL of GM-CSF, MIP-3a, IL-6, and IL-8 and about 10 pg/mL to about 100 pg/mL of fractalkine and MIP-1.

50. The method of claim 49, wherein the pharmaceutical composition comprises about 1000 μg/mL of GM-CSF, MIP-3a, IL-6, and IL-8 and about 100 μg/mL of fractalkine and MIP-1.

51. A pharmaceutical composition comprising:
(a) a plurality of UC-MSCs, wherein the UC-MSCs express TCF4;
(b) a plurality of isolated exosomes of about 80-200 nm in diameter, wherein the isolated exosomes are derived from the UC-MSCs and express CD9, CD63, CD81, CD44, CD29 and CD142;
(c) UC-MSC secretome-conditioned cell culture medium; and/or
(d) exosome-depleted UC-MSC-conditioned cell culture medium,
wherein the composition further comprises one or more Wnt pathway activators.

52. The method of claim 51, wherein the Wnt pathway activator is selected from the group consisting of a HDAC1 inhibitor, a Wnt-signaling agonist, a frizzled-signaling agonist, and a GSK-3B3 inhibitor.

53. The method of claim 52, wherein:
(a) the HDAC1 inhibitor is selected from the group consisting of vorinostat, romidepsin, belinostat, panobinostat, valproic acid, entinostat, curcumin, curcumin, quercetin, and RG2833;
(b) the Wnt-signaling agonist is L-Quebrachita(c) the frizzled-signaling agonist is a Wnt agonist-1 protein or a Wnt-3a protein; and/or
(d) the GSK-3B3 inhibitor is selected from the groups consisting of indirubin-3′-oxime, laduviglusib (CHIR-99821), and KY19382.

54. The pharmaceutical composition of any one of claims 51-53, wherein the UC-MSCs are modified to increase expression of TCF4 mRNA and/or protein expression levels relative to unmodified UC-MSCs.

55. The pharmaceutical composition of claim 54, wherein the modified UC-MSCs comprise a nucleic acid vector, plasmid, circular RNA, or mRNA molecule comprising a nucleotide sequence encoding a TCF4 protein and/or a frizzled-signaling agonist.

56. The pharmaceutical composition of any one of claims 51-55 further comprising:
(a) about 5×10⁵ to 5×10⁶ of said UC-MSCs;
(b) a concentration of about 5×10⁹ to 5×10¹⁰ said exosomes per mL;
(c) about 100 pg/mL to about 5000 pg/mL of GM-CSF, MIP-3a, IL-6, and IL-8 and about 10 pg/mL to about 1000 pg/mL of fractalkine and MIP-1; and/or
(d) a pharmaceutically acceptable carrier, excipient, or diluent,
wherein, optionally, the pharmaceutical composition does not contain DMSO.

57. The pharmaceutical composition of claim 56 further comprising:
(a) a cryopreservation medium;
(b) a basal medium; and/or
(c) a saline solution.

58. The pharmaceutical composition of claim 57, wherein:
(a) the cryopreservation medium is PRIME-XV® MSC FreeziS DMSO-Free medium; and/or
(b) the basal medium is MCDB-131.

59. The pharmaceutical composition of any one of claims 56-58, wherein the pharmaceutical composition comprises about 1×10⁶ to 2.5×10⁶ of said UC-MSCs.

60. The pharmaceutical composition of claim 59, wherein the pharmaceutical composition comprises about 1×10⁶ of said UC-MSCs.

61. The pharmaceutical composition of any one of claims 56-60, wherein the pharmaceutical composition comprises a concentration of about 1×10¹⁰ to 5×10¹⁰ of said isolated exosomes per mL.

62. The pharmaceutical composition of claim 52, wherein the HDAC1 inhibitor is selected from the group consisting of vorinostat, romidepsin, belinostat, panobinostat, valproic acid, entinostat, curcumin, quercetin, and RG2833.

63. The pharmaceutical composition of claim 62, wherein the composition comprises:
(a) vorinostat in an amount of about 400 mg;
(b) romidepsin in an amount providing about 14 mg/m² of a subject's body surface area;
(c) belinostat in an amount providing about 1,000 mg/m² of the subject's body surface area;
(d) panobinostat in an amount of about 20 mg;
(e) valproic acid in an amount providing about 10 to 60 mg/kg of the subject's body weight;
(f) entinostat in an amount providing about 2 mg/m² to about 12 mg/m² of the subject's body surface area;
(g) curcumin in an amount of about 1 g to about 8 g;
(h) quercetin in an amount of about 250 mg to about 1000 mg; and/or
(i) RG2833 in an amount of about 30 mg to about 240 mg.

64. The pharmaceutical composition of any one of claims 51-63, wherein the composition is formulated in a volume of about 0.5 mL to about 15 mL.

65. The pharmaceutical composition of claim 64, wherein the composition is formulated in a volume of about 1 mL to about 10 mL.

66. The pharmaceutical composition of claim 65, wherein the composition is formulated in a volume of about 1 mL, about 2 mL, about 3 mL, about 4 mL, about 5 mL, or about 6 mL.

67. The pharmaceutical composition of any one of claims 51-66, wherein:
(a) the exosomes express CD9, CD63, and CD81;
(b) the exosomes express CD44, CD29, and CD142; and/or
(c) the exosomes do not express CD45, CD11 b, CD14, CD19, CD34, CD79a, CD126, and HLD-DR.

68. The pharmaceutical composition of any one of claims 56-67, wherein the pharmaceutical composition comprises about 100-1000 pg/mL of GM-CSF, MIP-3a, IL-6, and IL-8 and about 10-100 pg/mL of fractalkine and MIP-1.

69. The pharmaceutical composition of claim 68, wherein the pharmaceutical composition comprises about 1000 μg/mL of GM-CSF, MIP-3a, IL-6, and IL-8 and about 100 μg/mL of fractalkine and MIP-1.

70. A pharmaceutical composition for use in treating PTHS comprising:
(a) a plurality of UC-MSCs, wherein the UC-MSCs express TCF4;
(b) a plurality of isolated exosomes of about 80-200 nm in diameter, wherein the isolated exosomes are derived from the UC-MSCs and express CD9, CD63, CD81, CD44, CD29 and CD142;
(c) UC-MSC secretome-conditioned cell culture medium; and/or
(d) exosome-depleted UC-MSC-conditioned cell culture medium,
wherein the composition further comprises one or more Wnt pathway activators.

71. The pharmaceutical composition for use of claim 70, wherein the Wnt pathway activator is selected from the group consisting of a HDAC1 inhibitor, a Wnt-signaling agonist, a frizzled-signaling agonist, and a GSK-3B3 inhibitor.

72. The pharmaceutical composition for use of claim 71, wherein:
(a) the HDAC1 inhibitor is selected from the group consisting of vorinostat, romidepsin, belinostat, panobinostat, valproic acid, entinostat, curcumin, curcumin, quercetin, and RG2833;
(b) the Wnt-signaling agonist is L-Quebrachital;
(c) the frizzled-signaling agonist is a Wnt agonist-1 protein or a Wnt-3a protein; and/or
(d) the GSK-3B3 inhibitor is selected from the groups consisting of indirubin-3′-oxime, laduviglusib (CHIR-99821), and KY19382.

73. The pharmaceutical composition for use of any one of claims 70-72, wherein the UC-MSCs are modified to increase expression of TCF4 mRNA and/or protein expression levels relative to unmodified UC-MSCs.

74. The pharmaceutical composition for use of claim 73, wherein the modified UC-MSCs comprise a nucleic acid vector, plasmid, circular RNA, or mRNA molecule comprising a nucleotide sequence encoding a TCF4 protein and/or a frizzled-signaling agonist.

75. The pharmaceutical composition for use of any one of claims 70-74 further comprising:
(a) about 5×10⁵ to 5×10⁶ of said UC-MSCs;
(b) a concentration of about 5×10⁹ to 5×10¹⁰ said exosomes per mL;
(c) about 100-5000 pg/mL of GM-CSF, MIP-3a, IL-6, and IL-8 and about 10-1000 pg/mL of fractalkine and MIP-1; and/or
(d) a pharmaceutically acceptable carrier, excipient, or diluent,
wherein, optionally, the pharmaceutical composition does not contain DMSO.

76. The pharmaceutical composition for use of claim 75 further comprising:
(a) a cryopreservation medium;
(b) a basal medium; and/or
(c) a saline solution.

77. The pharmaceutical composition for use of claim 76, wherein:
(a) the cryopreservation medium is PRIME-XV® MSC FreeziS DMSO-Free medium; and/or
(b) the basal medium is MCDB-131.

78. The pharmaceutical composition for use of any one of claims 70-75, wherein the pharmaceutical composition comprises about 1×10⁶ to 2.5×10⁶ of said UC-MSCs.

79. The pharmaceutical composition for use of claim 78, wherein the pharmaceutical composition comprises about 1×10⁶ of said UC-MSCs.

80. The pharmaceutical composition for use of any one of claims 70-79, wherein the pharmaceutical composition comprises a concentration of about 1×10¹⁰ to 5×10¹⁰ of said isolated exosomes per mL.

81. The pharmaceutical composition for use of claim 72, wherein the HDAC1 inhibitor is selected from the group consisting of vorinostat, romidepsin, belinostat, panobinostat, valproic acid, entinostat, curcumin, quercetin, and RG2833.

82. The pharmaceutical composition for use of claim 81, wherein:
(a) vorinostat in an amount of about 400 mg;
(b) romidepsin in an amount of about 14 mg/m² of a subject's body surface area;
(c) belinostat in an amount of about 1,000 mg/m² of the subject's body surface area;
(d) panobinostat in an amount of about 20 mg;
(e) valproic acid in an amount of about 10 to 60 mg/kg of the subject's body weight;
(f) entinostat in an amount of about 2 mg/m² to about 12 mg/m² of the subject's body surface area;
(g) curcumin in an amount of about 1 g to about 8 g;
(h) quercetin in an amount of about 250 mg to about 1000 mg; and/or
(i) RG2833 in an amount of about 30 mg to about 240 mg.

83. The pharmaceutical composition for use of any one of claims 70-82, wherein the composition is formulated in a volume of about 0.5 mL to about 15 mL.

84. The pharmaceutical composition for use of claim 83, wherein the composition is formulated in a volume of about 1 mL to about 10 mL.

85. The pharmaceutical composition for use of claim 84, wherein the composition is formulated in a volume of about 1 mL, about 2 mL, about 3 mL, about 4 mL, about 5 mL, or about 6 mL.

86. The pharmaceutical composition for use of any one of claims 70-85, wherein:
(a) the exosomes express CD9, CD63, and CD81;
(b) the exosomes express CD44, CD29, and CD142; and/or
(c) the exosomes do not express CD45, CD11 b, CD14, CD19, CD34, CD79a, CD126, and HLD-DR.

87. The pharmaceutical composition for use of any one of claims 75-86, wherein the pharmaceutical composition comprises about 100-1000 pg/mL of GM-CSF, MIP-3a, IL-6, and IL-8 and about 10-100 pg/mL of fractalkine and MIP-1.

88. The pharmaceutical composition for use of claim 87, wherein the pharmaceutical composition comprises about 1000 pg/mL of GM-CSF, MIP-3a, IL-6, and IL-8 and about 100 pg/mL of fractalkine and MIP-1.
